(19) United States
(12) Patent Application Publication
(10) Pub. No.: US 2018/0320153 A9
(43) Pub. Date: Nov. 8, 2018

(54) DIRECTED DIFFERENTIATION OF PLURIPOTENT STEM CELLS BY BACTERIAL INJECTION OF TALEN PROTEINS

(71) Applicant: University of Florida Research Foundation, Incorporated, Gainesville, FL (US)

(72) Inventors: Shouguang Jin, Gainesvelle, FL (US); Naohiro Terada, Gainesville, FL (US); Fang Bai, Gainesville, FL (US)

(73) Assignee: University of Florida Research Foundation, Incorporated, Gainesville, FL (US)

(21) Appl. No.: 15/566,460

(22) PCT Filed: Apr. 15, 2016
(86) PCT No.: PCT/US16/27904

§ 371 Date: Oct. 13, 2017

Related U.S. Application Data
(60) Provisional application No. 62/148,154, filed on Apr. 15, 2015.
(60) Provisional application No. 62/188,339, filed on Jul. 2, 2015.

(51) Int. Cl. C12N 9/22; C07K 14/21; C12N 15/78; C12N 5/077

(52) U.S. Cl. CPC C12N 9/22; C07K 14/21; C12N 15/78; C12N 5/0657; C12N 2501/16; C12N 2501/60; C12N 2506/02; C12N 2510/00; C07K 2319/80

(57) ABSTRACT

In some aspects, the disclosure relates to methods and compositions for delivery of proteins into mammalian cells. In some embodiments, the disclosure provides a genetically engineered bacterium that may be useful for delivery of proteins into mammalian cells. In some aspects, the disclosure relates to improved methods of bacterially-mediated protein delivery.

### RELATED APPLICATIONS

This application is a National Stage Application of PCT/US2016/027904, filed Apr. 15, 2016, entitled “DIRECTED DIFFERENTIATION OF PLURIPOTENT STEM CELLS BY BACTERIAL INJECTION OF TALEN PROTEINS”, which claims the benefit under 35 U.S.C. § 119(e) of U.S. Provisional Application Ser. No. 62/188,339, filed Jul. 2, 2015, entitled “DIRECTED DIFFERENTIATION OF PLURIPOTENT STEM CELLS BY BACTERIAL INJECTION OF DEFINED TRANSCRIPTION FACTORS”, and Provisional Application Ser. No. 62/148,154, filed Apr. 15, 2015, entitled “GENE EDITING IN PLURIPOTENT STEM CELLS BY BACTERIAL INJECTION OF TALEN PROTEINS”, the entire content of each application which is incorporated by reference herein.

### FEDERALLY SPONSORED RESEARCH

The invention was made with government support under Grant No. GM091238 awarded by the National Institutes of Health. The government has certain rights in the invention.

### BACKGROUND OF INVENTION

Many currently used methods for delivering proteins into cells require the delivery of nucleic acid-based (e.g., plasmid) or viral vectors. Drawbacks of using such vectors include inefficient delivery, especially of multiple proteins, lack of control for protein half-life, risk of undesirable integration of delivery vector into the host genome, and delivery-associated cytotoxicity. These challenges limit the usefulness of current methods, particularly in the context of sensitive cell types, such as stem cells. Thus, new compositions and methods for protein delivery into mammalian cells are needed.

### SUMMARY OF INVENTION

The disclosure relates, in part, to compositions and methods for improved delivery of proteins into mammalian cells. The disclosure is based, in part, on the recognition that genetically modified bacteria are capable of delivering proteins to sensitive mammalian cell types. Aspects of the disclosure are useful to deliver proteins (e.g., genome editing proteins) to cells (e.g., stem cells), for example to direct differentiation of stem cells.

In some aspects, the disclosure provides a Pseudomonas bacterium that is modified to deliver one or more recombinant proteins to heterologous cells (e.g., mammalian cells). In some embodiments, the modified Pseudomonas bacterium includes a polynucleotide encoding a fusion protein, wherein the fusion protein includes a heterologous protein fused to a bacterial secretion domain (e g., a Pseudomonas secretion domain) In some embodiments, the modified Pseudomonas bacterium is deficient for exoS, exoT, exoY, and popN (e.g., a ASTYN Pseudomonas bacterium). In some embodiments, the modified Pseudomonas bacterium is deficient for exoS, exoT, exoY, and popN and also is deficient for one or more of xcpQ, lasR-I, rhlR-I, and ndk.

In some embodiments, the Pseudomonas bacterium is a ASTYN Pseudomonas bacterium (e.g., a Pseudomonas bacterium in which the genome has been modified to remove naturally occurring T3SS genes, such as S, T, Y, and N) that also is deficient in at least one gene selected from the group consisting of xcpQ, lasR-I, rhlR-I, and ndk. In some embodiments, the bacterium lacks one or more (e.g., all) functional xcpQ, lasR-I, rhlR-I, and ndk proteins.

A modified Pseudomonas bacterium described by the disclosure is useful for the delivery of one or more transcription factors to a cell or cells. The cell or cells can be in vitro or in vivo. In some embodiments, a modified Pseudomonas bacterium delivers one or more transcription factors that induce differentiation of cells (e.g., differentiation of stem cells or fibroblasts into cardiomyocytes). For example, in some embodiments, the modified Pseudomonas bacterium delivers a transcription factor selected from the group consisting of Gata4 (SEQ ID NO: 20), Mef2c (SEQ ID NO: 21), and Tbx5 (SEQ ID NO:22). In some embodiments, each transcription factor (e.g., Gata4, Mef2c, and Tbx5) is fused to a bacterial secretion domain (e.g., a Pseudomonas secretion domain), such as ExoS54. In some embodiments, the transcription factor delivered by the modified Pseudomonas bacterium is ExoS54-Gata4 (SEQ ID NO: 23), ExoS54-Mef2c (SEQ ID NO: 24), or ExoS54-Tbx5 (SEQ ID NO: 25).

A modified Pseudomonas bacterium described by the disclosure is also useful for the delivery of one or more genome editing proteins. In some embodiments, gene editing proteins are fused to a bacterial secretion domain (e.g., a Pseudomonas secretion domain), such as ExoS54. In some embodiments, the genome editing protein is larger than 100 kDa in size. In some embodiments, the genome editing protein is a Transcription activator-like effector nuclease (TALEN) or a CRISPR/Cas protein.

In some embodiments, the polynucleotide encoding a fusion protein is on a plasmid or other nucleic acid vector. In some embodiments, the polynucleotide encoding a fusion protein is integrated into the genome of the bacterium.

In some embodiments, the Pseudomonas is P. aeruginosa, P. alcaligenes, P. anguilliseptica, P. citronellolis, P. flavescens, P. jinjuensis, P. mendocina, P. nitroreducens, P. oleovorans, P. pseudoalcaligenes, P. resinovorans, or P. straminae. In some embodiments, the Pseudomonas is P. aeruginosa. In some embodiments, the P. aeruginosa is PAK-J.

In some embodiments, the bacterial secretion domain is ExoS17, ExoS54, ExoS96, or ExoS234. In some embodiments, the bacterial secretion domain is ExoS54. In some embodiments, the bacterial secretion domain is represented by SEQ ID NO: 6.

In some embodiments, the Pseudomonas bacteria are used to deliver proteins to a recipient cell. The cell can be in vitro or in vivo. A recipient cell can be a mammalian cell. In some embodiments, a recipient cell is a human cell, for example a human stem cell. In some embodiments, the cell is a fibroblast, for example a human fibroblast.

In some embodiments, the bacterium exhibits reduced cytotoxicity to human stem cells compared to bacteria that are not deficient for xcpQ, lasR-I, rhlR-I, and/or ndk proteins (e.g., in the context of a ASTYN background). In some embodiments, the human stem cells are embryonic stem cells (hESCs) and/or induced pluripotent stem cells (hiPSCs).

Methods of delivering proteins to cells are also described herein. Accordingly, in some aspects, the disclosure provides a method of delivering one or more proteins into one or more isolated cells, by incubating the cell or cells with a Pseudomonas bacterium deficient in exoS, exoT, exoY and popN genes, wherein the bacterium is also deficient for one or more of the following genes: xcpQ, lasR-I, rhlR-I, and ndk, the bacterium comprising a polynucleotide encoding a fusion protein, wherein the fusion protein comprises a heterologous protein fused to a bacterial secretion domain; and incubating the isolated cell or cells for a period of time sufficient to deliver the one or more proteins into the cell or cells.

In some embodiments, the method further comprises transfecting the one or more isolated cells with a rescue construct. In some embodiments, a rescue construct can be used to provide a replacement gene for a gene targeted by one or more genome editing proteins. A rescue construct can be single-stranded polynucleotide or a double-stranded polynucleotide. In some embodiments, a rescue construct is a single-stranded oligonucleotide DNA (ssODN). The one or more isolated cells can be transfected with a rescue construct before, after, or simultaneously, to contact with the bacterium. In some embodiments, the rescue construct is delivered separately from the bacterium. In some embodiments, the rescue construct is expressed by the bacterium.

In some aspects, the disclosure relates to a method for inducing differentiation of a cell or cells to a cardiomyocyte, the method comprising incubating the cell or cells with a first modified Pseudomonas bacterium; incubating the cell or cells with a second modified Pseudomonas bacterium; and, incubating the cell or cells with a third modified Pseudomonas bacterium, wherein the first bacterium expresses Gata4 or a Gata4 fusion protein (e.g., ExoS54-Gata4), the second bacterium expresses Mef2c or a Mef2C fusion protein (e.g., ExoS54-Mef2c), and the third bacterium expresses Tbx5 or a Tbx5 fusion protein (e.g., ExoS54-Tbx5). In some embodiments of the method, the cell or cells are selected from the group consisting of stem cell(s) and fibroblast(s).

In some embodiments, the method further comprises washing the cell or the cells to remove the bacteria. In some embodiments, the method further comprises incubating the cell or cells with the first bacterium, the second bacterium, and the third bacterium, a second time. Wash and incubation steps can be repeated three, four, five, six or more times.

In some embodiments, the method further comprises incubating the cell or cells with a growth factor. In some embodiments, the growth factor is a growth factor associated with differentiation of stem cells (e.g., differentiation of stem cells into cardiomyocytes). In some embodiments, the growth factor is Nodal. In some embodiments, the growth factor is Activin A.

In some embodiments, the absolute multiplicity of infection (MOI) of bacteria to a target cell ranges from about 10 to about 1000. In some embodiments, the relative MOI of bacteria delivering different proteins ranges from about 1:1 to about 1:100. In some embodiments, the relative multiplicity of infection (MOI) ratio of the first bacterium to target cells: MOI of the second bacterium to target cells: MOI of the third bacterium to target cells ranges from 1:1:1 to 4:1:2.5.

In some embodiments, Gata4 protein or a Gata4 fusion protein (e.g., ExoS54-Gata4, SEQ ID NO: 23), Mef2c protein or a Mef2C fusion protein (e.g., ExoS54-Mef2c, SEQ ID NO: 24), and/or Tbx5 protein or a Tbx5 fusion protein (e.g., ExoS54-Tbx5, SEQ ID NO: 25) delivered to the cell or cells has an intracellular half-life of between about 4 and about 6 hours.

In some embodiments, incubating the cell or cells with at least one modified Pseudomonas bacterium results in expression of sarcomeric α-actinin, cardiac actin and/or troponin (e.g., troponin T) by the cell or cells.

In some aspects, the disclosure relates to a cardiomyocyte or cardiomyocytes produced by a method as described by the disclosure.

### BRIEF DESCRIPTION OF DRAWINGS

FIGS. 1A-1C show bacterial T3SS-mediated injection of TALEN proteins into mouse embryonic stem cells (mES). FIG. 1A shows a scheme of TALEN binding sites on a gfp gene. The left and right TALEN binding sequences are separated by a spacer sequence. The sequences, from top to bottom, correspond to SEQ ID NOs: 46-47. FIG. 1B shows a Western blot by anti-FLAG antibody of nuclear proteins from EB5 cells that were infected with the indicated bacterial strains at an MOI of 100 for 3 hours. For ASTY/TALEN1&2, the total MOI was 200. FIG. 1C shows Western blot by anti-FLAG antibody of nuclear proteins extracted from EB5 cells that were infected with PAK-JΔSTY/pExoS54-FLAG-TALEN1 and PAK-JΔSTY/pExoS54-FLAG-TALEN2 at an overall MOI of 200 for 3 hours.

FIGS. 2A-2C show data related to functional analysis of the bacterially injected TALENs. FIG. 2A shows fluorescence intensities of control EB5 cells (EB5), EB5 cells transfected with eukaryotic expression plasmids encoding the TALENs, EB5 cells infected by PAK-JΔSTY/pExoS54-FLAG-TALEN1 (TALEN1) or both PAK-JΔSTY/pExoS54-FLAG-TALEN1 and PAK-JΔSTY/pExoS54-FLAG-TALEN2 (TALEN1&2). Cells were analyzed by flow cytometry three days after the transfection or injection. FIG. 2B shows a representative GFP-negative EB5 cell colony (arrow) 3 days after bacterial delivery of the gfp-targeting TALEN protein pair, observed under fluorescence microscope. FIG. 2C shows a sequence alignment of the TALEN-targeting region among the GFP-negative EB5 cells following bacterial delivery of the gfp-targeting TALEN protein pair. The sequences, from top to bottom, correspond to SEQ ID NOs: 48-53.

FIGS. 3A-3C show data related to factors influencing the bacterial delivery of TALEN proteins. FIG. 3A shows the number of EB5 cells surviving infections of PAK-JΔSTY/pExoS54-FLAG-TALEN1 and PAK-JΔSTY/pExoS54-FLAG-TALEN2 (1:1 ratio) at the indicated MOI for 3 hours. The number of surviving cells was compared to no infection control by two-sample t-test. *P.<0.05; **P.<0.001; ***P<0.0001. Error bars represent standard deviations of triplicate assays. FIG. 3B shows data related to TALEN proteins injected into the EB5 cells after infection at the indicated MOI for 3 hours. Nuclear protein extracts from the same number surviving cells were prepared and subjected to Western blot analysis by anti-FLAG antibody. FIG. 3C shows FACS analysis results of EB5 cells three days post the TALEN injection at indicated MOI for 3 hours. The FACS data of infected cell populations were compared to no injection control by two-sample t-test. *P<0.05; **P.<0.001; ***P<0.0001. Error bars represent standard deviations of triplicate assays.

FIGS. 4A-4F show TALEN-mediated single-base change of gfp gene on genomic DNA. FIG. 4A shows strategy of single-base modification in gfp gene. The 72 base long single-stranded oligonucleotide DNA (ssODNs) template with a single base change from the wild type sequence, introduces a stop codon as well as a BfaI restriction enzyme digestion site, while second ssODN removes the stop codon and adds a new Sad restriction enzyme digestion site. The sequences, from top to bottom, correspond to SEQ ID NOs: 54, 17, 55, 18, and 56. FIG. 4B shows FACS analysis of fluorescence cell population three days after either transfection of ssODN-1 and eukaryotic expression plasmids encoding the TALEN pair or transfection of ssODN-1 followed by injection of TALEN proteins by P. aeruginosa. As a control, untreated EB5 cell is shown (EB5). Percentages of the GFP-negative cells in the whole population are shown. FIG. 4C shows a 350 bp fragment encompassing the TALEN-targeting region was amplified by PCR from GFP-negative EB5 cells that were FACS-Sorted after either transfection of ssODN-1 and TALEN coding plasmids or ssODN-1 transfection followed by TALEN protein injection. The PCR products were subjected to 2% agarose electrophoresis with (+) or without (−) digestion by BfaI restriction enzyme. Uninfected EB5 cell was used as control. M represents DNA marker. The percentage of mutation was calculated by Image-J. FIG. 4D shows single cell cloning of EB5 with desired single-base change in the gfp gene. The gfp fragments were PCR amplified from 12 cell lines obtained by single cell cloning and subjected to 2% agarose electrophoresis following digestion by BfaI restriction enzyme. Two desired cell lines, #4 and #6, have been obtained. FIG. 4E shows FACS analysis of fluorescence cell population 3 days after transfection of ssODN-2 and injection of TALEN proteins by P. aeruginosa. As a control, gfp silenced EB5 cells (EB5-Mut1) were injected of the TALEN proteins only. Percentage of the GFP-positive cells in the whole population are shown. FIG. 4F shows data related to transfection of ssODN-2 and TALEN protein injection into EB5-Mut1. GFP-positive cells were FACS-Sorted and a 350 bp fragment encompassing the TALEN-targeting region was amplified by PCR. The PCR products were digested with (+) or without (−) Sad restriction enzyme and subjected to 2% agarose electrophoresis. Uninfected EB5 cell and gfp silenced EB5 cell (EB5-Mut1) were used as controls. The percentage of mutation was calculated by Image-J.

FIGS. 5A-5H show T3SS mediated injection of TALEN proteins into human ESCs and iPSCs. FIG. 5A shows the percent reduction of GFP-positive LT2e-H9CAGGFP cells after infection by the PAK-JΔ8/pExoS54-FLAG-TALEN1 and PAK-JΔ8/pExoS54-FLAG-TALEN2 (1:1 ratio) at the indicated MOI for 3 hours. The data were compared with that of untreated control by two-sample t-test, **P.<0.001. Error bars represent standard deviations of triplicate assays. FIG. 5B shows fluorescence intensity of LT2e-H9CAGGFP cells transfected by eukaryotic expression plasmids encoding gfp-targeting TALEN pair or infected by a 1:1 mixture of PAK-JΔ8/pExoS54-FLAG-TALEN1 and PAK-JΔ8/pExoS54-FLAG-TALEN2. Cells were analyzed by flow cytometry 3 days after the treatments. The data were compared to that of untreated control by two-sample t-test, ***P<0.0001. Error bars represent standard deviations of triplicate assays. FIG. 5C shows a representative GFP-negative LT2e-H9CAGGFP cell cluster following bacterial delivery of gfp-targeting TALEN protein pair, observed under fluorescence microscope. FIG. 5D shows a schematic representation of TALEN binding sites on HPRT1 gene. The left and right TALEN binding sequences are shown in green and blue, respectively, and the spacer sequence is shown in red. The sequences, from top to bottom, correspond to SEQ ID NOs: 57-58. FIG. 5E shows sequence changes in the HPRT1 target site among iPSCs surviving the 6TG selection after P. aeruginosa mediated TALEN delivery. The sequences, from top to bottom, correspond to SEQ ID NOs: 59-64. FIG. 5F shows strategy of single-base modification in the HPRT1 gene. The 72 bp long ssODN-3 introduces a stop codon in the HPRT open reading frame while eliminating an XhoI restriction enzyme recognition site. The sequences, from top to bottom, correspond to SEQ ID NOs: 65, 19, and 66. FIG. 5G shows PCR amplification of the HPRT1 gene from iPSCs surviving the 6TG selection after gene modification by the ssODN-3 and P. aeruginosa mediated TALEN delivery. The DNA fragments were digested (+) or undigested (−) with XhoI before subjecting to electrophoresis on 0.8% agarose gel. Untreated iPSCs and iPSCs injected of the TALEN but without ssODN-3 template were used as negative controls. The percentage of mutation was calculated by Image-J. FIG. 5H shows sequence changes in the HPRT1 target site among iPSCs surviving the 6TG selection after P. aeruginosa mediated TALEN delivery and ssODN-3 transfection. The sequences, from top to bottom, correspond to SEQ ID NOs: 59 (wt), 67 (C/T), 68 (439), 69 (Δ24), and 70 (Δ20).

FIG. 6 shows a schematic illustration of T3SS mediated genome editing. ExoS54-TALEN fusion proteins are produced inside bacterial cells and directly injected into the host cytosol through the bacterial T3SS needle. The injected ExoS54-TALEN proteins target to nucleus, find their target sequences on the chromosome and introduce double stranded break (DSB). In the presence of ssDNA template (delivered by transfection), the DSB triggers homologous recombination, resulting in desired base changes on the chromosomally encoded gfp or hprt1 gene.

FIGS. 7A-7B show a cytotoxicity assay of various P. aeruginosa strains. FIG. 7A shows HeLa cells and mES cell line R1 were infected with indicated strains for 3 h at MOI of 100, and cells that remained adhered were counted. ΔSTY, deleted of all three type III secreted toxins; Δ8, deleted of 8 virulence genes. FIG. 7B shows mES cells were infected with the indicated strains for 2, 3, 4 h at an MOI of 100, and cells that remained adhered were counted. PAK-J, wild-type; Control, without bacterial infection. Data represent means of three replicate experiments. Error bars represent SD. *P<0.05, **0.01<P<0.05, ***0.001<P<0.01.

FIGS. 8A-8C show T3SS-dependent protein injection capability of various P. aeruginosa strains. FIG. 8A shows immunohistochemistry of HeLa cells following infection by ΔSTY/piExoS-Flag or Δ8/piExoS-Flag for 1, 2, 3, 4 h at MOI of 50. Cells were stained with anti-Flag antibody and nuclei with DAPI stain. FIG. 8B shows quantification of anti-Flag immunofluorescence staining intensity within HeLa cells as shown in FIG. 8A. Data represent means of three replicative experiments. Error bars represent SD. *P<0.05, **0.01<P<0.05. FIG. 8C shows immunohistochemistry of mES cells following infection by ΔSTY/piExoS-Flag or Δ8/piExoS-Flag and for 3 h at MOI of 50. Cells were stained with anti-Flag antibody; nuclei with DAPI stain. Bar=50 μm.

FIGS. 9A-9B show elimination of residual bacteria by antibiotic treatment. FIG. 9A shows mES cell line R1 was infected with Δ8 at MOI of 100 for 3 hours. Supernatants and adherent ES cells of each well were collected and serially diluted, then plated on LB-agar plates to enumerate the bacterial cell number (cfu/well) of planktonic bacteria and bacteria attached to the mES cells, respectively. FIG. 9B shows infection was terminated by washing cells with PBS and continuous growth of the mES cells on culture medium containing 20 μg/mL ciprofloxacin. After antibiotic treatment (time 0 h), 50-μL cell culture supernatant per well was used for LDH release assay. At the same time, mES cell colonies were scraped and lysed by 0.2% Triton-X100, the lysates were serially diluted and plated on LB-agar plates to calculate the residual bacterial numbers (cfu/well).

FIGS. 10A-10D show bacterial T3SS mediated production and injection of TF proteins into mES cells. FIG. 10A shoes a schematic representation of plasmids encoding the ExoS₅₄-Gata4, ExoS₅₄-Mef2c and ExoS₅₄-Tbx5 fusions with a Flag-tag fused in the middle. FIG. 10B shows ΔexsA, ΔpopD and Δ8 strains with plasmids expressing ExoS₅₄-Gata4, ExoS₅₄-Mef2c or ExoS₅₄-Tbx5 fusion with Flag tag fused in the middle. Each strain was examined for the ability to produce and secrete the fusion protein by anti-Flag immunoblot of the bacterial pellet and culture supernatant. FIG. 10C shows mESCs were infected with each strain at indicated MOI for 3 hours, lysed and examined for protein injection by anti-Flag immunoblot. FIG. 10D shows a schematic representation of T3SS-dependent protein secretion into the supernatant (in vitro secretion) or eukaryotic cells (protein translocation).

FIG. 11 shows TF delivery into mESCs. mESCs were infected with Δ8/Gata4, Δ8/Mef2c, Δ8/Tbx5 respectively, for 3 hours at MOI 50 and subsequently fixed and immunostained with anti-Flag to illuminate translocated ExoS₅₄-Flag-TF proteins. Nuclei were stained with DAPI. Bar is 100 ΔM.

FIG. 12 shows subcellular localization of injected TFs Immunohistochemistry of HeLa cells following infection by Δ8/piExoS-Flag, Δ8/pExoS₅₄F-Gata4, Δ8/pExoS₅₄F-Mef2c or Δ8/pExoS₅₄F-Tbx5 (4 h at MOI 50). Cells were stained with anti-Flag antibody; nuclei were stained with DAPI. Bar=50 μm.

FIGS. 13A-13B show intracellular stability of injected proteins. mES cells were infected with Δ8/pExoS54F-Gata4, 48/pExoS₅₄F-Mef2c and Δ8/pExoS₅₄F-Tbx5 at MOI of 50 for 3 hours, respectively. FIG. 13A shows post bacterial infection (time 0 h), nuclear proteins were extracted at the indicated time and subjected Western blot. Anti-Flag antibody was used to detect the injected TF fusion, anti-Oct3/4 antibody was used to detect endogenous TF Oct3/4. FIG. 13B shows quantification of Western blots by Image J, half-life (t_{1/2}) was determined by time vs. injected protein curve.

FIGS. 14A-14C show GMT delivery promotes de novo differentiation of ESC-CMs. FIG. 14A shows protocol for differentiation of cardiomyocytes from embryoid bodies (EBs). mESCs were dissociated into single cells on day-0 and cultured in suspension for 2 days in hanging drops and then plated on gelatin coated culture plate. FIG. 14B shows GMT injection at various MOI on day-5, and total GFP fluorescence of each EB were measured on day-12. FIG. 14C shows live cell images showing αMHC-GFP⁺ cardiomyocytes in 12-day old EBs.

FIGS. 15A-15B show determination of an optimal ratios of GMT for cardiomyocyte differentiation. FIG. 15A show response surface plots showing effects of various parameters on fluorescence intensity of EBs and contour plots showing predicted optimal response. FIG. 14B shows total fluorescence per EB (TF/EB) following injection of GMT at the relative ratios before and after optimization.

FIGS. 16A-16C shows multiple rounds of GMT delivery improves ESC-CMs differentiation. FIG. 16A shows GMT injection at MOI=40G:10M:25T for one time on day-5 or 3 times on days-5, 7 & 9, then TF/EB were recorded on day-12. Data represents mean ±SD, (n>20); **0.01<P<0.05, ***0.001<P<0.01. FIG. 16B shows percentage of EBs containing beating areas. More than 40 embryoid bodies were counted per condition per day (48 EBs per condition in total). NC, negative control (EBs without any treatment). FIG. 16C shows live cell images showing GFP⁺ contraction cluster of 12-day old EBs.

FIG. 17 shows relative expression levels of cardiac marker genes. EBs with three rounds of GMT delivery (GMT) or non GMT-treated control (NC) were subjected to quantitative PCR analysis and normalized to the mES cells. Endogenous GMT, cardiac mesodermal markers NKX2.5 and dHAND, and cardiomyocyte marker MYH6. Red arrows indicate the days of GMT delivery. Error bars represent SEM of 3 biological replicates. *P<0.05.

FIGS. 18A-18G show the additive effect of Activin A on the ESC-CMs differentiation promoted by the GMT deliveries. FIG. 18A shows Activin A treatment on day-2, and GFP fluorescence intensity measurements of mesodermal marker Brachury-GFP on day-4. FIG. 18B shows fluorescence intensities of EBs with or without GMT injection in the presence or absence of Activin A (30 ng/mL) in culture medium. FIG. 18C shows quantitative PCR measurements of Brachury on EB day-5. FIG. 18D shows fluorescence-activated cell sorting (FACS) analysis of αMHC-GFP positive cells in 12 day-old EBs. FIG. 18E shows quantitative PCR measurements of Nkx2.5 and αMHC in EB on day-12. NC, negative control; GMT, 3 rounds of GMT delivery; GMT+Activin, 3 rounds of GMT delivery plus 30 ng/mL Activin A pre-treated for 3 days. Data represents mean ±s.e.m., (n>3); *P<0.05, **0.01<P<0.05, ***0.001<P<0.01. FIG. 18F shows live cell images showing αMHC-GFP⁺ cardiomyocytes of 12-day old EBs. Controls were spontaneously differentiated EBs. Representative FACS analysis and the percentage of ESC derived αMHC-GFP⁺ cardiomyocytes. FIG. 18G shows a protocol for differentiation of cardiomyocytes in EB system with Activin A treatment and GMT delivery. FIGS. 19A-19B show characterizations of GMT induced ESC-CMs. FIG. 19A shows single cells dissociated from day-12 EBs were stained with anti-cardiac actin (i), anti-sarcomeric α-actinin (ii) and anti-cardiac troponin T (iii). Nuclei are stained with DAPI (blue). FIG. 19B shows contractile movement analysis demonstrating functional expression and integration of β-adrenergic and muscarinic signaling in ESCs-derived cardiomyocytes with rhythmic contractile movement. The magnitude and frequency of contraction increased after administration of the β-adrenergic agonist isoproterenol (ISO). Subsequent application of carbachol led to a blockage of the ISO effect.

FIG. 20 shows a schematic representation of directed differentiation of ES cells into CMs by bacterial injection of transcription factors.

### DETAILED DESCRIPTION OF INVENTION

Aspects of the disclosure relate to the delivery of proteins, such as genome editing proteins, to target cells using Pseudomonas bacteria that are deficient in exoS, exoT, exoY, and popN activity, and that further lack at least xcpQ, lasR-I, rhlR-I, and/or ndk activity. Pseudomonas aeruginosa is naturally able to deliver a series of proteins into host cells via its type III secretion system (T3SS). This capability makes T3SS a potentially useful tool for the delivery of exogenous proteins into mammalian cells. However, certain proteins (for example large proteins) are not effectively delivered to certain cell types (e.g., stem cells). In some aspects, the disclosure relates to the surprising discovery that certain genetically engineered bacteria are capable of effectively delivering proteins to certain mammalian cells without the cytotoxicity previously associated with the bacterial delivery of proteins. Compositions and methods described herein are useful for the delivery of proteins into sensitive cell types, for example stem cells (e.g., embryonic stem cells (ESCs) and pluripotent stem cells (PSCs)).

Accordingly, in some aspects the disclosure provides a genetically modified bacterium for improved delivery of proteins into mammalian cells. In some embodiments, the bacterium is a Pseudomonas bacterium deficient in (e.g., lacking) exoS, exoT, exoY, and popN proteins, further deficient in (e.g., lacking) at least one of the following proteins xcpQ, lasR-I, rhlR-I, and ndk. In some embodiments, the bacterium comprises a polynucleotide encoding a fusion protein, wherein the fusion protein comprises a heterologous protein fused to a bacterial secretion domain.

Pseudomonas aeruginosa is a Gram-negative opportunistic pathogen that possesses a Type III secretion system (T3SS). Generally, some bacteria utilize T3SS to inject toxic effector proteins into mammalian host cells. The effectors secreted by the T3SS of P. aeruginosa—exoenzymes S, T, Y and U (ExoS, ExoT, ExoU, and ExoY)—are the major contributors to acute toxicity during the course of an infection. The majority of P. aeruginosa isolates encode three of the four T3SS effectors, either STY or UTY. The amino acid sequences of ExoS, ExoT, ExoY, and ExoU are represented by SEQ ID NO: 1-4, respectively. As used herein, a “Pseudomonas bacterium deficient in exoS, exoT, exoY, and popN proteins” refers to a Pseudomonas bacterium that is defective (e.g., has a lower expression level or activity, or lacks a gene or a portion thereof) for the exoenzymes S, T and Y, and the negative regulator of T3SS, popN. As used herein, a “ΔSTYN Pseudomonas bacterium” refers to a Pseudomonas bacterium that lacks the exoenzymes S, T and Y, and the negative regulator of T3SS, popN.

The instant disclosure is based, in part, on the recognition that a Pseudomonas bacterium lacking other virulence factors and/or T3SS effectors may be capable of delivering proteins into mammalian cells with less cytotoxicity than Pseudomonas bacteria not lacking these genes. Examples of virulence factors include but are not limited to the xcpQ, lasR-I, rhlR-I, and ndk. In some embodiments, the virulence factor is xcpQ encodes type II protein secretion system protein D (also referred to as general secretion pathway protein D). A non-limiting example of an xcpQ virulence factor is represented by NCBI Gene ID 880114. In some embodiments, the virulence factor is lasR or lasI (also referred to as lasR-I), which are components of N-acyl homoserine lactone (AHL)-dependent quorum sensing (QS) system. A non-limiting example of a lasR-I virulence factor is represented by GenBank Accession No. EU074852.1. In some embodiments, the virulence factor is rhl R or rhlI (also referred to as rhlR-I), which are components of N-acyl homoserine lactone (AHL)-dependent quorum sensing (QS) system. A non-limiting example of a rhlR-I virulence factor is represented by GenBank Gene ID: 878968. In some embodiments, the virulence factor is nucleoside diphosphate kinase (ndk), which is a Type III secreted effector protein (e.g., toxin). A non-limiting example of an ndk virulence factor is represented by GenBank Gene ID: 879892.

Thus, in some embodiments, the disclosure provides a Pseudomonas bacterium deficient in exoS, exoT, exoY, and popN proteins (e.g., ASTYN Pseudomonas bacterium) lacking one or more genes selected from the group consisting of: xcpQ, lasR-I, rhlR-I, and/or ndk. In some embodiments, the disclosure provides a Pseudomonas bacterium deficient in exoU, exoT, exoY, and popN proteins (e.g., AUTYN Pseudomonas bacterium) lacking one or more genes selected from the group consisting of: xcpQ, lasR-I, rhlR-I, and/or ndk. The xcpQ gene is associated with bacterial type II secretion systems. The lasR-I and rhlR-I genes are associated with quorum sensing. The ndk gene encodes the T3SS effector nucleoside diphosphate kinase (NDK). In some embodiments, the 4STYN Pseudomonas bacterium lacks xcpQ, lasR-I, rhlR-I, and ndk. A ΔSTYN Pseudomonas bacterium lacking xcpQ, lasR-I, rhlR-I, and ndk can also be referred to as a “Δ8 Pseudomonas bacterium”.

The deficiency of exoenzyme activity and regulatory activity in Pseudomonas bacterium deficient in exoS, exoT, exoY, and popN (e.g., ΔSTYN Pseudomonas), and/or deficient in one or more of xcpQ, lasR-I, rhlR-I, and/or ndk (e.g., Δ8 Pseudomonas) can be caused by a variety of genetic alterations, for example chromosomal deletions, mutations (e.g., nonsense mutations, missense mutations, frameshift mutations, point mutations, non-conservative substitution, or a combination thereof in each of the affected genetic loci). Genetic alterations can be made to an entire gene (e.g., deletion of a gene from a chromosome) or a portion of a gene (e.g., a deletion of a gene fragment or domain) In some embodiments, the ΔSTYN Pseudomonas or Δ8 Pseudomonas are produced by deletion of the exoS, exoT, exoY, popN, xcpQ, lasR-I, rhlR-I, and ndk genes in their entirety. In some embodiments, genetic alterations can be transient (e.g., knockdown or RNAi) or stable (e.g., deletion of a gene from a chromosome).

Any suitable Pseudomonas bacterium can be genetically altered to become a ΔSTYN Pseudomonas bacterium and/or a Δ8 Pseudomonas bacterium. There are at least 140 species of Pseudomonas including Pseudomonas abietaniphila; P. agarici; P. agarolyticus; P. alcaliphila; P. alginovora; P. andersonii; P. antarctica; P. asplenii; P. azelaica; P. batumici; P. borealis; P. brassicacearum; P. chloritidismutans; P. cremoricolorata; P. diterpeniphila; P. filiscindens; P. frederiksbergensis; P. gingeri; P. graminis; P. grimontii; P. halodenitrificans; P. halophila; P. hibiscicola; P. hydrogenovora; P. indica; P. japonica; P. jessenii; P. kilonensis; P. koreensis; P. lini; P. lurida; P. lutea; P. marginata; P. meridiana; P. mesoacidophila; P. pachastrellae; P. palleroniana; P. parafulva; P. pavonanceae; P. proteolyica; P. psychrophila; P. psychrotolerans; P. pudica; P. rathonis; P. reactans; P. rhizosphaerae; P. salmononii; P. thermaerum; P. thermocarboxydovorans; P. thermotolerans; P. thivervalensis; P. umsongensis; P. vancouverensis; P. wisconsinensis; P. xanthomarina; and P. xiamenensis. Non-limiting examples of Pseudomonas bacteria groups and species include: P. aeruginosa group: P. aeruginosa; P. akaligenes; P. anguilliseptica; P. citronellolis; P. flavescens; P. jinjuensis; P. mendocina; P. nitroreducens; P. oleovorans; P. pseudoalcaligenes; P. resinovorans; P. straminae; P. chloroaphis group: P. aurantiaca; P. chlororaphis; P.s fragi; P. lundensis; P. taetrolens; P. fluorescens group: P. azotoformans; P. brenneri; P. cedrina; P. congelans; P. corrugata; P. costantinii; P. extremorientalis; P. fluorescens; P. fulgida; P. gessardii; P. libanensis; P. mandelii; P. marginalis; P. mediterranea; P. migulae; P. mucidolens; P. orientalis; P. poae; P. rhodesiae; P. synxantha; P. tolaasii; P. trivialis; P. veronii; P. pertucinogena group: P. denitrificans; P. pertucinogena P. putida group: P. fulva; P. monteilii; P. mosselii; P. oryzihabitans; P. plecoglossicida; P. putida; P. stutzeri group: P. balearica; P. luteola, P. stutzeri; P. syringae group: P. avellanae; P. cannabina; P. caricapapyae; P. cichorii; P. coronafaciens; P. fuscovaginae; P. tremae; P. viridiflava.

In some embodiments, other bacteria having T3SS may be used. For example, species of Shigella, Salmonella, Escherichia coli, Vibrio, Burkholderia, Yersinia, and Chlamydia also possess T3SS and T3SS effector proteins that may be useful for the delivery of proteins into cells. For example, a non-Pseudomonas bacterium may include a secreted effector protein having a secretion signal sequence having structural homology to Pseudomonas ExoS, functional homology to Pseudomonas ExoS, sequence homology to Pseudomonas ExoS, or any of the foregoing. Non-Pseudomonas bacteria can also have distinct secretion signal sequences that are not homologs of Pseudomonas secretion signals but function in a similar manner.

In some aspects, the disclosure relates to the delivery of fusion proteins into cells via a Type III secretion system (T3SS). As used herein, the term “Type III secretion system” refers to a protein delivery mechanism found in several Gram negative bacteria that includes a needle (e.g., injectosome) anchored to a membrane-integral basal body. Generally, the needles are inserted into the host cell membrane and inject the protein effector molecules. Injection of bacterial effectors into host cells results in a various physiological changes, ranging from morphological alteration (e.g., to facilitate or block invasion) to killing of the host cells (e.g., by immune cells), all of which provide the bacterial pathogen with a survival advantage within the host environment. The structure and function of T3SS are disclosed, for example in Hueck (1998), Type III protein secretion systems in bacterial pathogens of animals and plants, Microbiol Mol Biol Rev, 62(2), pp. 379-433.

Bacteria can be genetically modified to deliver heterologous proteins to mammalian cells via T3SS. Thus, in some embodiments, the T3SS delivers a fusion protein. As used herein, the term “fusion protein” refers to a non-naturally occurring protein comprising a first domain from a first protein or first peptide contiguously linked to a second domain of a second protein or second peptide. As used herein, the term “linked” refers to the joining of two polypeptides via one or more covalent bonds (e.g., peptide bonds). Fusion proteins may also comprise a protein or protein domain linked to a secretion domain or signal. Secretion signals or signal peptides are generally located at the N-terminus of a protein and direct trafficking of said protein out of a cell. However, in some embodiments, a secretion signal or signal peptide is located at the C-terminus of a protein. Generally, T3SS require the presence of a secretion signal for the export of a protein. Therefore, in some embodiments, a fusion protein comprises a bacterial secretion domain In some embodiments, the bacterial secretion domain is a T3SS secretion domain, for example those disclosed by Bichsel et al. (2001), Bacterial delivery of nuclear proteins into pluripotent and differentiated cells, PLoS ONE, 6: e16465. For example, the N-terminal sequences of ExoS protein may direct the secretion of proteins via T3SS. Therefore in some embodiments, the bacterial secretion domain is an ExoS secretion domain. ExoS secretion domains are generally referred to by the number of amino acids they contain. For example, ExoS54 refers to the first 54 amino acids at the N-terminus of ExoS. ExoS secretion domains include but are not limited to ExoS17 (SEQ ID NO: 5), ExoS54 (SEQ ID NO: 6), ExoS96 (SEQ ID NO: 7), and ExoS234 (SEQ ID NO: 8). In some embodiments, ExoS protein is Pseudomonas ExoS protein. In some embodiments, the secretion domain is represented by SEQ ID NO: 6. Other N-terminal sequences of T3SS effector proteins can be used. In some embodiments, the secretion domain is one of the following secretion domains: an ExoT secretion domain, an ExoU secretion domain, or an ExoY secretion domain.

In some embodiments, the fusion protein comprises a bacterial secretion domain linked to a heterologous protein. As used herein, the term “heterologous protein” refers to any protein that is not naturally present in the bacterium. Examples of heterologous proteins include but are not limited to peptide antigens, receptors, antibodies and enzymes (e.g., kinases, nucleases, etc.).

In some aspects, the disclosure relates to the surprising discovery that bacterial T3SS can be used to deliver a protein or proteins that induce cell differentiation. Generally, the cell or cells that are differentiated are stem cells (e.g., embryonic stem cells or pluripotent stem cells, e.g., induced pluripotent stem cells). Other cell types (e.g., fibroblasts) can also be induced to differentiate.

As used herein, the term “transcription factor” refers to a protein which binds to a DNA regulatory region of a gene to control the synthesis of mRNA. Without wishing to be bound by any particular theory, transcription factors regulate expression of genes involved in signaling cascades that result in cell differentiation. For example, differentiation of stem cells to cardiomyocytes is regulated by the transcription factors Gata4, Mef2c, and Tbx5. In another example, differentiation of embryonic stem cells (ESCs) into thyroid cells is regulated by the transcription factors NKX2-1 and PAX8. In another example, differentiation of ESCs into hepatocytes is regulated by the transcription factors GATA4, FOXa3, and Hinfla. In some embodiments, one or more transcription factors (e.g., as described herein) can be delivered by a modified bacterium for the purposes of differentiating stem cells as described herein.

In some embodiments, the protein or proteins that induce(s) cell differentiation are selected from the group consisting of Gata4, Mef2c, and Tbx5. In some embodiments, the protein or proteins that induce(s) cell differentiation is a fusion protein comprising Gata4, Mef2c, or Tbx5 and a bacterial secretion domain (e g., ExoS54). For example, in some embodiments, the protein or proteins that induce(s) cell differentiation is selected from the group consisting of ExoS54-Gata4, ExoS54-Mef2c, and ExoS54-Tbx5. In some embodiments, a genetically modified bacterium comprises a polynucleotide encoding a single fusion protein encoding a transcription factor (e.g., ExoS54-Gata4, ExoS54-Mef2c, or ExoS54-Tbx5). In some embodiments, a genetically modified bacterium comprises a polynucleotide or polynucleotides encoding multiple (e.g., 2, 3, 4, 5, or more than 5) transcription factors (e.g., in the form of fusion proteins, for example ExoS54-Gata4, ExoS54-Mef2c, and ExoS54-Tbx5).

In some aspects, the instant disclosure relates to the surprising discovery that bacterial T3SS can be used to deliver large proteins into mammalian cells. Bacteria described herein may have the capability to deliver large proteins, for example genome editing proteins (e.g., TALENs and/or CRISPR/Cas proteins), to mammalian cells. One example of a TALEN that targets gfp is represented by SEQ ID NO: 26. An example of a Cas protein (e.g., Cas9) is represented by SEQ ID NO: 27. Other examples of genome editing proteins include Zinc Finger Nucleases (ZFNs) and engineered meganuclease re-engineered homing endonucleases. In some embodiments, a genome editing protein (e.g., a TALEN or a CRISPR/Cas protein) is fused to a bacterial secretion signal (e.g., ExoS54) to enable delivery of the genome editing protein to a cell via a bacterial secretion system. Examples of a TALEN protein fused to a bacterial secretion signal and a CRISPR/Cas protein fused to a bacterial secretion signal are represented by SEQ ID NOs: 28 and 29, respectively. In some embodiments, the fusion protein delivered by the modified bacterium is larger than 50 kDa. In some embodiments, the fusion protein delivered by the modified bacterium is larger than 75 kDa. In some embodiments, the fusion protein delivered by the modified bacterium is larger than 100 kDa. In some embodiments, the fusion protein delivered by the modified bacterium is larger than 150 kDa. In some embodiments, the fusion protein delivered by the modified bacterium is up to 200 kDa.

In some embodiments, a bacterium described herein is capable of delivering proteins into a variety of cell types. For example, protein may be delivered to epithelial cells, endothelial cells, CNS cells (e.g., neurons, glial cells, etc.), organ cells (e.g., kidney cells, cardiac cells, lung cells, etc.), structural cells (e.g., extracellular matrix cells), germ cells, blood cells, immune cells (e.g., T cells, dendritic cells, etc.) and stem cells. In some embodiments, the cell is a stem cell. Any stem cells may be used, such as embryonic stem cells, adult stem cells, induced pluripotent stem cells, hematopoietic stem cells, mesenchymal stem cells, or neuronal stem cells. In some embodiments, the stem cell is a mammalian stem cell. In some embodiments, the stem cell is a human stem cell. In some embodiments, the human stem cell is a human embryonic stem cell (hESC) or human induced pluripotent stem cell (hiPSC). In some embodiments, the cell is a fibroblast, or a human fibroblast.

In some aspects, the disclosure relates to improved methods for delivering protein into cells. In some embodiments, the disclosure provides a method for a method of delivering one or more proteins into one or more isolated cells, comprising: incubating the cell or cells with a Pseudomonas bacterium deficient in exoS, exoT, exoY, and popN proteins (e.g., a ASTY Pseudomonas bacterium), wherein the bacterium is deficient in one or more genes selected from the group consisting of: xcpQ, lasR-I, rhlR-I, and/or ndk, said bacterium comprising a polynucleotide encoding a fusion protein, wherein the fusion protein comprises a heterologous protein fused to a bacterial secretion domain; and incubating the isolated infected cell or cells for a period of time sufficient to deliver the one or more proteins into said cell or cells.

Methods described by the disclosure may be used to deliver proteins into cells for a variety of purposes, such as delivery of therapeutic proteins, or genome editing. As used herein, “genome editing” refers to the adding, disrupting or changing the sequence of specific genes by insertion, removal or mutation of DNA from a genome using artificially engineered proteins and related molecules. For example, genome editing proteins, such as TALENS, may be delivered to a cell using a method described herein. The TALEN can introduce double stranded breaks at a target locus in the host cell genome, resulting in altered gene function and/or expression. TALENS can also promote DNA repair (e.g., non-homologous end joining or homology-directed repair), which is useful for rescue construct-mediated stable integration of foreign genetic material into the genome of a host cell. For example, in a gene therapy context, a TALEN can be used to cleave a DNA sequence having a disease-causing mutation at a locus containing the mutation. A non-mutant nucleic acid which repairs the cleaved mutant DNA (e.g., by non-homologous end joining or homology directed repair) can then be provided by a rescue construct in order to restore normal gene function. Rescue constructs, comprising a polynucleotide encoding a desired insertion or mutation, can be delivered before, after, or simultaneously to a genome editing protein in order to introduce a mutation or other alteration at the target locus. A rescue construct can be single-stranded polynucleotide or a double-stranded polynucleotide. In some embodiments, a rescue construct is a single-stranded oligonucleotide DNA (ssODN). In some embodiments, a rescue construct is a plasmid, viral vector, or interfering RNA (dsRNA, siRNA, shRNA, miRNA, AmiRNA, etc.). The one or more isolated cells can be transfected with a rescue construct before, after or simultaneous to contact with the bacterium. In some embodiments, the rescue construct is delivered separately from the bacterium. In some embodiments, the rescue construct is expressed by the bacterium.

In some aspects, the disclosure relates to the surprising discovery that genetically modified bacteria can deliver multiple transcription factors via T3SS to a cell or cells, thereby inducing the differentiation of the cell or cells. Accordingly, in some embodiments the disclosure provides a method for inducing differentiation of one or more cells to cardiomyocytes, the method comprising: incubating the cell or cells with a first modified Pseudomonas bacterium; incubating the cell or cells with a second modified Pseudomonas bacterium; and, incubating the cell or cells with a third modified Pseudomonas bacterium, wherein the first bacterium expresses Gata4 or a Gata4 fusion protein (e.g., ExoS54-Gata4), the second bacterium expresses Mef2c or a Mef2C fusion protein (e.g., ExoS54-Mef2c), and the third bacterium expresses Tbx5 or a Tbx5 fusion protein (e.g., ExoS54-Tbx5). In some embodiments of the method, the cell or cells are selected from the group consisting of stem cell(s) and fibroblast(s).

Absolute multiplicity of infection (MOI) is a parameter used to express the ratio of infectious agents to infection targets. For example, an absolute MOI of 10 indicates that in a given area (e.g., a volume of culture media) there are 10 infectious agents (e.g., bacteria) for a given target of infection (e.g., a cell). Generally, absolute MOI is an important variable to consider for effective gene or protein delivery. In some embodiments, the absolute MOI of Pseudomonas bacteria to target cells ranges from 10-1000. In some aspects, the disclosure is based upon the recognition that the relative MOI ratio of bacteria configured for delivering transcription factors (e.g., Gata4, Mef2c, and Tbx5) to cells (e.g., the MOI) significantly affects the differentiation of the cells to which the transcription factors are delivered. Relative MOI ratio refers to the proportion of absolute MOI for each bacterial delivery strain (e.g., absolute MOI of bacteria delivering Gata4 : absolute MOI of bacteria delivering Mef2c : absolute MOI of bacteria delivering Tbx5). In some embodiments, the relative MOI ratio of each bacterial delivery strain ranges from about 1 to about 100. In some embodiments, the relative MOI ratio of bacteria expressing Gata4 or a Gata4 fusion protein (e.g., ExoS54-Gata4): bacteria expressing Mef2c or a Mef2c fusion protein (e.g., ExoS54-Mef2c): bacteria expressing Tbx5 or a Tbx5 fusion protein (e.g., ExoS54-Tbx5) ranges from about 1:1:1 to about 4:1:2.5.

Proteins delivered to a cell or cells are generally degraded by cellular machinery. The presence of a protein delivered into a cell can be measured by half-life. As used herein, the term “half-life” refers to the amount of time that elapses between the delivery of a protein to a cell and degradation of half the protein delivered to the cell. The average half-life of a protein delivered to a cell can range from about 0.5 hours to about 24 hours. In some embodiments, the average half-life of a protein delivered to a cell or cells ranges from about 1 hour to about 10 hours. In some embodiments, the average half-life of a protein delivered to a cell or cells ranges from about 3 hours to about 7 hours. In some embodiments, the average half-life of a protein delivered to a cell or cells is about 1, 1.5, 2, 2.5, 3, 3.5, 4, 4.5, 5, 5.5, 6, 6.5, 7, 7.5, 8, 8.5, 9, 9.5, or 10 hours. However, proteins having longer or shorter half-lives can be used.

In some cases, it is desirable for a protein delivered to a cell or cells to remain active in the cell or cells for a period of time longer than the protein half-life. Accordingly, multiple deliveries of proteins by genetically modified bacteria are also contemplated by the disclosure. In some embodiments, a protein or proteins are delivered to a cell or cells (e.g., by incubating the cell or cells with the genetically modified bacteria) between 2 and 10 times. In some embodiments, a protein or proteins are delivered to a cell or cells 2, 3, 4, 5, 6, 7, 8, 9, or 10 times. The skilled artisan recognizes that the cell or cells to which the protein or proteins are delivered can be washed between delivery of protein by genetically modified bacteria.

Cardiac development is a dynamic process that is tightly orchestrated by the sequential expression of multiple signal transduction proteins and transcription factors working in a combinatory manner. Generally, three main steps occur to generate cardiomyocytes from pluripotent stem cells: (i) mesoderm induction and patterning, (ii) cardiac specification, and (iii) cardiomyocyte maturation. Transforming growth factor (TGF) β-family member Nodal efficiently induces mesoderm. In some embodiments, methods described by the disclosure further comprise incubating the cell or cells with a mesoderm inducer (e.g., Nodal or Activin A).

In some embodiments, the mesoderm inducer is added to the growth medium of cells. The disclosure is based, in part, on the recognition that Activin A, which signals through many of the same downstream pathways as Nodal, shows an additive effect on stem cell differentiation promoted by T3SS delivery of Gata4, Mef2c and Tbx5. In some embodiments, the cell or cells are incubated with Activin A. In some embodiments, Activin A (or other growth factor) is added along with the modified bacteria. In some embodiments, Activin A (or other growth factor) is added after the modified bacteria.

The concentration or amount of Activin A incubated with the cell or cells can range from about 1 ng/mL to about 60 ng/mL. In some embodiments, the amount of Activin A incubated with the cell or cells ranges from about 3 ng/mL to about 30 ng/mL. In some embodiments, the amount of Activin A incubated with the cell or cells ranges from about 10 ng/mL to about 50 ng/mL. In some embodiments, the amount of Activin A incubated with the cell or cells is about 3 ng/mL, 5 ng/mL, 10 ng/mL, 15 ng/mL, 16 ng/mL, 17 ng/mL, 18 ng/mL, 19 ng/mL, 20 ng/mL, 21 ng/mL, 22 ng/mL, 23 ng/mL, 24 ng/mL, 25 ng/mL, 26 ng/mL, 27 ng/mL, 28 ng/mL, 29 ng/mL, 30 ng/mL, 31 ng/mL, 32 ng/mL, 33 ng/mL, 34 ng/mL, 35 ng/mL, 36 ng/mL, 37 ng/mL, 38 ng/mL, 39 ng/mL, 40 ng/mL, 41 ng/mL, 42 ng/mL, 43 ng/mL, 44 ng/mL, 45 ng/mL, 46 ng/mL, 47 ng/mL, 48 ng/mL, 49 ng/mL, or 50 ng/mL.

In some embodiments, the cell or cells are incubated with Nodal. The concentration or amount of Nodal incubated with the cell or cells can range from about 1 ng/mL to about 60 ng/mL. In some embodiments, the amount of Nodal incubated with the cell or cells ranges from about 3 ng/mL to about 30 ng/mL. In some embodiments, the amount of Nodal incubated with the cell or cells ranges from about 10 ng/mL to about 50 ng/mL. In some embodiments, the amount of Nodal incubated with the cell or cells is about 3 ng/mL, 5 ng/mL, 10 ng/mL, 15 ng/mL, 16 ng/mL, 17 ng/mL, 18 ng/mL, 19 ng/mL, 20 ng/mL, 21 ng/mL, 22 ng/mL, 23 ng/mL, 24 ng/mL, 25 ng/mL, 26 ng/mL, 27 ng/mL, 28 ng/mL, 29 ng/mL, 30 ng/mL, 31 ng/mL, 32 ng/mL, 33 ng/mL, 34 ng/mL, 35 ng/mL, 36 ng/mL, 37 ng/mL, 38 ng/mL, 39 ng/mL, 40 ng/mL, 41 ng/mL, 42 ng/mL, 43 ng/mL, 44 ng/mL, 45 ng/mL, 46 ng/mL, 47 ng/mL, 48 ng/mL, 49 ng/mL, or 50 ng/mL.

In some cases, it may be desirable to purify (e.g., separate) differentiated cells (e.g., cardiomyocytes) from other components, such as culture media, undifferentiated stem cells, and contaminants (e.g., genetically modified bacteria). Various suitable methods for separation of differentiated cells are known. For example, differentiated cells can be separated by mechanical (e.g., mechanical filtration) or biophysical (e.g., chromatography) methods. In some embodiments, a cell or cells (e.g., mammalian cells) that have been incubated with genetically modified bacteria are contacted with an antibiotic specific for the bacteria but not for the cells to which protein has been delivered. For example, a cell or cells can be contacted with one of the following antibiotics: ciprofloxacin, tobramycin, gentamicin, tetracycline, and carbenicillin.

### EXAMPLES

### Example 1

### T3SS-Mediated Delivery of Genome Editing Proteins

Pseudomonas aeruginosa is a common gram-negative opportunistic human pathogen which injects proteineous exotoxins directly into host cells via a type III secretion system (T3SS). The T3SS is a complex, needle-like structure on bacterial surface, responsible for the secretion of four known exotoxins: ExoS, ExoT, ExoY and ExoU. ExoS is best characterized for its functional domains, with its N-terminal sequence serving as a signal for injection. The N-terminal 54 amino acids of ExoS (ExoS54) can be used for delivery of the exogenous protein into mammalian cells. Transcription activator-like effector nuclease (TALEN) proteins fused with the ExoS54 can be injected into HeLa cells, achieving site specific DNA cleavage without the introduction of foreign genetic materials.

TALEN is a novel gene editing tool, which can specifically recognize target sequence as a dimer and introduce a double-strand DNA break (DSB) on the target site, triggering non-homologous end joining or homologous recombination. In the absence of homologous template, the DSB activates host DNA repair system, resulting in high frequency gene mutation, such as nucleotide mismatches, insertions or deletions. However, in the presence of a homologous template, the DSB triggers homologous recombination, introducing desired DNA sequence substitutions on target sites. Current methods of TALEN delivery utilize the introduction of foreign genetic materials, such as viral DNA/RNA, plasmid DNA or mRNA, making it difficult to meet safety requirements for biomedical applications.

Pluripotent stem cells (PSCs), such as embryonic stem cells (ESCs) and induced pluripotent stem cells (iPSCs), can be differentiated into a wide variety of cell and tissue types in vitro. Thus, gene editing in PSCs could be used to correct the root causes and thereby eliminate symptoms associated with genetic diseases. Accordingly, technologies capable of editing genes in PSCs are extremely important. To date, TALEN technology has been successfully applied to create disease models in many organisms, such as zebrafish, mice, rats, and human iPSCs (hiPSCs). Unfortunately, introduction of TALEN-encoding plasmid DNA results in low frequencies of DSB on the target sites and also poses a serious safety risk of insertional mutagenesis. As a result, it is critical to develop a highly efficient alternative method of introducing gene editing enzymes into the stem cells. This example describes the application of bacterial protein delivery technology to introduce TALEN proteins directly into mESCs, hESCs and hiPSCs. Data show that bacterial T3SS-mediated TALEN protein delivery into PSCs induces highly efficient target gene modifications with added benefits over the conventional plasmid transfection method.

### Materials and Methods

### Bacterial Strains and Plasmids

Strains and plasmids used in this example are listed in Table 1. Pseudomonas aeruginosa strains, PAK-JΔSTY is deleted of the type III secreted exotoxins (exoS, exoT and exoY) in the background of PAK-J; PAK-JΔpopD is deleted of popD, encoding a pore-forming protein required for the type III injection, in the background of PAK-J; and PAK-JΔ8 is deleted of popN, xcpQ, lasI, rhII and ndk in the background of PAK-JΔSTY. All P. aeruginosa strains were cultured in Luria Broth (LB) or LB agar plates at 37° C. Carbenicillin was used at a final concentration of 150 μg per ml for plasmid selection in P. aeruginosa.

TALENs targeting gfp and HPRT1 genes were constructed following the instruction of Golden Gate Cloning Kit from Voytas Laboratory. The left and right arm sequences of TALEN targeting gfp are 5′-TTCACCGGGGTGGTGCC-3′ and 5′-CTGGACGGCGACGTAAA-3′, (SEQ ID NOs: 9 and 10), respectively; the left and right arm sequences of TALEN targeting HPRT1 are 5′-GTAGGACTGAACGTCTTGCTC-3′ and 5′-GATGGGAGGCCATCACATTGT-3′, (SEQ ID NOs: 11 and 12), respectively. The ExoS54-Flag-TALEN fusion constructs were generated by in-frame fusion of the TALEN coding sequence to the pExoS54-Flag which had previously been described. The TALEN targeting region (350 bp) within gfp gene was amplified using PCR primers of gfp-Forward: 5′-CCTACAGCTCCTGGGCAACGTGCTGG-3′; and gfp-Reverse: 5′-CTGGACGTAGCCTTCGGGCATGGCGG-3′ (SEQ ID NOs: 13 and 14, respectively), while the TALEN targeting region (625 bp) within HPRT1 gene was amplified using PCR primers of HPRT1-Forward: 5′-TTTTGAGACAAGGTCTTGCTCTATTG-3′; and HPRT1-Reverse: 5′-CAGTATTGGCTTTGATGTAAAGTACT-3′ (SEQ ID NOs: 15 and 16, respectively). The PCR products were either subjected to digestion by restriction enzymes or directly cloned into pGEM-T Easy (Promega) vector and subjected to sequencing analysis.

Three 72 nucleotide long single-stranded donor template DNAs used to introduce desired nucleotide changes in either gfp or hprt1 gene through homologous recombination were ssODN-1: 5′-AGGAGCTGTTCACCGGGGTGGTGCCCATCCTGGTCTAGCTGGACGGCGACGTAAAC GGCCACAAGTTCAG CG-3′ (SEQ ID NO: 17), ssODN-2: 5′-AGGAGCTGTTCACCGGGGTGGTGCCCATCCTGGTCGAGCTCGACGGCGAC GTAAACGGCCACAAGTTCAGCG-3′ (SEQ ID NO: 18), and ssODN-3: 5′-CCTGATTTTATTTCTGTAGG ACTGAACGTCTTGCTTGAGATGTGATGAAGGAGATGGGAGGCCATCACATTG-3′ (SEQ ID NO: 19).

TABLE 1
Bacterial Strains and plasmids

| Strains or plasmids | Description |
|---|---|
| PAK-J | Derivative of a wild type laboratory |
| | P. aeruginosa strain PAK |
| PAK-JΔSTY | PAK-J deleted of exoS, exoT and exoY |
| PAK-JΔpopD | PAK-J deleted of popD |
| PAK-JΔ8 | PAK-JΔSTY deleted of popN, xcpQ, lasI, |
| | rhlI and ndk |
| pUCP19 | Cloning vector for P. aeruginosa |
| pExoS54-FLAG- | pExoS54-Flag fused with TALEN targeting |
| TALEN1 | Venus left DNA-binding site |
| pExoS54-FLAG- | pExoS54-Flag fused with TALEN targeting |
| TALEN2 | Venus right DNA-binding site |
| pExoS54-FLAG- | pExoS54-Flag fused with TALEN targeting |
| HPRT1-T1 | HPRT1 left DNA-binding site |
| pExoS54-FLAG- | pExoS54-Flag fused with TALEN targeting |
| HPRT1-T2 | HPRT1 right DNA-binding site |

Electroporation of P. aeruginosa

1.5 ml of an overnight culture grown in LB medium was harvested in1.5 ml microcentrifuge tubes by centrifugation (1 min, 16,000×g) at room temperature. Each cell pellet was washed twice with 1 ml of room temperature 300 mM sucrose and then resuspended in a total of 100 μl 300 mM sucrose. For electroporation, 100 ng of pExoS54-Flag-TALEN DNA was mixed with 50 μl of electrocompetent cells and transferred into a 2 mm gap width electroporation cuvette (Bio-Rad). After applying a 2.5 kV pulse, 1 ml of LB medium was added immediately, and the cells were transferred to a culture tube (10 ml) and shaken for 1 h at 37° C. Cells were then plated on L-agar plates containing 150 μg Carbenicillin per ml. The plates were incubated at 37° C. until colonies appeared.

### Cell Culture

A GFP-expressing B5 mESC line (EB5) was grown on 0.1% gelatin (Millipore)-coated plates in mESC medium and passaged following dissociation by 0.25% Trypsin/EDTA (Thermo Scientific). A GFP-expressing hESC line (LT2e-H9CAGGFP) and a human iPSC line originated from a male Foreskin (iPS-3) were grown on 5 μg/mL Vitronectin (Life Technologies)-coated plates in mTeSR E8 medium (Life Technologies) and passaged following dissociation by 0.5 mM EDTA (Life Technologies). All cells were cultured at 37° C. with 5% CO₂, and supplemented with Penicillin and Streptomycin (Cellgro). Ciprofloxacin was added to a final concentration of 20 μg/mL to clear protein delivery strain of P. aeruginosa.

To isolate EB5 cell lines harboring expected single-base mutation, GFP-negative cells collected by FACS-Sort were diluted to a final cell density of 5 cells/mL and then plated at 100 μl/well in a 96-well plate coated with 0.1% gelatin. The single clones were checked visually about 6 days after plating and transferred to 24-well coated plates for expended culture, then 6-well plates and finally to 60 mm plates, changing medium every other days. Approximate 4×10⁶ cells were used for genomic DNA extraction following the procedure of the QIAGEN RNA/DNA Mini Kit handbook. A 350 bp long gfp gene fragment was amplified from the genome by PCR and digested with BfaI restriction enzyme to detect single-base mutations.

To select human iPSC clones containing intended single base change in the HPRT1 gene, the cells were cultured for 3 days after TALEN injection, and then subjected to selection in mTeSR E8 medium containing 2.5 μg/ml of 6-thio-guanime (6TG, Sigma) for 6 days. Chromosomal DNA was extracted from the 6TG resistant iPSC cells with the QuickExtract DNA extract solution (Epicentre) and then PCR amplified the 625 bp fragment of the HPRT1 gene. The DNA fragments were cloned into pGEM-T Easy vector (Promega) and randomly chosen clones were subjected to DNA sequencing.

### Plasmid Transfection

Following the optimal transfection condition of FuGENE HD Transfection Reagent (Promega), mouse or human ESCs were seeded in 6-well plates at 70% confluency one day prior to the transfection. TALEN expression plasmid DNA (2 82 g), purified with Qiagen Plasmid Kit, was diluted with cell culture medium to a final volume of 94 μl To this DNA solution, 6 μl FuGENE HD Transfection Reagent was added, then mixed and incubated at room temperature for 15 min. The mixture was added to the cell cultures slowly with gentle mix. Cells were incubated at 37° C. with 5% CO₂ for at least 4 hours before downstream experiments. The same procedure was followed for the introduction of single-stranded oligonucleotide templates into target cells.

### Protein Injection Assay

Mouse or human ESCs were seeded in 6-well plates at approximately 70% confluency in antibiotic-free ES medium. P. aeruginosa strains were grown at 37° C. in LB containing carbenicillin until optical density (OD₆₀₀) reached 1.0. Then the bacterial cells were collected by centrifugation, washed with PBS and diluted in ES medium without antibiotic. The ESCs were co-cultured with bacterial cells at various multiplicity of infection (MOI) for indicated period of time. Infection was terminated by washing ESCs with PBS for three times and culturing on ES medium containing 20 μg/mL of ciprofloxacin.

For Western blot analysis of the injected proteins, cells were collected at indicated time post bacterial infection and centrifuged at 1,700 rpm for 2 min The cell pellets were lysed with 40 μl PBS containing 0.25% Triton-X on ice for 10 min The lysed cells were then centrifuged at 16,000 rpm for 5 min. The nuclear protein extracts were prepared using an extraction kit from Beyotime and followed the manufacturer's instruction. Soluble fraction was collected, mixed with an equal volume of 2× SDS-PAGE loading buffer and boiled for 10 min. Protein samples were separation on 10% SDS-PAGE, transferred onto polyvinylidene fluoride (PVDF) membrane, and then probed with mouse M2 monoclonal antibody against FLAG-tag (Sigma).

### Flow Cytometry

Infected mESCs were collected by 0.25% Trypsin/EDTA treatment for 5 min while infected hESCs were collected by 0.5 mM EDTA treatment for 3 min Cells were centrifuged at 1,700 rpm for 2 min and resuspended in 1 ml ice cold PBS. Cells were analyzed for GFP using Diva v6.2 on LSR-II (BD-Biosciences) and FACS Aria-II (BD-Biosciences) for flow cytometry.

### Results

### Bacterial T3SS Mediated Injection of TALEN Proteins Into Mouse ESC

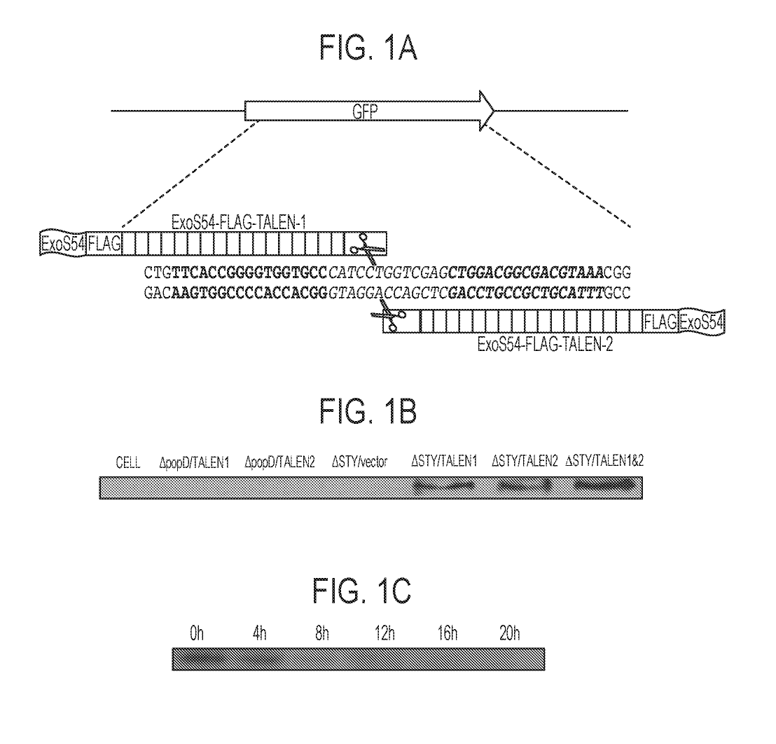

A pair of TALEN constructs, targeting Venus gene, were obtained. This pair of TALENs also targets the gfp gene, encoding Green Fluorescent Protein (GFP), as they share the same target DNA sequence (FIG. 1A). This TALEN pair was delivered into a mouse ESC line (EB5) that stably expresses a gfp gene using the bacterial T3SS delivery system. The two TALENs were each fused with the amino-terminal 54 amino acids of ExoS (ExoS54) which had previously been shown to be an optimal signal sequence for the delivery of exogenous proteins into mammalian cells through the T3SS of P. aeruginosa. The plasmids encoding TALEN fusion proteins, pExoS54-FLAG-TALEN-1 and pExoS54-FLAG-TALEN-2, were each electroporated into two P. aeruginosa strains. Strain PAK-JΔSTY has a high type III secretion capacity and reduced toxicity due to the deletion of endogenous exotoxins. Strain PAK-JApopD is knocked out for a gene encoding a protein required for the formation of translocon pores on the host membrane, and thus incapable of injecting effectors into the host cells. The T3SS-defective strain PAK-JΔpopD was used as a negative control to verify T3SS-dependent injection of the ExoS54-FLAG-TALEN fusion proteins. The EB5 cells were infected with the resulting transformants at a Multiplicity of Infection (MOI) of 100 for 3 hours. The TALEN fusion proteins target EB5 nuclei as they contain nuclear localization sequences (NLS). Following infection by the P. aeruginosa, nuclear proteins of EB5 cells were extracted and subjected to Western blot using anti-FLAG antibody. As the result shown in FIG. 1B, the TALEN fusion proteins were not only efficiently injected into the EB5 cells by P. aeruginosa in a T3SS dependent manner, the injected TALENs are also correctly localized to the nuclei of the EB5 cells.

To determine the intracellular stability of injected TALEN fusion proteins, cells were collected at various time points following the 3-hour infection at MOI of 100. Nuclear proteins were extracted and subjected to Western blot using anti-FLAG antibody. The Western blot result showed that injected proteins were gradually degraded in a time-dependent manner, and became almost undetectable 8 hours after the termination of infection (FIG. 1C).

### Functional Analysis of the Bacterially Injected TALENs.

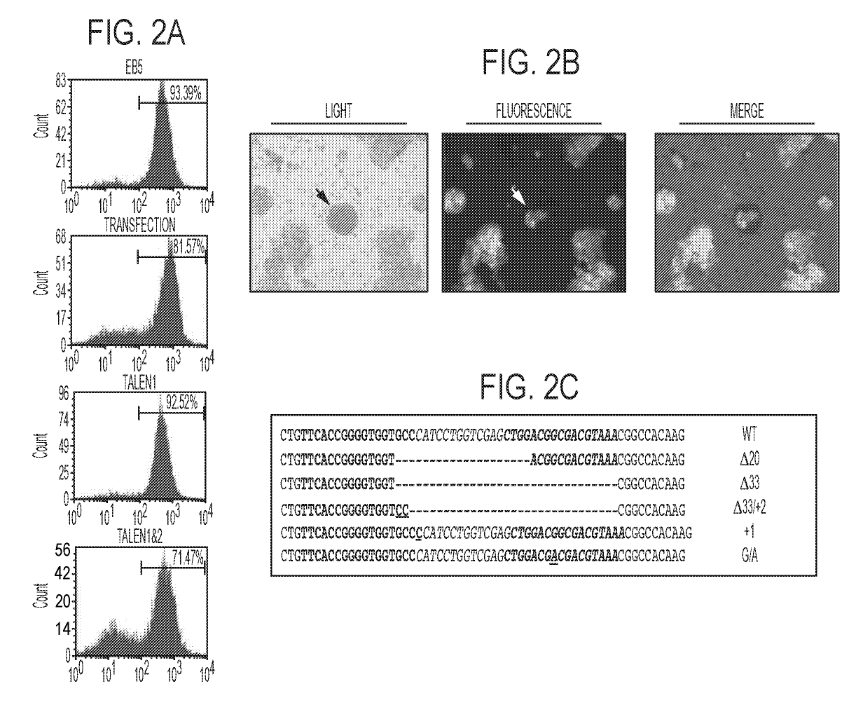

To assess the function of TALEN proteins delivered by the T3SS of P. aeruginosa, GFP fluorescence of the EB5 cells was followed. The EB5 cells were infected by one or two PAK-JΔSTY strains, each harboring one of the pExoS54-FLAG-TALEN pair, at MOI of 100 for 3 hours. The EB5 cells were then washed three times with PBS to remove floating bacterial cells. To remove residual bacterial cells, the EB5 cells were further cultured in mESC medium supplemented by 20 μg/ml Ciprofloxacin. After 3 days of culturing, fluorescence of the EB5 cell population was analyzed by flow cytometry. In parallel, the EB5 cells were transfected with eukaryotic expression plasmids encoding the gfp-targeting TALENs, following the instruction of optimal condition for the transfection reagent, and then cultured in mESC medium for 3 days. According to the FACS analysis results, approximately 20% cells injected of the TALEN protein pair by P. aeruginosa became non-fluorescent, while 10% cells transfected of the plasmid pair became non-fluorescent (FIG. 2A), indicating a two-fold higher gfp-targeting efficiency by the bacterial delivery of TALEN proteins than that of TALEN-coding plasmid transfection. Consistent with the FACS data, three days after the T3SS mediated TALEN pair injection, EB5 cell colonies that lost GFP expression were readily observable under fluorescence microscope (FIG. 2B).

The GFP-negative cells were collected by FACS-Sort and total genomic DNA was extracted. A 350 bp long gfp fragment encompassing the TALEN target site was amplified by PCR and cloned into the TA cloning vector pGEM-T Easy. Sequence analysis of randomly chosen clones identified various mutation types around the TALEN target site (FIG. 2C), presumably resulting from error-prone DNA repair of the DSB generated by the TALEN pair. The T3SS of P. aeruginosa not only effectively delivered the TALENs into mouse ESCs, the injected TALENs also properly executed their biological functions, causing DSB on the target site.

### Bacterial TALEN Delivery Conditions

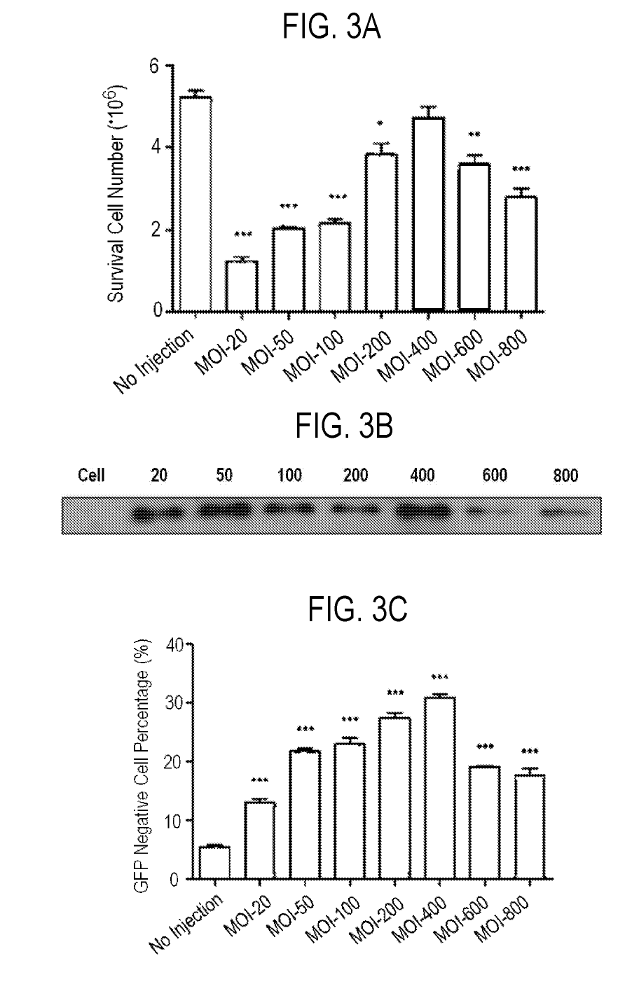

PAK-JΔSTY expressing TALEN to infect EB5 cells for 3 hours at MOIs ranging from 20 to 800. After the infection, floating bacterial cells were removed by washing with PBS, surviving EB5 cells were then counted. Surprisingly, the viability of EB5 cells was the highest at MOI of 400, with lower or higher MOIs resulting in reduced viability (FIG. 3A). The nuclear protein of each sample, derived from the same number of EB5 cells, was extracted and used to detect injected TALEN by Western blot analysis. The Western blot result revealed that the amount of injected TALEN protein increased as the MOI increased from 20 to 400, but decreased beyond MOI of 400 (FIG. 3B). The cells infected at various MOI were cultured for 3 days in mESC medium containing 20 μg/ml ciprofloxacin, and then monitored for GFP fluorescence intensity by flow cytometry. As the results shown in FIG. 3C, about 30% of the cells infected at MOI 400 lost fluorescence, illustrating a further enhancement in the efficiency of TALEN mediated gfp gene knockout.

### TALEN Mediated Single-Base Change on Genomic DNA

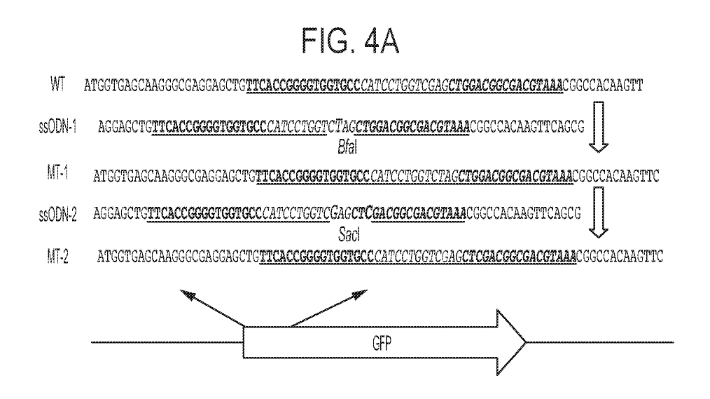

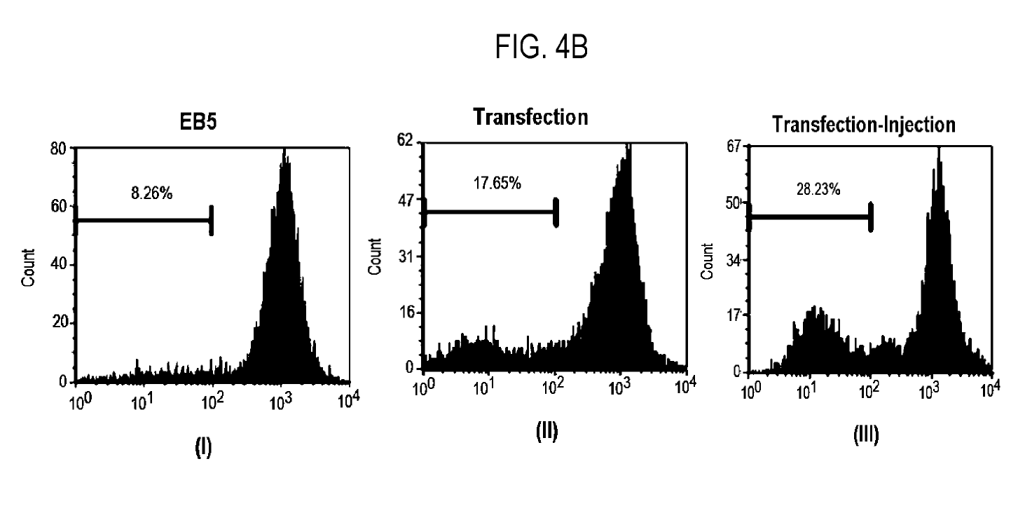

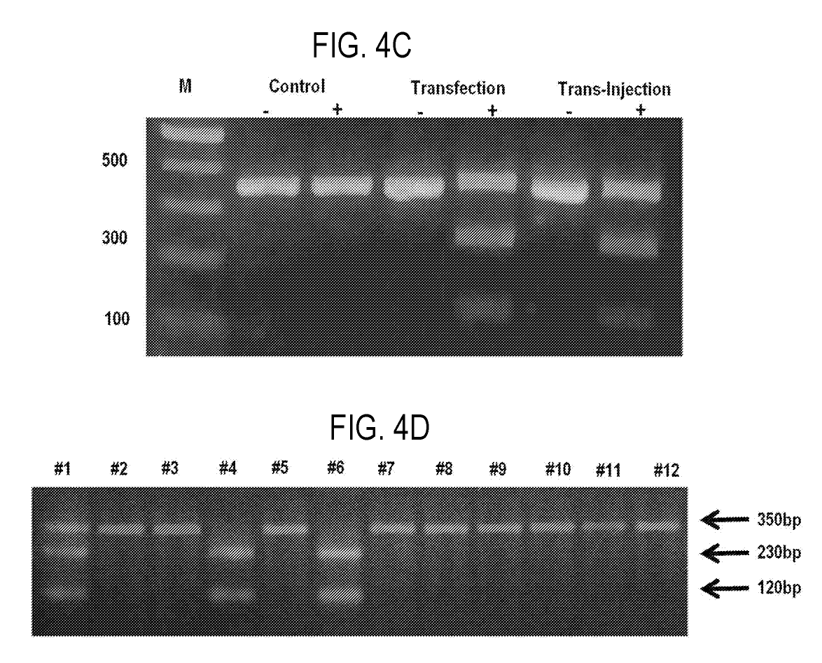

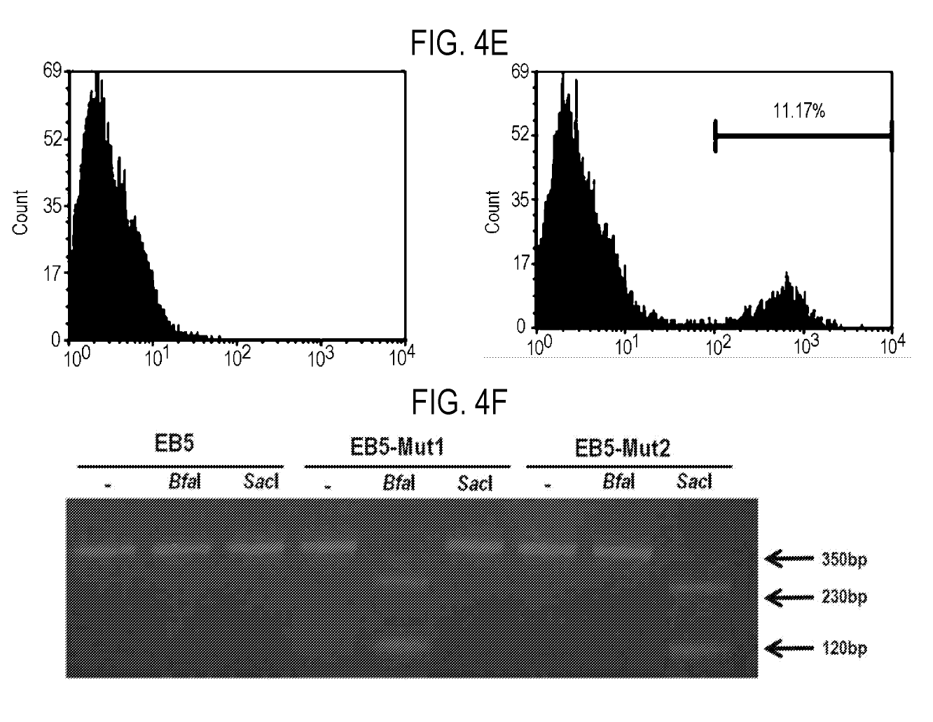

A 72-base long single stranded oligonucleotide DNA (ssODN-1) was designed as a template for homologous recombination in the gfp gene. This ssODN introduces a single nucleotide change, converting a GAG into a stop codon TAG in the GFP open reading frame, which also generates a new BfaI restriction enzyme recognition site (CTAG) (FIG. 4A).

First, EB5 cells were transfected with the ssODN-1. Four hours post-transfection, the EB5 cells were infected with a 1:1 mix of two PAK-JΔSTY strains, each expressing one of the two ExoS54-TALEN fusions, at an overall MOI of 400 for 3 hours. After injection, floating bacterial cells were cleared by washing with PBS and the EB5 cells were cultured in mESC medium containing 20μg/ml ciprofloxacin for 3 days, and then subjected to flow cytometry analysis. As a control, the EB5 cells were transfected with a 1:1 mix of the TALEN pair expressing plasmids together with the ssODN-1 template. Consistent with the gfp gene knockout experiment shown in FIG. 2A, EB5 cells transfected with TALEN-expressing plasmids resulted in about 10% GFP negative cells, while bacterial delivery of the TALEN proteins resulted in almost 20% GFP negative cells (FIG. 4B), indicating that the pre-transfection of template ssODN-1 had no negative effect on the overall gfp gene knockout efficiency. The GFP-negative cells were sorted by FACS in each of the EB5 cell group and their genomic DNA was extracted. The 350 bp TALEN targeting gfp region was amplified by PCR and subjected to digestion by BfaI enzyme. The digestion results showed that both plasmid transfection and TALEN injection by T3SS produced the desired single base change in the genome, resulting in two DNA fragments in sizes of 230 bp and 120 bp (FIG. 4C). Quantitative analysis of the DNA bands by Image-J revealed that approximately 25% of GFP-negative EB5 cells from plasmid transfected and 35% GFP-negative EB5 cells from TALEN injection acquired the new BfaI restriction site. Considering 20% GFP-negative EB5 cells by T3SS mediated TALEN injection, of which 35% had expected single nucleotide change, the overall rate of desired single nucleotide change in the EB5 cell population was 7.0% (20%×35%). On the other hand, in the case of plasmid transfection mediated TALEN delivery, the overall efficiency was 2.5% (10%×25%). Thus, the combination of template ssODN transfection with bacterial injection of the TALEN pair into mESCs resulted in almost 3 folds higher efficiency of target gene modification than the conventional transfection approach.

The EB5 cell line with single-base gfp mutation was further subjected to single cell cloning. The GFP-negative cells obtained by FACS-Sort were diluted and reseeded into a 96-well plate for single cell cloning. Each putative cell clone was expanded through 24-well plate, 6-well plate, and finally to a 60 mm culture plates. About 4×10⁶ cells of each clone were harvested for genomic DNA extraction, and their gfp gene fragment was amplified and subjected to digesting by BfaI restriction enzyme. As the DNA digestion results show (FIG. 4D), two clones (#4 & #6) out of 12 screened had the new BfaI site, while one of them (#1) had a mixture of the two cell types. Sequence analysis of the PCR products of #4 and #6 clones confirmed the presence of correct single-base mutations.

The #4 GFP-negative cell line (EB5-Mut1) containing correct single-base mutation was further reverted back to GFP-positive. A 72-base long ssODN-2 template was designed which introduces 2 single nucleotide changes, one reverting the stop codon TAG back to GAG while the other introduces a new SacI restriction enzyme site (GAGCTC) without changing amino acid sequence (FIG. 4A). The EB5-Mut1 cell line was transfected with the ssODN-2, then infected with a 1:1 mix of the two ExoS54-TALEN delivery strains 4 hours later, at an overall MOI of 400 for 3 hours. After the infection, floating bacterial cells were cleared by washing with PBS and the cells were cultured in mESC medium containing 20 μg/ml ciprofloxacin for 3 days, and then subjected to flow cytometry analysis. According to the FACS analysis results (FIG. 4E), approximately 11% cells reverted back to GFP-positive. The GFP-positive cells were sorted by FACS and their genomic DNA was extracted. A 350 bp fragment of the TALEN targeting gfp region was amplified by PCR and subjected to digestion by Sad enzyme. The 350 bp PCR fragments obtained from EB5 and EB5-Mut1 were used as controls. The digestion results showed that TALEN injection by T3SS produced the desired single base change in the genome, resulting in two DNA fragments in sizes of 230 bp and 120 bp (FIG. 4F). Quantitative analysis of the DNA bands shown in FIG. 4F revealed that almost 100% of GFP-positive EB5 cells (EB5-Mut2) acquired the new Sad restriction site.

T3SS Mediated Injection of TALEN Proteins Into Human ESCs and iPSCs

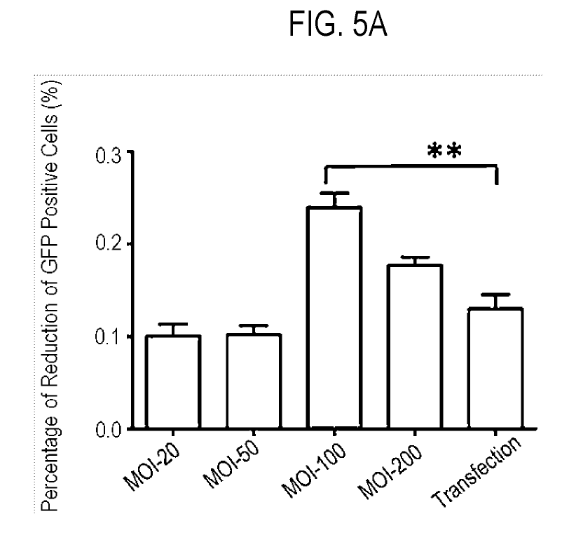

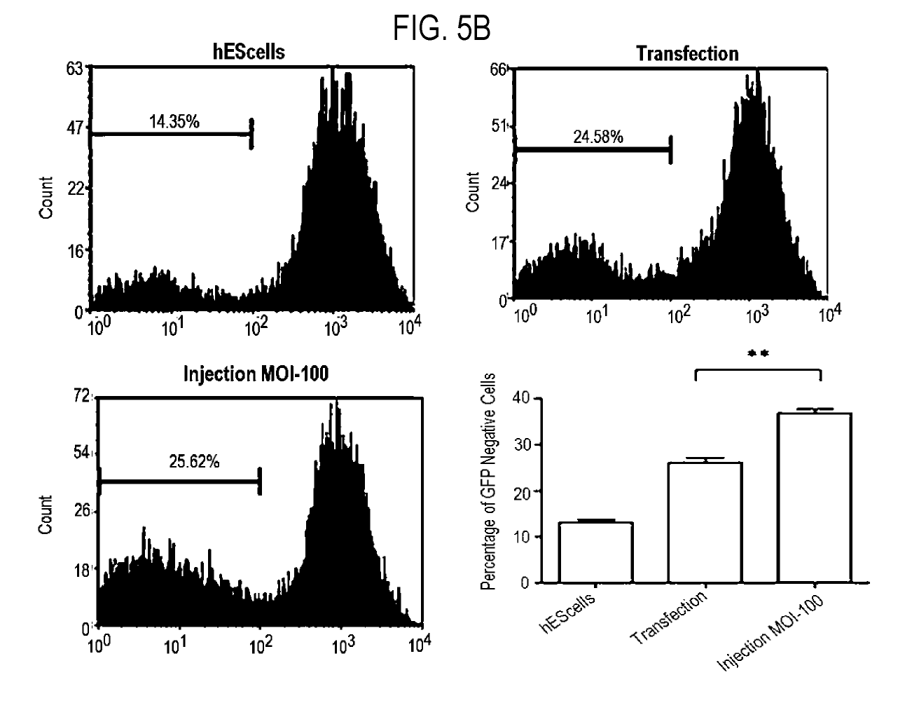

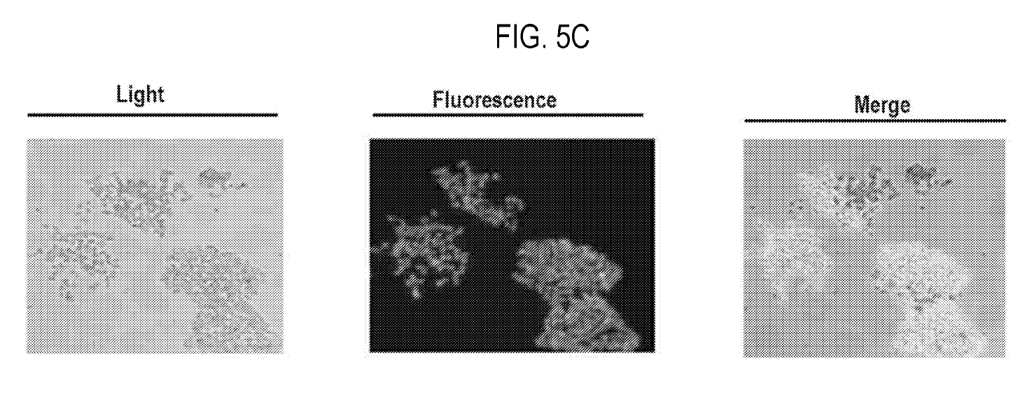

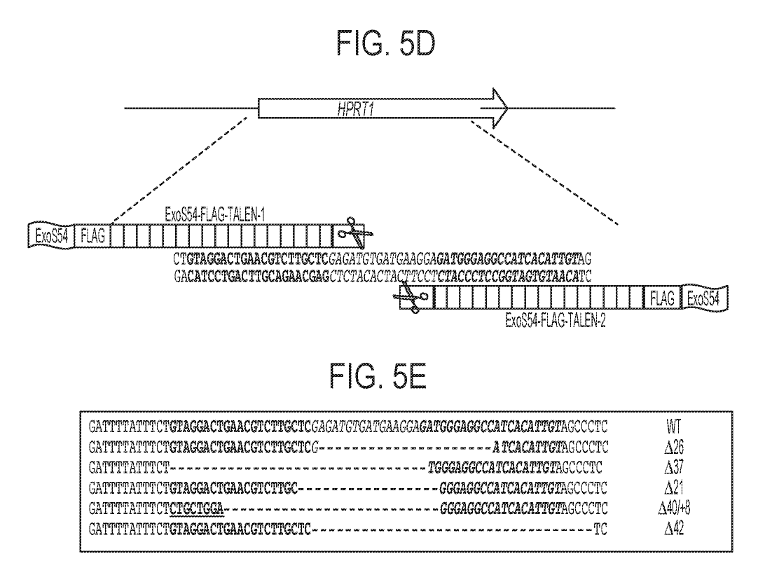

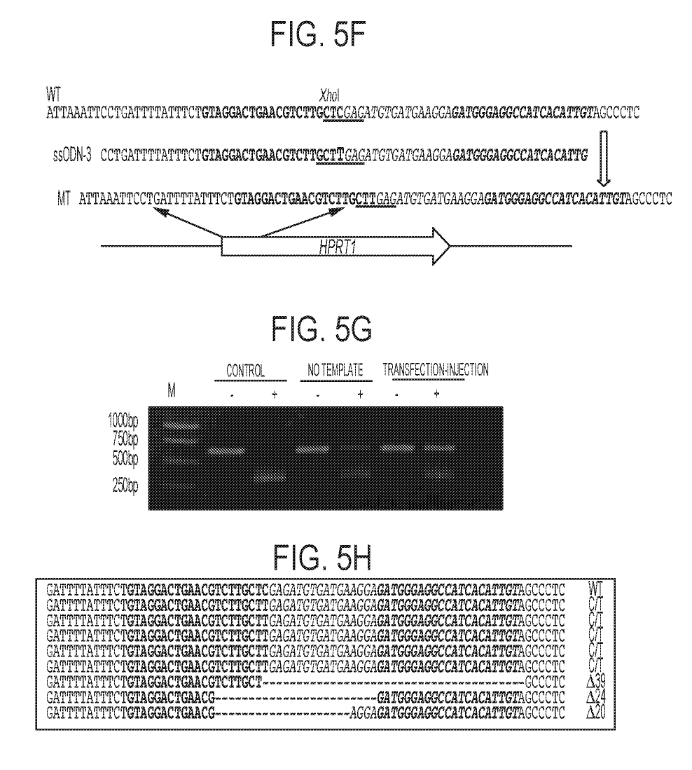

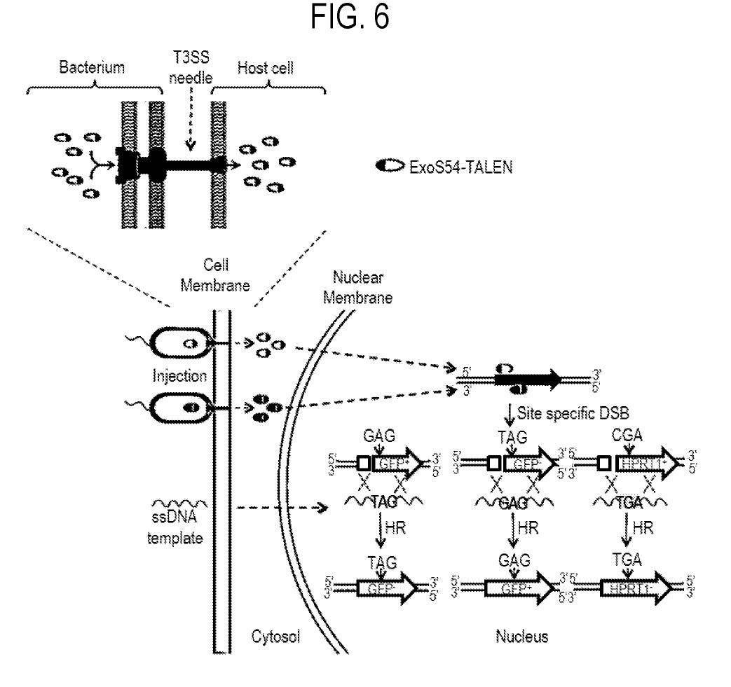

The use of P. aeruginosa strain to inject TALEN proteins into hESCs and hiPSCs was also tested. During initial trials, hESCs and hiPSCs were much more sensitive to the bacterial cytotoxicity than mouse ESCs. To decrease the bacterial cytotoxicity, P. aeruginosa strain PAK-JΔ8 was chosen as the delivery strain. PAK-JΔ8 is deleted of five additional genes from the original delivery strain PAK-JΔSTY, including an inhibitor for the type III secretion (popN), a structural gene for the type II secretion system (xcpQ), genes for quorum sensing synthesis (lasI and rhlI) and a nucleoside diphosphate kinase (ndk) which also displays toxicity against eukaryotic cells. The PAK-JΔ8 shows much lower toxicity than PAK-JΔSTY yet maintains a high type III secretion capacity. hESC line LT2e-H9CAGGFP was seeded at 70% confluency and infected by the two TALEN delivery strains at various MOI for 3 hours. After TALEN injection, the cells were cultured in hESC medium containing 20 μg/ml ciprofloxacin for 3 days, and then monitored GFP fluorescence by flow cytometry. As a control, eukaryotic expression vector plasmids encoding the TALEN pair were delivered by transfection. According to the FACS results, 3 hour infection at MOI of 100 turned out to be optimal for TALEN delivery into the hESC or hiPSC (FIG. 5A). Compared to the control of plasmid transfection, about 10% more GFP-negative cells were obtained by the bacterial delivery under an overall MOI of 100 (FIG. 5B). Non-fluorescent cell clusters of hESCs were detected under fluorescent microscope following bacterial injection of the TALEN pair (FIG. 5C).

A pair of TALEN constructs that target exon 2 of human HPRT1 gene were generated (FIG. 5D). The HPRT1 gene encodes hypoxanthine phosphoribosyltransferase (HPRT) which is responsible for recycling purine. Naturally occurring mutations in the HPRT1 cause decreased levels of the HPRT for purine salvage, leading to neurological and behavioral problems. The HPRT1 gene is located on X chromosome and thus its mutations cause sex-linked diseases. Cells lacking the HPRT activity are resistant to a toxic nucleotide analog 6-thioguanine (6TG) which is metabolized by the HPRT and integrated into the DNA, resulting in cell death, thus cells with a functional HPRT enzyme are poisoned by the 6TG. The HPRT1 targeting TALENs were bacterially injected into a male originated iPSC at 70% confluency under the optimal condition (MOI of 100 for 3 hours). After injection, bacterial cells were washed off with PBS and cells were cultured in the iPSC medium containing 20 μg/ml ciprofloxacin. The cells were cultured for 3 days to allow phenotypic expression prior to drug selection. After 3 days of culture, the cells were selected on iPSC medium containing 2.5 μg/ml of 6TG for 6 days. During the 6TG selection period, most of the uninfected control cells gradually died, while many cells injected of the TALEN proteins by P. aeruginosa T3SS survived and formed visible colonies. Assuming each colony was arisen from a single cell, the overall efficiency of HPRT1 gene mutation was about 1%. The clones were pooled, extracted chromosomal DNA and PCR amplified a 625 bp fragment encompassing the TALEN-targeting region of the HPRT1 gene. The PCR product was cloned into pGEM-T Easy vector and ten clones were randomly chosen for sequence analysis. From the sequencing results, various types of alternations were observed around the TALEN cleavage site (FIG. 5E), indicating that injected TALENs efficiently introduced double stranded DNA breaks, triggering error-prone DNA repair which resulted in the observed HPRT1 gene mutations on the chromosomes of iPSCs.

To generate a desired nucleotide change in the HPRT1 gene of human iPSC, a 72-base long ssODN-3 was designed as a template for homologous recombination. The ssODN-3 introduces a single nucleotide change, converting a CGA into a stop codon TGA in the HPRT1 open reading frame which also destroys an XhoI enzyme digest site (CTCGAG) (FIG. 5F). First, the iPSCs were transfected with the ssODN-3 and 4 hours later, the cells were infected with a 1:1 mix of two PAK-JΔ8 strains, each expressing one of the TALEN pair, at an overall MOI of 100 for 3 hours. After injection, floating bacterial cells were washed off with PBS and the iPSCs were cultured in iPSC medium containing 20 μg/ml ciprofloxacin for 3 days to allow phenotypic expression. The cells were then selected in iPSC medium containing 2.5 μg/ml of 6TG for 6 days and the emerging 6TG-resistant colonies were used for genomic DNA extraction. The 625 bp HPRT1 target sequence was amplified by PCR and the resulting fragment was subjected to digestion by XhoI enzyme. The wild type HPRT1 fragment can be digested by XhoI enzyme into two similar sized DNA fragments (313 bp and 312 bp), while the correct single nucleotide change by homologous recombination (HR) as well as some non-homologous end-joining (NHEJ) lose the XhoI recognition site. The digestion result of “no template” control (FIG. 5G) showed that TALEN injection alone indeed resulted in 20% DNA lost their XhoI site, presumably through mutations during NHEJ. In the experimental sample where both TALEN and ssODN-3 were delivered, about 45% DNA lost their XhoI site (FIG. 5G). The 650 bp HPRT1 fragment insensitive to the XhoI enzyme digestion was gel purified and cloned into the TA cloning vector pGEM-T Easy. Sequence analysis of eight randomly chosen clones identified five with expected single base change and three non-specific deletions around the XhoI site (FIG. 5H). In sum, a combination of template DNA transfection with bacterial injection of TALEN into iPSCs resulted in a high efficiency target gene modification.

### Example 2

### Directed Differentiation of Pluripotent Stem Cells by Bacterial Injection of Defined Transcription Factors

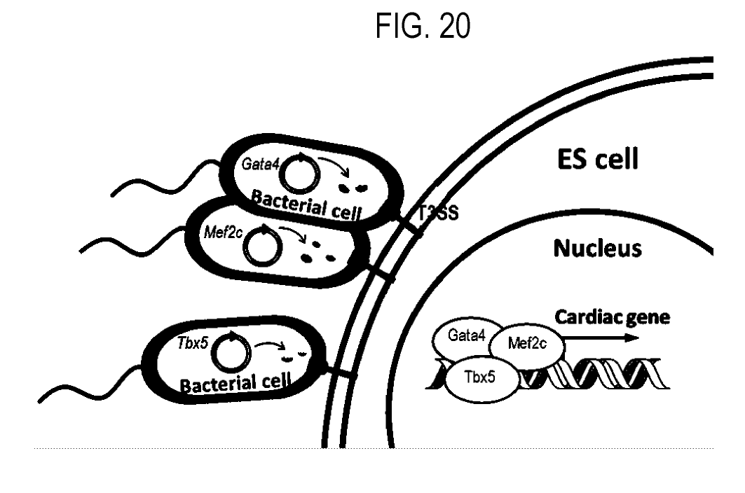

Cardiovascular disease is a leading cause of death worldwide. The limited capability of heart tissue to regenerate highlights the need for developments for creating de novo cardiomyocytes, both in vitro and in vivo. In this example, the T3SS-based protein delivery system was used to direct embryonic stem cell (ESC) differentiation into cardiomyocytes (CMs) by simultaneous injection of multiple transcriptional factors that are relevant to cardiomyocyte development (FIG. 20).

During early heart development, the GMT transcription factors Gata4, Mef2c, and Tbx5 (short as GMT) interact with one another to co-activate cardiac gene expression, such as Actc1 (alpha cardiac actin), cTnT, (cardiac troponin T), and MYH6 (α-myosin heavy chain, also called αMHC), and promote cardiomyocyte differentiation. A bacterial T3SS-based TFs delivery tool to efficiently tanslocate GMT into mouse ESCs is demonstrated. Results indicate that GMT proteins delivered by T3SS are sufficient to activate the expression of cardiac specific genes and promote ESC-CMs differentiation. Further, mesodermal inducer Activin A shows an additive effect on the GMT injection-mediated promotion of ESC-CMs differentiation, allowing higher efficiency of ESC-CMs differentiation than that of spontaneous differentiation. T3SS-based protein delivery system is highly controllable, in terms of injection dose, order and duration.

### Materials and Methods

### Bacterial Strains

The bacterial strains and plasmids used in this example are listed in Table 2. P. aeruginosa were grown in Luria (L) broth or on L agar plates at 37° C. Antibiotics were used at a final concentration of 150 mg carbenicillin per mL.

TABLE 2
Strains and plasmids used in this example

| Strain and plasmid | Description |
|---|---|
| P. aeruginosa | |
| PAK-J | PAK derivative with enhanced T3SS |
| ΔSTY | PAK-J deleted of exoS, exoT, exoY; |
| Δ8 | ΔSTY deleted of ndk, xcpQ, lasR-I, rhlR-I and |
| | popN; |
| ΔexsA | PAK-J deleted of exsA; |
| ΔpopD | PAK-J deleted of popD; |
| Plasmids | |
| pUCP19 | Escherichia-Pseudomonas shuttle vector; Ap^{r} |
| piExoS-Flag | pHW0224, pUCP18 containing catalytically inactive |
| | ExoS with a Flag tag; Cb^{r} |
| pExoS₅₄F | Promoter and N-terminal 54 aa of ExoS fused with |
| | FLAG tag in pUCP19; Cb^{r} |
| pExoS₅₄F-Gata4 | pExoS54F fused with gata4 gene; Cb^{r} |
| pExoS₅₄F-Mef2c | pExoS54F fused with mef2c gene; Cb^{r} |
| pExoS₅₄F-Tbx5 | pExoS54F fused with tbx5 gene; Cb^{r} |

### Cell Culture

HeLa cells were cultured in Dulbecco's Modified Eagle Media (DMEM) supplemented with 10% FBS and 1% penicillin/streptomycin (Gibco). Cells were incubated at 37° C. with 5% CO₂. Murine ES cell lines, R1, CGR8 with an EGFP transgene targeted to the α-cardiac myosin heavy chain promoter (MHC-GFP) and 129/Ola with an EGFP transgene targeted to the Brachyury locus (Brachyury-GFP), were routinely cultured and expanded in ESC medium on 0.1% gelatin (Millipore) coated tissue culture plates. The ESC medium was composed of KnockOut Dulbecco's modified Eagle's medium (DMEM; Gibco) supplemented with 10% knockout serum replacer (SR, Gibco), 1% fetal bovine serum (FBS, Gibco), 25 mM Hepes, 300 μM monothioglycerol (Sigma), penicillin-streptomycin and 1 mM L-glutamine (Gibco), and 10³ units/mL recombinant mouse leukemia inhibitory factor (LIF, Millipore). Ciprofloxacin was added at final concentrations of 20 μg/mL, where noted to clear the protein delivery strain of P. aeruginosa.

### Cytotoxicity Assays

Cells were infected by P. aeruginosa for different hours. After infection, the cells were washed and incubated with 0.25% Trypsin for 5 minutes. The number of cells were then counted under microscope. The lactate dehydrogenase (LDH) release assay used CytoTox96 (Promega) and followed the manufacturer's instruction.

### Protein Production and Secretion Assay

Pseudomonas aeruginosa strains were grown overnight in 2.0 ml of Luria broth containing carbenicillin (150 μg/ml) at 37° C. Overnight cultures were then inoculated at 5% into fresh L broth plus antibiotics, where 5 mM EGTA and 0.2% serum were supplemented for type III inducing condition. P. aeruginosa strains were grown in a shaking incubator at 37° C. for 3-5 h, after which bacterial cells were centrifuged at 20,000 g for 2 min. Bacterial supernatants were collected, precipitated with 15% TCA (20× concentration), resuspended in 1× SDS protein sample buffer and boiled for 15 min before Western Blot analysis.

### Protein Injection Assay

ES cells were seeded at approximately 70% confluence in antibiotic-free medium. P. aeruginosa strains were grown at 37° C. in Luria broth containing carbenicillin until reaching an optical density (OD₆₀₀) of 0.8. ES cells were co-cultured with bacteria at a multiplicity of infection (MOI) of 100 for 3 hours. Infection was terminated by washing cells three times with PBS and growing the cells on ES medium containing 20 μg/mL ciprofloxacin. In the case of immunofluorescence analysis (see below), infections were stopped by fixation with PFA.

For Western Blot analysis, cells were infected as described above Immediately following infection, cells were washed, collected by digestion with 0.25% trypsin, and centrifuged at 500×g for 10 min. The cell pellets were lysed in sodium dodecyl sulfate polyacrylamide gel electrophoresis (SDS-PAGE) loading buffer, and boiled for 15 min.

### Western Blotting

Secretion and injection samples were separated on 4-20% gradient SDS-PAGE gels (Bio-Rad). Proteins were transferred onto PVDF membranes and subjected to immunoblotting using an anti-FLAG antibody (mouse M2 monoclonal Ab; Sigma) for GMT and anti-β-actin (Santa Cruz) for actin, with 1000-fold dilutions.

### Cardiac Differentiation of ES Cells

All murine ESC lines were differentiated. To initiate embryoid body (EB) formation, “hanging drops” composed of 2000 cells in 30 μL of differentiation medium were generated (day-0 of differentiation). The differentiation medium was based on Iscove's modified Dulbecco's medium (IMDM, Gibco) and supplemented with 20% heat inactivated FBS, 0.5 mM monothioglycerol, lacking supplemental LIF. On day-2 of differentiation, the EBs were transferred into gelatin coated 24-well plates with 1-2 EBs per well and cultivated for 2 additional days. From day 5 until day 12, differentiation medium was replaced every 2-3 days. Images of EBs were captured at ×5 magnification with a Leica DMIRB inverted phase contrast fluorescence microscope with a DFC425 camera (Leica) and processed using the Leica Application Suite (LAS) microscope software. The microscopic images of the fluorescent EBs were further analyzed for the quantitative analysis of total fluorescence in each EB. Total fluorescence per EB (TF/EB) was calculated in an excel sheet by applying the measurements obtained from the EBs using Image J software. Total fluorescence per EB (TF/EB)=Integrated Density−(Area of selected EB×Mean fluorescence of background readings).

### Flow Cytometry

For fluorescence-activated cell sorting (FACS) analysis, single cells were dissociated from embryoid bodies on day-12 using TrypLE (Gibco) and fixed by 4% paraformaldehyde (Sigma-Aldrich) in PBS for 30 min at room temperature. Cells were centrifuged at 500×g for 10 minutes and resuspended in 1 ml PBS containing 2% FBS. Cells were analyzed for αMHC-GFP fluorescence using a FACS Calibur (BD-Biosciences) flow cytometer.

### Quantitative Real-Time PCR

Total RNA was isolated from undifferentiated cells (day-0) or from EBs collected on various time points of differentiation protocol with the use of RNeasy mini kit (Qiagen), according to the manufacturer's instructions. Potentially contaminating genomic DNA was digested by DNAse I (Turbo DNA-free, Ambion). The first-strand cDNA was synthesized with High Capacity cDNA Reverse Transcriptase Kit (Applied Biosystems). Real-time PCR reaction was performed using the Power SYBR® Green PCR Master Mix (Applied Biosystems) according to the manufacturer's instructions. Primer sequences are listed in Table 3.

TABLE 3
Primer Sequences for Real Time PCR

| | | |
|---|---|---|
| Mouse Gata4 | Forward | 5′-TCTCACTATGGGCACAGCAG-3′ |
| | | SEQ ID NO: 30 |
| | Reverse | 5′-GGGACAGCTTCAGAGCAGAC-3′ |
| | | SEQ ID NO: 31 |
| Mouse Mef2c | Forward | 5′-ATCCCGATGCAGACGATTCAG-3′ |
| | | SEQ ID NO: 32 |
| | Reverse | 5′-AACAGCACACAATCTTTGCCT-3′ |
| | | SEQ ID NO: 33 |
| Mouse Tbx5 | Forward | 5′-ACTGGCCTTAATCCCAAAACG-3′ |
| | | SEQ ID NO: 34 |
| | Reverse | 5′-ACGGACCATTTGTTATCAGCAA-3′ |
| | | SEQ ID NO: 35 |
| Mouse | Forward | 5′ -TCCCGAGACCCAGTTCATAG-3′ |
| Brachyury | | SEQ ID NO: 36 |
| | Reverse | 5′ -TTCTTTGGCATCAAGGAAGG-3′ |
| | | SEQ ID NO: 37 |
| Mouse dHAND | Forward | 5′-GAGAACCCCTACTTCCACGG-3′ |
| | | SEQ ID NO: 38 |
| | Reverse | 5′-GACAGGGCCATACTGTAGTCG-3′ |
| | | SEQ ID NO: 39 |
| Mouse Nkx2.5 | Forward | 5′-ACATTTTACCCGGGAGCCTA-3′ |
| | | SEQ ID NO: 40 |
| | Reverse | 5′ -GGCTTTGTCCAGCTCCACT-3′ |
| | | SEQ ID NO: 41 |
| Mouse α-MHC | Forward | 5′-CCAGCTAAAGGCTGAGAGGA-3′ |
| | | SEQ ID NO: 42 |
| | Reverse | 5′-AGGCGTAGTCGTATGGGTTG-3′ |
| | | SEQ ID NO: 43 |
| Mouse β-actin | Forward | 5′-TTGCTGACAGGATGCAGAAG-3′ |
| | | SEQ ID NO: 44 |
| | Reverse | 5′-GTACTTGCGCTCAGGAGGAG-3′ |
| | | SEQ ID NO: 45 |

### Immunocytological Staining

For ExoS₅₄-Flag-TFs fusion staining, cells were fixed with 4% formaldehyde in PBS for 15 min at room temperature. Cells were then washed 3× in PBS and permeablized with 0.2% Triton X-100 in PBS. Cells were then washed 3× in 1× PBS with 0.05% Triton X-100 (PBST) and blocked with 1% BSA in PBST for 30 minutes. Cells were incubated with anti-FLAG primary antibody for 2 hours at room temperature, then washed 3× in PBST. Cells were then incubated with secondary antibody for 1 hour at room temperature, washed 3× in PBST, then mounted and stained nucleus with NucBlue® Fixed Cell ReadyProbes® Reagent and examined under fluorescence microscope.

Single cardiomyocytes were isolated from embryoid body (12 d) by trypLE (Gibco) and plated on gelatin-coated glass coverslips. Cells were fixed in 4% paraformaldehyde for 20 min and permeabilized with 0.1% Triton X-100 in 1× PBS for 5-15 min at room temperature. After blocking with 10% goat serum in PBST for 1 h at room temperature, cells were stained with primary antibodies of an anti-sarcomeric α-actinin, diluted 1:100 (Sigma-Aldrich), an anti-cardiac actin, diluted 1:200 (Sigma-Aldrich) and an anti-troponin T, diluted 1:50 (Sigma-Aldrich), for 2 h at room temperature. Cells were rinsed three times with PBST and incubated for 1 h with secondary antibody (Alexa Flour 594-conjugated anti-mouse IgG, 1:200) diluted in PBST containing 10% goat serum. The slides were mounted with Vectashield containing DAPI (Vector Laboratories). Images were visualized under a Leica DMIRB inverted fluorescence microscope, captured with a DFC425 camera (Leica) and processed using the Leica Application Suite (LAS) microscope software.

### Response Surface Methodology

A Box-Behnken design of RSM was employed to optimize the MOI ratio of three factors (Gata4, Mef2c and Tbx5), which were investigated at 3 levels: low level (MOI=10), high level (MOI=50) and the center point (MOI=30), and the experimental design used for this study was shown in Table 4. A total of 15 experiments were conducted by using different MOI ratio of GMT. The corresponding responses, total fluorescence per EB (TF/EB), were calculated by Image J program. Design-Expert, version 7.0 (STAT-EASEinc, Minneapolis, USA), was used for experimental designs and statistical analysis of the experimental data. The analysis of variance (ANOVA) was used to estimate the statistical parameters.

TABLE 4
Box-Behnken Experimental Design of
RSM and the Corresponding Responses

| | Factors (MOI) | | | |
|---|---|---|---|---|
| Run | X₁:Gata4 | X₂:Mef2c | X₃:Tbx5 | Response Y:TF/EB |
| 1 | 10 | 50 | 30 | 49.4 |
| 2 | 10 | 30 | 50 | 32.4 |
| 3 | 50 | 10 | 30 | 87.8 |
| 4 | 10 | 30 | 10 | 56.1 |
| 5 | 30 | 10 | 50 | 47.3 |
| 6 | 30 | 10 | 10 | 72.8 |
| 7 | 30 | 30 | 30 | 75.1 |
| 8 | 50 | 50 | 30 | 54.1 |
| 9 | 30 | 50 | 10 | 68.2 |
| 10 | 50 | 30 | 50 | 56.2 |
| 11 | 30 | 30 | 30 | 77.9 |
| 12 | 30 | 30 | 30 | 80.5 |
| 13 | 10 | 10 | 30 | 58.3 |
| 14 | 50 | 30 | 10 | 64.1 |
| 15 | 30 | 50 | 20 | 34.2 |

MOI: multiplicity of infection; TF/EB: total fluorescence per EB, average of n > 10 Ebs per condition in total.

### Contractile Movement Analysis

On day-12, beating clusters of cells were video recorded using a Leica DMIRB inverted microscope and Leica DFC425 camera with Micro-Manager 1.4 software at an acquisition rate of 50 frames per second (fps) for 10 seconds. After acquisition, videos were converted from TIFF stack to AVI using Image J. The AVI movies were analyzed by a cross-correlation algorithm to track the movement of pixels from frame to frame and to produce effective contractility metrics of the cardiomyocytes. Isoproterenol hydrochloride (ISO), a standard stimulator of the β-adrenergic signaling cascade, and carbachol, a synthetic acetylcholine analogue acting as a cholinergic agonist, were dissolved in serum-free medium and stored according to the manufacturer's guidelines.

### Statistical Analysis

Data were analyzed by the parametric unpaired Student t test. Values with P<0.05 were considered statistically significant.

Development of a Novel Protein Delivery Tool Based on T3SS of P. aeruginosa Suitable for ES Cells.

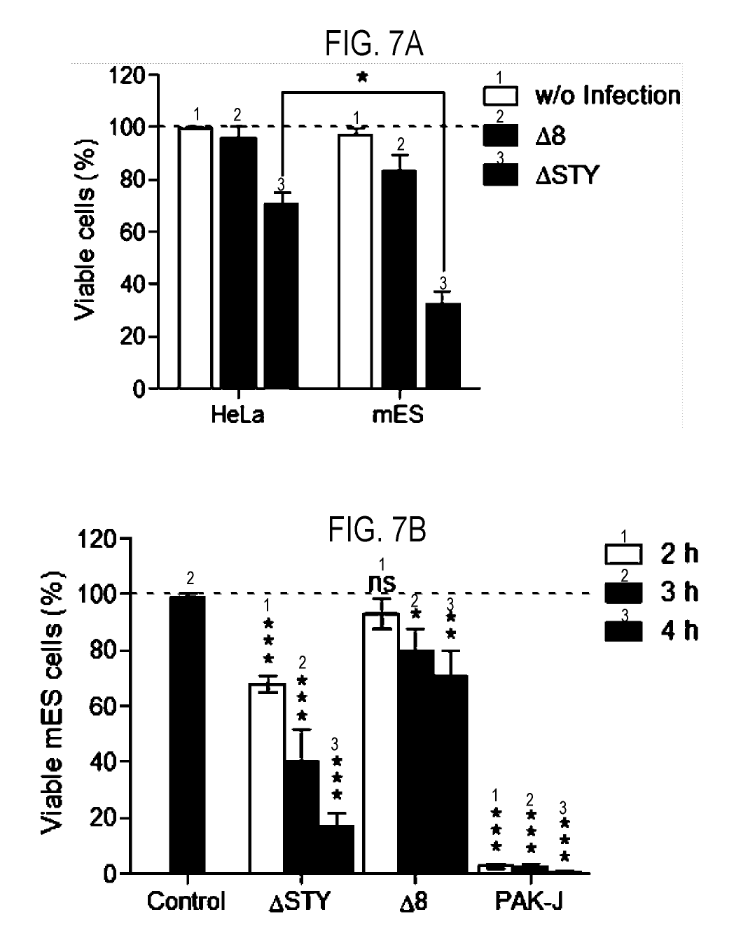

P. aeruginosa strain ΔSTY, which is an engineered low cytotoxicity strain, lacks three well-known endogenous toxin genes (exoS, exoT and exoY) but maintains a high type III secretion capacity (Table 2). However, ASTY shows cytotoxicity on HeLa cells after co-incubation for 3 h at MOI of 100, with approximately 30% of the HeLa cells become rounded and lifted. Pluripotent stem cells (like embryonic stem cells) are much more sensitive to the bacterial cytotoxicity than somatic cells (FIG. 7A). To use P. aeruginosa as a protein delivery vehicle for pluripotent stem cells, the bacterial cytotoxicity needs to be decreased further. To this end, strain ΔSTY was further deleted of genes implicated in the bacterial virulence, including a nucleoside diphosphate kinase gene (ndk), a structural gene for the type II secretion system (xcpQ), genes for quorum sensing signal synthesis (lasI and rhlI), and an inhibitor gene for the type III secretion (popN), resulting in a strain deleted of 8 genes in total, thus designated the resulting strain as Δ8 (Table 2).

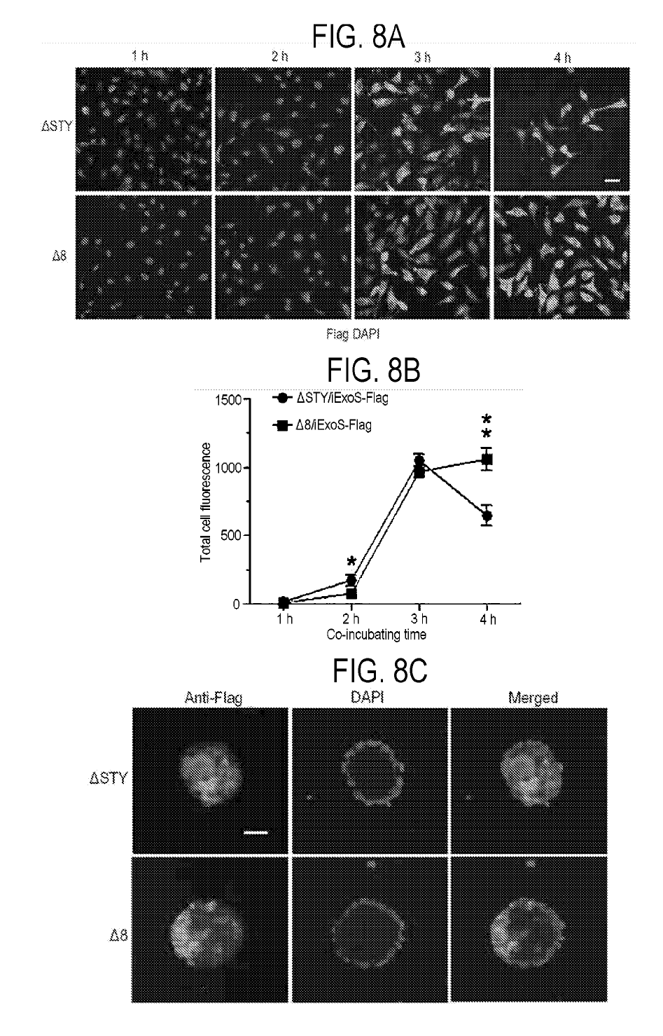

The cytotoxicity of strain Δ8 was compared to that of the wild-type strain PAK-J and ΔSTY. Mouse ES cells were infected by these three strains at MOI 100 for various time and the number of HeLa cells that remain adhered to tissue culture plates were counted. As the results show in FIG. 7B, there was no significant cytotoxicity 3 h post-infection with the Δ8, although by 7 h post-infection, 70% of the cells remain adhered to the plate whereas incubation with the ΔSTY resulted in only 20% of cells still adhering and none with that of wild type PAK-J. Since maximum protein injection is normally achieved by 3 hours of infection, the Δ8 is an appropriate strain for protein delivery. To evaluate the protein injection capability of the Δ8, a fusion of catalytically inactive ExoS with Flag-tag (iExoS-Flag) was injected into HeLa and mES cells by either ΔSTY or Δ8. As shown in FIGS. 8A-8B, the levels of injected iExoS-Flag fusion in HeLa cells by both strains were comparable at MOI=50 for 3 hours. However, about half of the cells were lifted and lysed after infection with ΔSTY for 4 h, while no obvious cell lifting was observed following infection by 48. For mouse ES cells, iExoS-Flag fusion was efficiently injected into the mESCs by Δ8 at MOI of 50 within 3 hours of infection time (FIG. 8C). These results demonstrate that the new Δ8 strain indeed has a much lower cytotoxicity than ΔSTY, yet maintains a high type III secretion capacity.

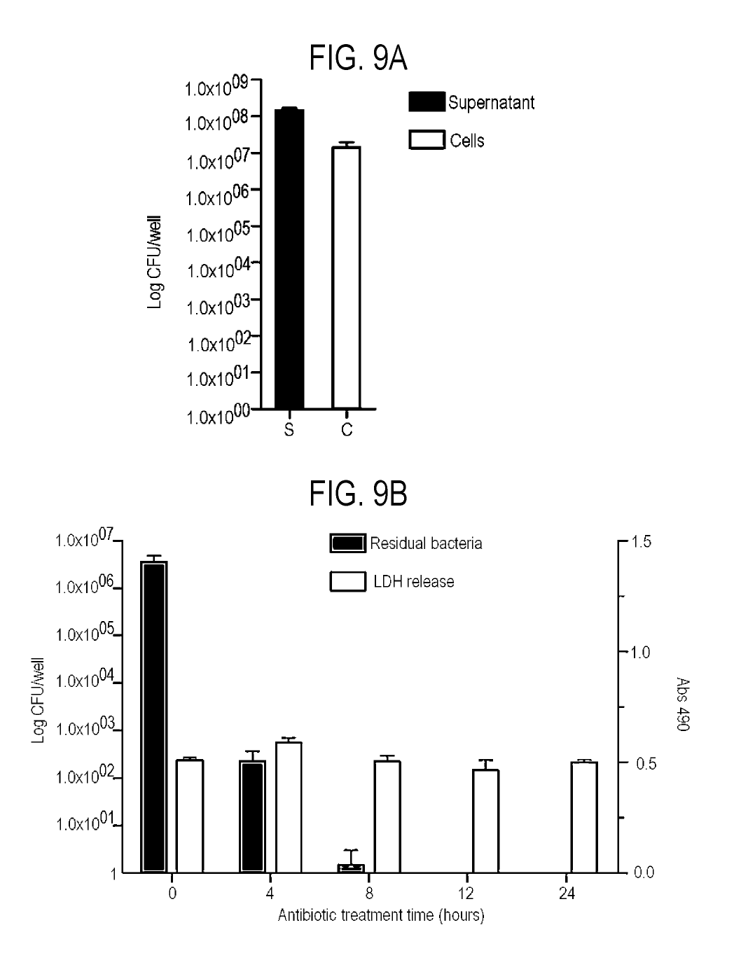

Elimination of the bacterial cells after the completion of protein delivery is another major concern. Following 3 hrs of infection by Δ8 at MOI 100, majority of the bacterial cells (90%) remain floating and can easily be removed by a washing step, but about 10% of input bacterial cells become attached to the ES cells (FIG. 9A). To eliminate the residual adhering bacterial cells, the ES cells were sub-cultured in medium containing 20 μg/mL ciprofloxacin which is an effective antibiotic for P. aeruginosa. ES cells were scraped off from the plate at various time points and viable bacterial cells were enumerated by plating on L-agar medium. As the results show in FIG. 9B, the number of viable bacterial cells gradually deceased, with no detectable bacterial cells by 12 hours. Within the same time frame, treatment of ES cells with the 20 μg/mL ciprofloxacin alone showed no cytotoxic effect.

Bacterial Production and Injection of Transcription Factors into ES Cells.

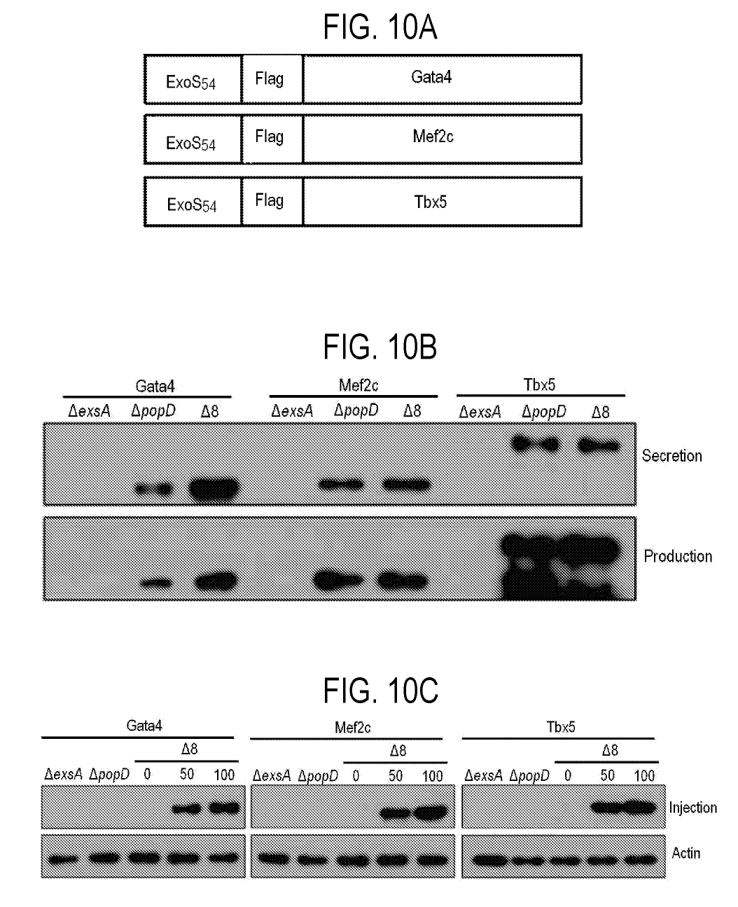

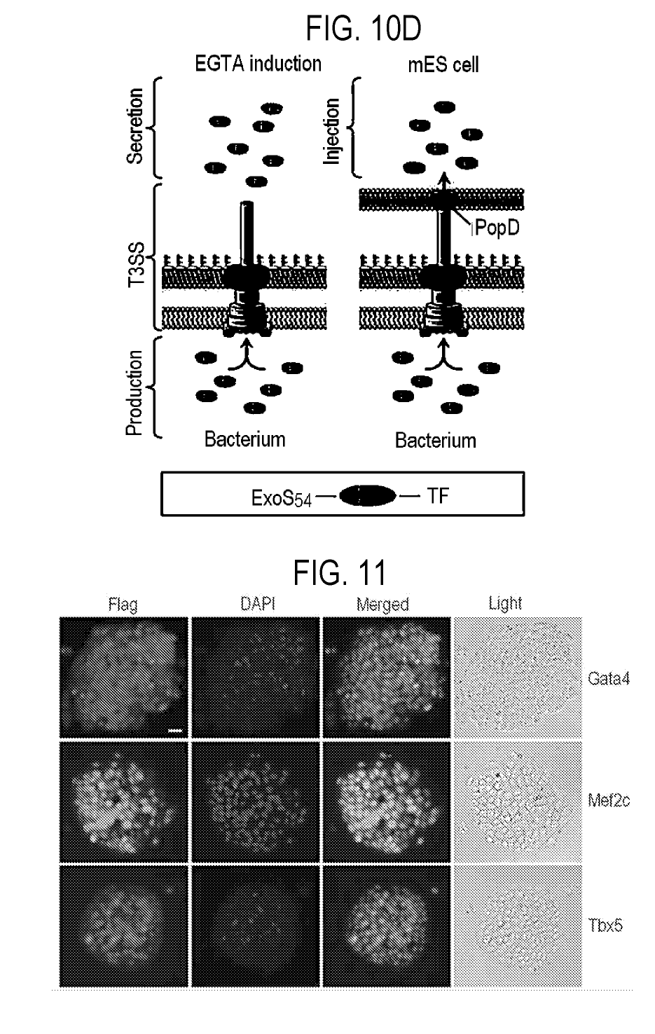

An expression vector pExoS₅₄F was constructed by cloning a DNA fragment containing the P. aeruginosa exotoxin ExoS promoter and N-terminal T3SS secretion signal (ExoS₅₄), followed by a Flag-tag, into the multiple cloning site (MCS) of E. coli-Pseudomonas shuttle vector pUCP19 (Table 2). Three transcriptional factors (TFs), Gata4, Mef2C and Tbx5 were cloned into the pExoS₅₄F, generating in-frame fusions behind the ExoS₅₄-Flag fragment (FIG. 10A). To assess the capacity of P. aeruginosa T3SS to inject transcription factors into ES cells, plasmids pExoS₅₄F-Gata4, pExoS₅₄F-Mef2c and pExoS₅₄F-Tbx5 were each electroporated into three P. aeruginosa strains, ΔexsA, ΔpopD and Δ8, respectively. The resulting transformants were cultured in L-broth in the presence of 5 mM EGTA for 3 hours to induce the type III secretion. Culture supernatants and cells pellets were separated by centrifugation and then subjected to Western blot analysis using anti-Flag antibody. The strain ΔexsA is deleted of a transcriptional activator for the T3SS regulon, thus defective of the type III secretion. Strain ΔpopD contains a functional T3SS that is capable of protein secretion into culture medium, but it is defective in protein injection into the host cells due to the lack of PopD protein required for the formation of the pore on host membrane through which the needle injects effectors. FIG. 10B shows that none of the fusion proteins were expressed or secreted by the T3SS-defective mutant ΔexsA, however, both ΔpopD and Δ8 strains were capable of producing and secreting the fusion proteins, indicating that the ExoS₅₄F-TF fusions could be produced and secreted into culture medium in a T3SS-dependent manner.

To test delivery of the transcription factors into ES Cs, strains of Δ8/Gata4, Δ8/Mef2c or Δ8/Tbx5 were individually co-incubated with mouse ES cells at MOI of 50 for 3 hours. Free floating bacterial cells were subsequently removed by successive washes with PBS, then the ESCs were examined for intracellular fusion proteins by immunoblot or directly immunofluorescence staining. As the results shown in FIG. 10C, none of the fusion factors were injected into ESCs by ΔexsA or ΔpopD, although the fusion proteins were made by the ΔpopD strain. In contrast, all of the fusion proteins could be injected into ESCs by strain Δ8, indicating that the injection of the fusion proteins occurs in a T3SS-dependent manner (FIG. 10D). In addition, the injections occurred in a dose-dependent manner, as there were more translocated fusion proteins when the MOI increased from 50 to 100 (FIG. 10C). All three transcription factors (GMT) could be detected in the nucleus of ESCs (FIG. 11). The injection occurs uniformly on ES cell population, reaching almost 100% target cells at MOI of 50 within 3 hours of infection time. These results demonstrated that the GMTs can be effectively delivered into ES cells by the bacterial T3SS-based protein delivery tool and the translocated proteins are effectively targeted to the nucleus.

### Subcellular Localization and Half-Lives of the T3SS-Injected TFs.

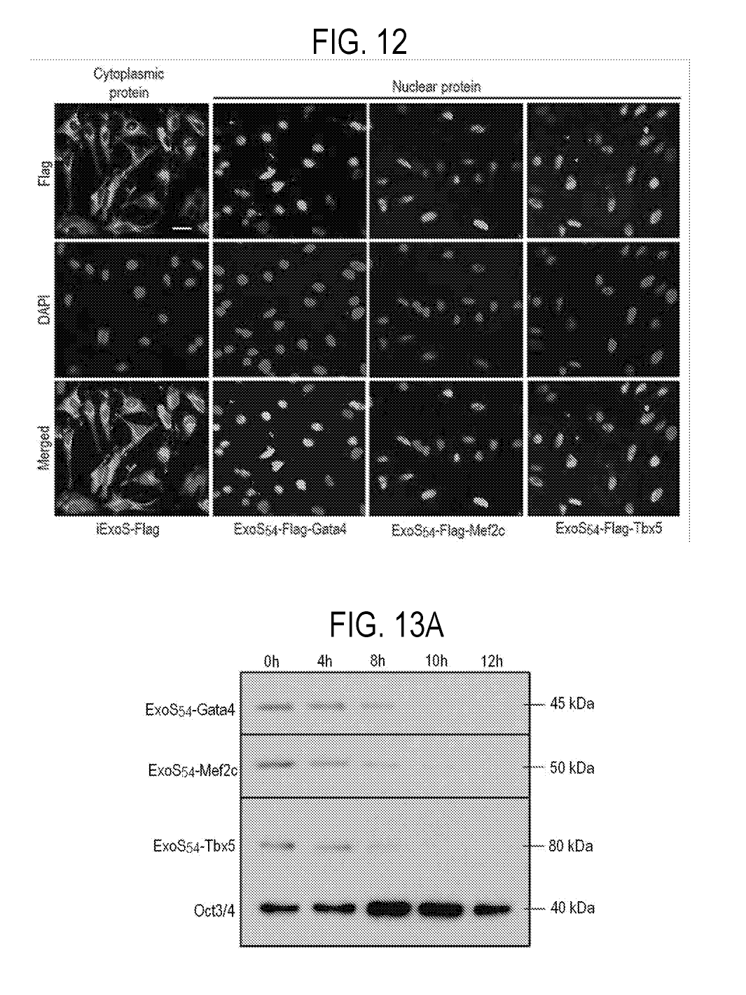

A HeLa cell line was used to study nucleus targeting of injected proteins. Strain Δ8 carrying iExoS-Flag, or the ExoS₅₄-Flag fused with Gata4, Mef2c or Tbx5 were used to infect HeLa cells at MOI of 50 for 4 hours. The intracellular distribution of the translocated proteins was monitored by immunofluorescence staining with an anti-Flag antibody. As FIG. 12 shows, all three ExoS₅₄-TF fusions were predominantly delivered to the nucleus within 4 hours, whereas iExoS-Flag is exclusively found in the cytoplasm, indicating that the N-terminal ExoS ₅₄ -Flag fragment does not interfere with the nuclear localization of the fused transcriptional factors.

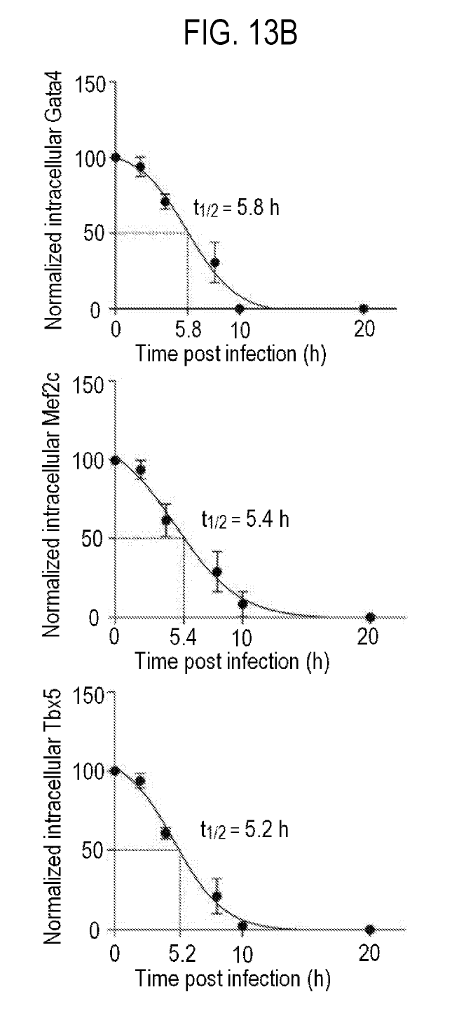

Intracellular proteins are constantly subjected to degradation by proteases at various rates, which was shown dependent on the exposed N-terminal residues in both prokaryotes and eukaryotes. Half-lives of the three ExoS₅₄-TFs fusion proteins within ESCs were determined by Western blot analysis, using the endogenous transcription factor Oct3/4, an undifferentiated ESCs marker, as an internal control. As shown in FIG. 13A, the injected proteins were gradually degraded in a time-dependent manner, till 10 hours post infection. Quantification of the protein band intensities indicated that the half-lives of three ExoS₅₄-TF fusions were all around 5.5 hours (FIG. 13B).

### GMT Delivery Promotes De Novo Differentiation of ES Cells Toward Cardiomyocytes.

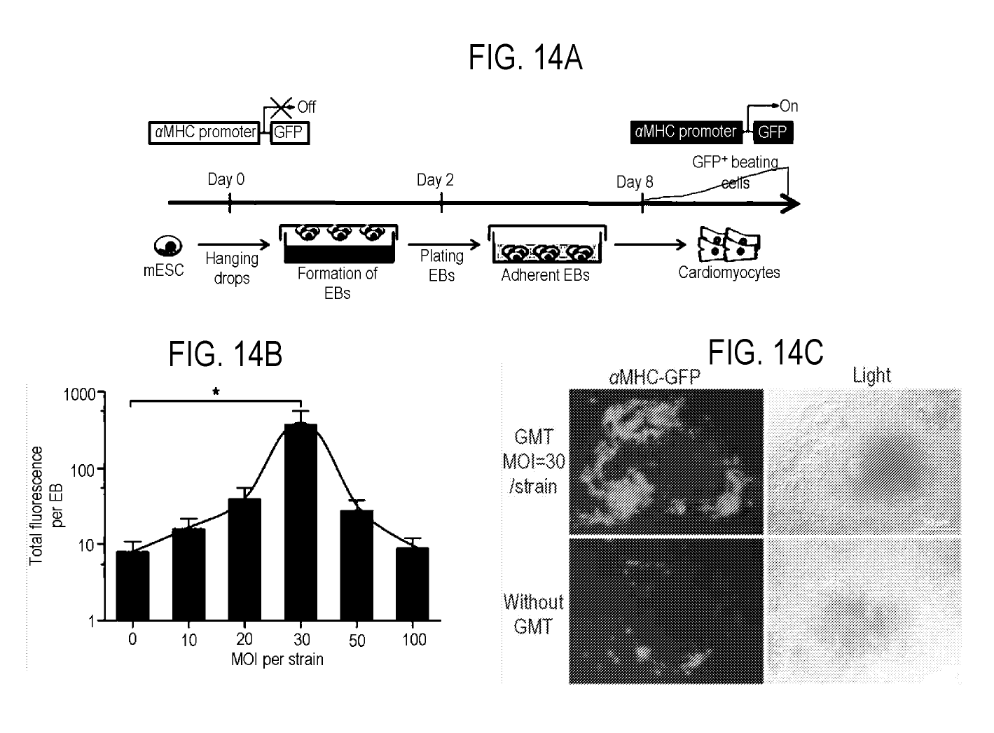

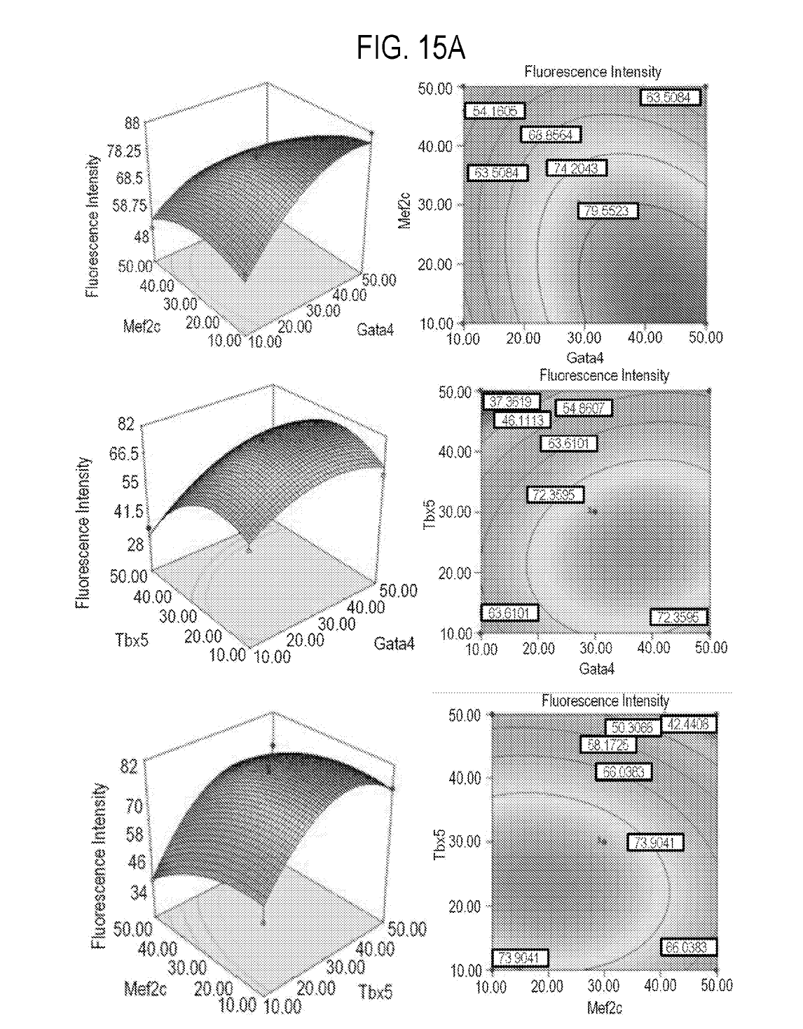

Mouse ES cell line αMHC-GFP, with a GFP transgene driven by α-cardiac myosin heavy chain promoter which is active only in cardiomyocytes, was cultured in hanging drops for 24 hours to form embryoid bodies (EBs). The EBs were transferred into 24-well tissue culture plates on day 2, and allowed for spontaneous differentiation. Starting from day-10, ESC-derived cardiomyocytes (ESC-CMs) can be detected by αMHC-GFP⁺ fluorescence and even spontaneously beating clusters (FIG. 14A). The EBs were subjected to GMT injection individually or in combination at various time points. After 10 days of differentiation, EBs injected of the GMT together showed significant higher GFP fluorescence intensity compared to those injected of the individual factors. Also, a combined delivery of the GMT on day-5 resulted in the highest expression levels of cardiomyocyte marker genes Nkx2.5 and αMHC, indicating the most effective promotion of cardiac program. Bacterial delivery of GMT with a total MOI of 150 did not lead to morphological change of the EBs during differentiation compared to EBs without bacterial infection. To determine the optimal MOI, EBs were infected by each transcription factor delivery strain at MOIs of 10, 20, 30, 50 and 100 on day-5 and the total GFP fluorescence per EB (TF/EB) was recorded on day-12. As the results shown in FIG. 14B, MOI of 30 for each strain, thus the EBs infected by all three delivery strains (GMT) had an overall MOI of 90, showed the highest efficiency of CMs differentiation. Compared to the spontaneously differentiated EBs, more GFP⁺ cardiomyocyte-like cells (αMHC-GFP) appeared in EBs that were injected of GMT combination on day-5 (FIG. 14C). These results demonstrated that GMT combination was able to promote ESCs differentiation into cardiomyocyte-like cells (αMHC-GFP⁺) with a nonlinear (bell curve) dose-dependent manner, indicating that proper ratio and stoichiometry of each transcription factor were necessary for high efficiency differentiation.

### Determination of an Optimal Ratios of the Three Transcriptional Factors for Cardiomyocyte Differentiation.

Response surface methodology (RSM) is a collection of statistical and mathematical techniques used to improve and optimize complex processes. The Box-Behnken design of RSM was chosen to optimize the relative ratio of three factors (Gata4, Mef2c and Tbx5). The experimental design and the corresponding responses were presented in Table 4. The statistical significances of the model and each coefficient were checked by ANOVA analysis and the results are presented in Table 5. The relationship between response (Y) of total fluorescence intensity per EB (TF/EB) and a number of variables denoted by X₁, X₂, X₃, X₁X₂, X₁X₃, X₂X₃, X₁², X₂² and X₃² (X₁, X₂ and X₃ represent MOIs of Gata4, Mef2c and Tbx5, respectively) could be approximated by a second-degree model. ANOVA analysis showed that the second-degree model was significant (P<0.01). Among the variations, only X₁, X₂, X₃, X₁² and X₃² had significant effect on the model, with P-values less than 0.05 (Table 5). Thus, the experimental results could be modeled by a second-order polynomial equation to explain the dependence of total GFP fluorescence intensity of each EB (Y) on the different factors:

Y=12.82+1.99X₁+1.15X₂+1.72X₃−0.024X₁²−0.041X₃²

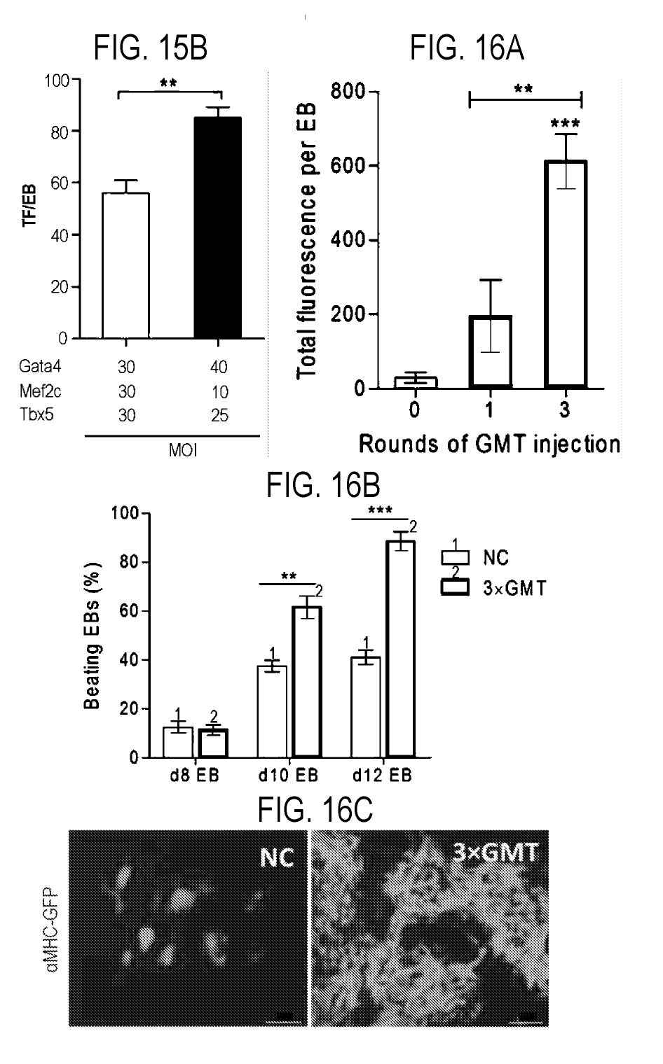

The fit of the model was evaluated by determining coefficient R². The regression equations obtained showed an R² value of 0.9492, indicating that the model could explain 94.92% of the variability in the response. The response surface plots and their respective contour plots for the predicted response Y based on the second-order model are shown in FIG. 15A. They provided prediction of the optimal MOI values for Gata4 (X₁), Mef2c (X₂) and Tbx5 (X₃) to be 40, 10, and 25, respectively. The corresponding experiments were carried out to compare the average Y values between MOI=30:30:30 and MOI=40:10:25 for the three strains (Δ8/Gata4, Δ8/Mef2c and Δ8/Tbx5). The average fluorescence intensity of EBs was indeed significantly higher with the delivery of the three factors at the optimal ratio (FIG. 15B).

TABLE 5
ANOVA for Response Surface Quadratic
Model in Box-Behnken Experiments

| Source of | | | | | | |
|---|---|---|---|---|---|---|
| variation | S.S | D.F | M.S | F value | P value | Signification |
| Model | 3553.87 | 9 | 394.87 | 10.39 | 0.00095 | *Significant |
| X₁-Gata4 | 554.50 | 1 | 544.50 | 14.32 | 0.0128 | |
| X₂-Mef2c | 454.51 | 1 | 454.51 | 11.95 | 0.0181 | |
| X₃-Tbx5 | 1037.40 | 1 | 1037.40 | 27.28 | 0.0034 | |
| X₁X₂ | 153.76 | 1 | 153.76 | 4.04 | 0.1005 | |
| X₁X₃ | 62.41 | 1 | 62.41 | 1.64 | 0.2563 | |
| X₂X₃ | 18.06 | 1 | 18.06 | 0.48 | 0.5213 | |
| X₁² | 328.28 | 1 | 328.28 | 8.63 | 0.0323 | |
| X₂² | 133.11 | 1 | 133.11 | 3.50 | 0.1203 | |
| X₃² | 969.51 | 1 | 969.51 | 25.50 | 0.0039 | |
| Residual | 190.11 | 5 | 38.02 | | | |
| Lack of fit | 175.52 | 3 | 58.51 | 8.02 | 0.1129 | Not |
| | | | | | | significant |
| Pure error | 14.59 | 2 | 7.29 | | | |
| Total | 3743.98 | 14 | | | | |

### Multiple Rounds of GMT Delivery Improve ESC-CM Differentiation.

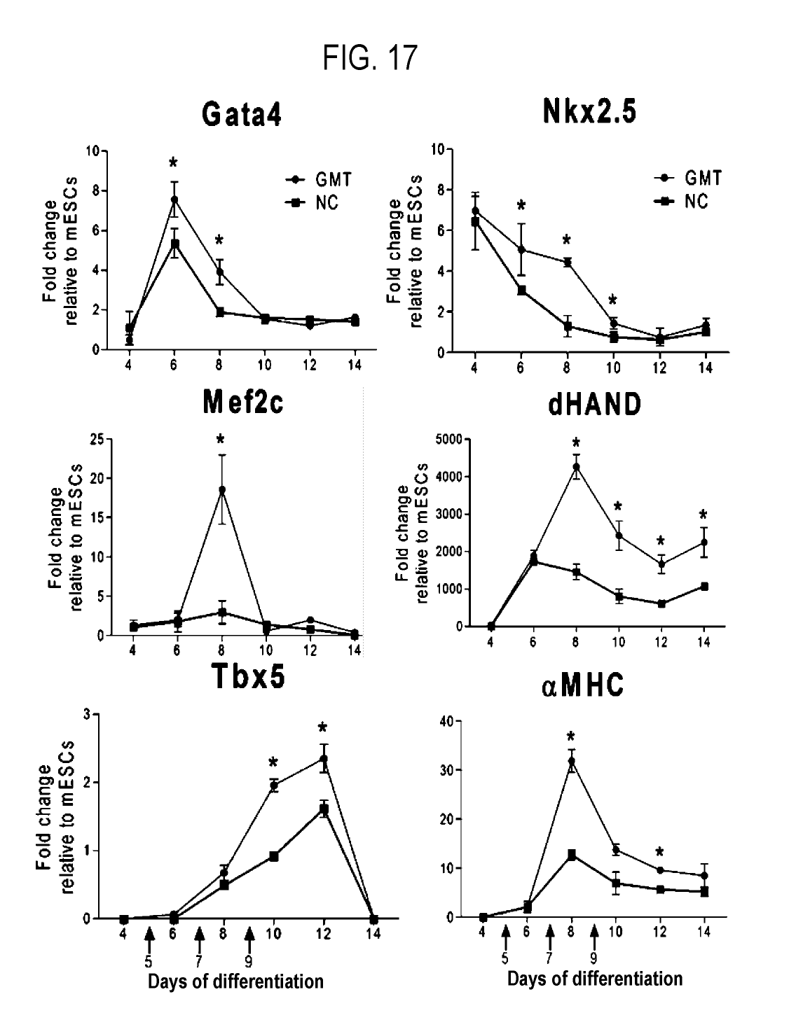

Multiple rounds of GMT delivery enhanced their influence on ESC-CM differentiation. Effects of one time injection of GMT on day-5 was compared to that of multiple rounds of injection at the optimal MOI ratio of the three factors (GMT), evaluating the GFP fluorescence intensity of each EB on day-12. Multiple rounds of GMT delivery (on days 5, 7 and 9) dramatically increased the fluorescence intensity of EBs compared to the one time GMT delivery group, while the latter group was significantly higher than the control group without GMT delivery (FIG. 16A). A continued increase in the number of beating EBs in 3× GMT group was also observed; large contractile areas appeared in ˜85% of the 3× GMT treated EBs on day-12, while only 40% spontaneous differentiated EBs had beating areas that are much smaller in sizes (FIG. 16B). Representative beating clusters composed of GFP⁺ cells are shown in FIG. 16C. Reverse transcription quantitative polymerase chain reaction analysis was performed to further evaluate the effect of exogenous GMT protein delivery on the expression levels of selected cardiac gene. GMT proteins were delivered into EBs at three time points (day-5, 7, and 9) while cardiac gene expression was determined at six time points (day-4, 6, 8, 10, 12, and 14), using EBs without GMT delivery as control. Cardiac transcription factor Gata4, Mef2c, Tbx5, Nkx2.5 and dHand, known as early cardiac progenitor markers, as well as the cardiomyocyte structural gene MYH6 (ΔMHC) were increased dramatically by 3 rounds of GMT delivery (FIG. 17). These results demonstrate that multiple rounds of GMT delivery significantly improve the efficiency of EB differentiation into cardiomyocytes.

### Activin A Shows an Additive Effect on the ESC-CM Differentiation Promoted by the GMT Injection.

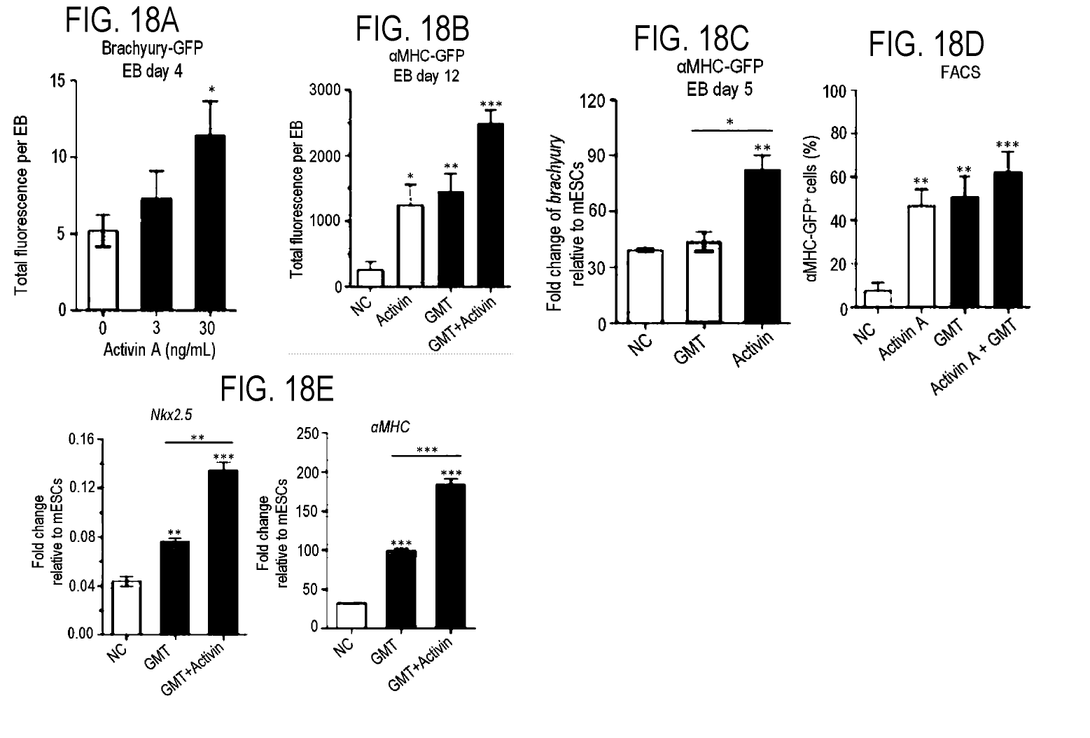

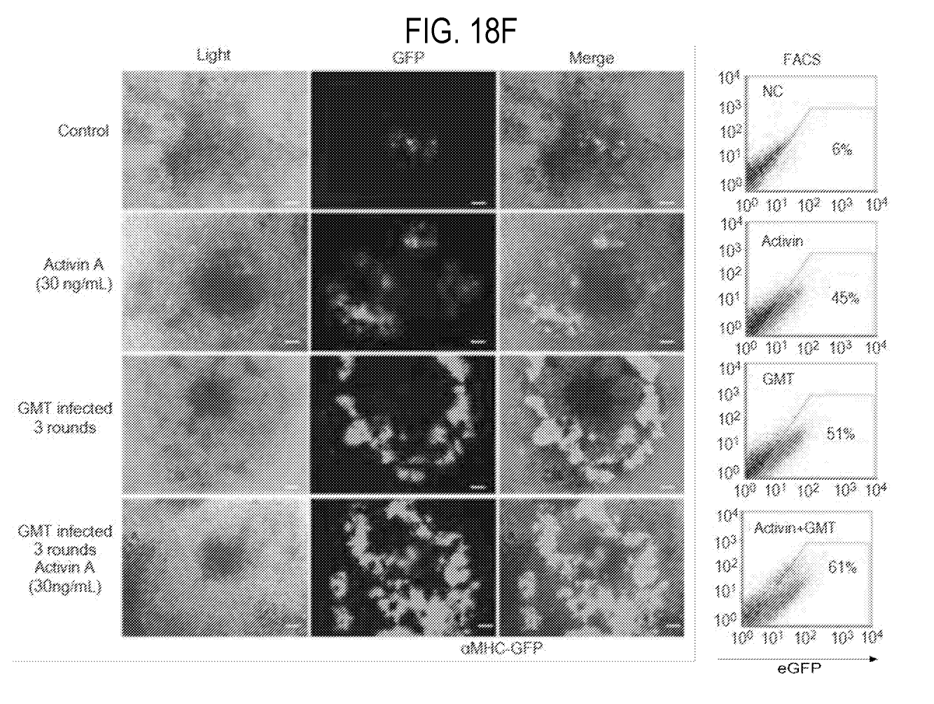

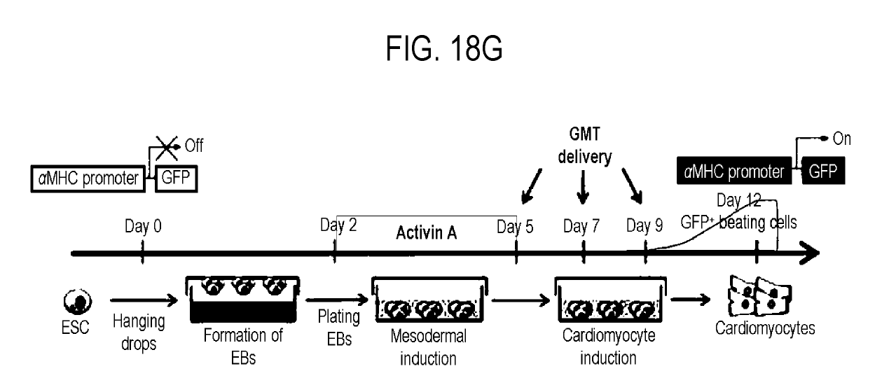

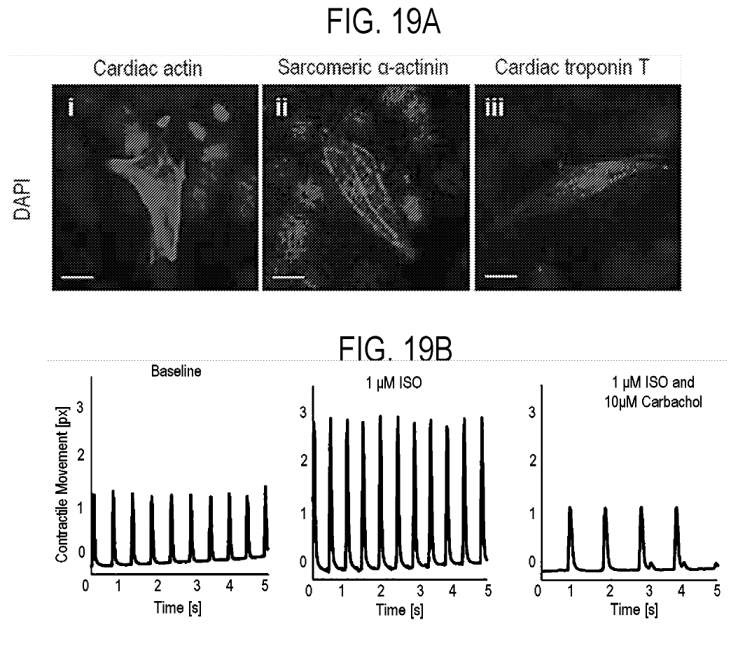

Embryoid bodies (EBs) are three-dimensional aggregates of pluripotent stem cells. ESCs within EBs undergo differentiation and cell specification along the three germ lineages—endoderm, ectoderm, and mesoderm—which comprise all somatic cell types. The cardiac lineages develop from subpopulations of the mesoderm induced in a defined temporal pattern, and expression of Brachyury is commonly used to monitor the onset of mesoderm induction in the ESCs differentiation studies. It had been reported that treatment with proper stoichiometry of Activin A induces mesodermal fate from both mouse and human pluripotent stem cells (PSCs), where high levels of Activin A promote definitive endoderm, moderate levels promote cardiac mesoderm, and low levels promote mesoderm of vascular and hematopoietic lineages. To determine the optimal amount of Activin A required for mesoderm differentiation, EBs were generated using a mouse ESC line with Brachyury-GFP reporter gene and treated with various concentrations of Activin A from day-2 to day-4. As shown in FIG. 18A, Bry-GFP⁺ cells increased in a dose-dependent fashion, with more than two folds increase in GFP fluorescence intensity by day-4 following stimulation with 30 ng/mL of Activin A. For the CMs differentiation from αMHC-GFP ESCs, addition of 30 ng/mL of Activin A from day-2 to day-5 resulted in about 5-fold increase of GFP fluorescence intensity in EBs (FIG. 18B). To further determine whether Activin A could directly induce mesodermal formation, expression of early mesodermal marker Brachyury was determined by q-PCR. On day-5 of EB differentiation, Activin A treated EBs showed higher expression level of the Brachyury, while GMT delivery did not result in such an up-regulation (FIG. 18C), indicating that GMT exert their regulatory effect after the mesodermal stage, while Activin A promotes ESC differentiation towards mesodermal cells.

When Activin A and GMT treatments were combined, the fluorescence intensity of EBs on day-12 increased by about 10 folds compared to those untreated EBs (FIG. 18B). From morphology and fluorescent-assisted cell sorting (FACS) assays (FIG. 18F), about 6% of the αMHC-GFP⁺ cells appeared in the spontaneously differentiated EBs (control), while treating with Activin A for 3 days resulted in 45% of MHC-GFP⁺ cells appeared in or around the center of EBs. Three rounds of GMT deliveries resulted in 51% of MHC-GFP⁺ cells, with the GFP⁺ cells mostly located outside of the EB centers. Most strikingly, combination of the Activin A and GMT deliveries resulted in 61% MHC-GFP⁺ cells in the whole EB cells, representing a 10-fold higher efficiency comparing to the spontaneous differentiation (FIG. 18D), with the GFP⁺ cells appearing both inside and outside of the EB centers (FIG. 18A). In addition, by day-12 of the differentiation, the expression levels of cardiac markers gene Nkx2.5 and α-MHC were significantly higher in the Activin A plus GMT delivery group, compared to either GMT delivery alone or negative control group (FIG. 18E). These results clearly demonstrate an additive effect of the T3SS-mediated GMT delivery and Activin A treatment on the ESC-CMs formation. As summarized in FIG. 18G, 30 ng/mL Activin A treatment from day-2 to day-5, followed by T3SS-mediated GMT delivery at MOIs of 40G:10M:25T for 3 times on days 5, 7 and 9, then assessing the differentiation on day-12 and beyond was successful for generating differentiated cardiomyocytes.

### Characterization of ESC-CMs.

To further evaluate the ESC-CMs, presence of sarcomeric proteins were detected by Immunofluorescence analyses. Cells from 12-day differentiation protocol (Activin A plus GMT delivery) was trypsinized and replated. ESC-CMs were revealed of well-organized cross-striation and positive for cardiac α-actin, sarcomeric α-actinin, and cardiac troponin T (FIG. 19A), demonstrating that ESC-CMs express the cardiac isoform of marker proteins. One of the most critical determinants of normal cardiac physiology is the intact response to hormones and transmitters of the central nervous system. Accordingly, the effects of ISO (1 μmol/L) and carbachol (10 μmol/L) on contractile movement of 12-day old EBs were studied. Videos of ESC-CMs were recorded at 50 fps, and a cross-correlation algorithm was used to detect pixel movement. Average pixel movement over the entire image is plotted versus time. As shown in FIG. 19B, application of ISO led to a typical and comparable increase of the contraction frequency and magnitude of movement (FIG. 19B, middle panel) compared with basal frequency (FIG. 19B, left panel). Subsequent application of carbachol effectively blocked the ISO effect on the beating cells by slowing their contraction frequency as well as magnitude (FIG. 19B, right panel), indicating the presence of intact and coupled β-adrenergic as well as muscarinic signaling cascades.

### Other Embodiments

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present disclosure, and without departing from the spirit and scope thereof, can make various changes and modifications of the disclosure to adapt it to various usages and conditions. Thus, other embodiments are also within the claims.

### Equivalents

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03. It should be appreciated that embodiments described in this document using an open-ended transitional phrase (e.g., “comprising”) are also contemplated, in alternative embodiments, as “consisting of” and “consisting essentially of” the feature described by the open-ended transitional phrase. For example, if the disclosure describes “a composition comprising A and B”, the disclosure also contemplates the alternative embodiments “a composition consisting of A and B” and “a composition consisting essentially of A and B”.

What is claimed is:

1. A Pseudomonas bacterium deficient in exoS, exoT, exoY and popN genes, wherein the bacterium also is deficient for one or more genes selected from the group consisting of: xcpQ, lasR-I, rhlR-I, and/or ndk, said bacterium comprising a polynucleotide encoding a fusion protein, wherein the fusion protein comprises a heterologous protein fused to a bacterial secretion domain.

2. The bacterium of claim 1, wherein the bacterium is a ΔSTYN Pseudomonas bacterium.

3. The bacterium of claim 1 or 2, wherein the bacterium lacks at least one gene selected from the group consisting of lasR-I, rhlR-I, and ndk.

4. The bacterium of any one of claims 1 to 3, wherein the bacterium lacks xcpQ, lasR-I, rhlR-I, and ndk proteins.

5. The bacterium of any one of claims 1 to 4, wherein the heterologous protein is a genome editing protein.

6. The bacterium of claim 5, wherein the genome editing protein is larger than 100 kDa in size.

7. The bacterium of claim 5 or 6, wherein the genome editing protein is a TALEN or a CRISPR/Cas protein.

8. The bacterium of any one of claims 1 to 7, wherein the polynucleotide is on a plasmid.

9. The bacterium of any one of claims 1 to 8, wherein the Pseudomonas is P. aeruginosa, P. alcaligenes, P. anguilliseptica, P. citronellolis, P. flavescens, P. jinjuensis, P. mendocina, P. nitroreducens, P. oleovorans, P. pseudoalcaligenes, P. resinovorans, or P. straminae.

10. The bacterium of any one of claims 1 to 9, wherein the Pseudomonas is P. aeruginosa.

11. The bacterium of claim 10, wherein the P. aeruginosa is PAK-J.

12. The bacterium of any one of claims 1 to 11, wherein the bacterial secretion domain is ExoS17, ExoS54, ExoS96, or ExoS234.

13. The bacterium of any one of claims 1 to 12, wherein the bacterial secretion domain is ExoS54.

14. The bacterium of claim 4, wherein the bacterium exhibits reduced cytotoxicity to human stem cells compared to cells that do not lack xcpQ, lasR-I, rhlR-I, and ndk proteins.

15. The bacterium of claim 14, wherein the human stem cells are embryonic stem cells (hESCs) and/or induced pluripotent stem cells (hiPSCs).

16. A method of delivering one or more proteins into one or more isolated cells, comprising:
incubating the cell or cells with a Pseudomonas bacterium deficient in exoS, exoT, exoY and popN genes, wherein the bacterium also is deficient for one or more genes selected from the group consisting of xcpQ, lasR-I, rhlR-I, and/or ndk, said bacterium comprising a polynucleotide encoding a fusion protein, wherein the fusion protein comprises a heterologous protein fused to a bacterial secretion domain; and incubating the isolated cell or cells for a period of time sufficient to deliver the one or more proteins into said cell or cells.

17. The method of claim 16, wherein the bacterium is a ΔSTYN Pseudomonas bacterium.

18. The method of claim 16 or 17, wherein the bacterium lacks at least one gene selected from the group consisting of lasR-I, rhlR-I, and ndk.

19. The method of any one of claims 16-18, wherein the bacterium lacks xcpQ, lasR-I, rhlR-I, and ndk proteins.

20. The method of any one of claims 16 to 19, wherein the heterologous protein is a genome editing protein.

21. The method of claim 20, wherein the genome editing protein is larger than 100 kDa in size.

22. The method of claim 21, wherein the genome editing protein is a TALEN or a CRISPR/Cas protein.

23. The method of any one of claims 16 to 22, wherein the polynucleotide is on a plasmid.

24. The method of any one of claims 16 to 23, wherein the Pseudomonas is P. aeruginosa, P. alcaligenes, P. anguilliseptica, P. citronellolis, P. flavescens, P. jinjuensis, P. mendocina, P. nitroreducens, P. oleovorans, P. pseudoalcaligenes, P. resinovorans, or P. straminae.

25. The method of any one of claims 16 to 24, wherein the Pseudomonas is P. aeruginosa.

26. The method of claim 25, wherein the P. aeruginosa is PAK-J.

27. The method of any one of claims 16 to 26, wherein the bacterial secretion domain is ExoS17, ExoS54, ExoS96, or ExoS234.

28. The method of claim 27, wherein the bacterial secretion domain is derived from ExoS54.

29. The method of any one of claims 16 to 28, wherein the one or more isolated cells are stem cells.

30. The method of claim 29, wherein the stem cells are human stem cells.

31. The method of claim 30, wherein the human stem cells are embryonic stem cells (hESCs) and/or induced pluripotent stem cells (hiPSCs).

32. The method of claim 19, wherein the bacterium exhibits lower cytotoxicity to human stem cells compared to cells that do not lack xcpQ, lasR-I, rhlR-I, and ndk proteins.

33. The method of any one of claims 16 to 32, further comprising transfecting the one or more isolated cells with a single-stranded oligonucleotide DNA (ssODN).

34. The bacterium of any one of claims 1 to 3, wherein the heterologous protein is a transcription factor.

35. The bacterium of claim 34, wherein the transcription factor is selected from the group consisting of Gata4, Mef2c, and Tbx5.

36. The bacterium of claim 34 or 35, wherein the polynucleotide is on a plasmid.

37. The bacterium of any one of claims 34 to 36 wherein the Pseudomonas is P. aeruginosa, P. alcaligenes, P. anguilliseptica, P. citronellolis, P. flavescens, P. jinjuensis, P. mendocina, P. nitroreducens, P. oleovorans, P. pseudoalcaligenes, P. resinovorans, or P. straminae.

38. The bacterium of any one of claims 34 to 37, wherein the Pseudomonas is P. aeruginosa.

39. The bacterium of claim 38, wherein the P. aeruginosa is PAK-J.

40. The bacterium of any one of claims 34 to 39, wherein the bacterial secretion domain is ExoS17, ExoS54, ExoS96, or ExoS234.

41. The bacterium of any one of claims 34 to 40, wherein the bacterial secretion domain is ExoS54.

42. The bacterium of claim 34, wherein the bacterium exhibits reduced cytotoxicity to human stem cells compared to cells that do not lack xcpQ, lasR-I, rhlR-I, and ndk proteins.

43. The bacterium of claim 42, wherein the human stem cells are embryonic stem cells (hESCs) and/or induced pluripotent stem cells (hiPSCs).

44. The method of any one of claims 16 to 19, wherein the heterologous protein is a transcription factor.

45. The method of claim 44, wherein the transcription factor is selected from the group consisting of Gata4, Mef2c, and Tbx5.

46. The method of claim 44 or 45, wherein the polynucleotide is on a plasmid.

47. The method of any one of claims 44 to 46, wherein the Pseudomonas is P. aeruginosa, P. alcaligenes, P. anguilliseptica, P. citronellolis, P. flavescens, P. jinjuensis, P. mendocina, P. nitroreducens, P. oleovorans, P. pseudoalcaligenes, P. resinovorans, or P. straminae.

48. The method of any one of claims 44 to 47, wherein the Pseudomonas is P. aeruginosa.

49. The method of claim 48, wherein the P. aeruginosa is PAK-J.

50. The method of any one of claims 44 to 49, wherein the bacterial secretion domain is ExoS17, ExoS54. ExoS96, or ExoS234.

51. The method of claim 50, wherein the bacterial secretion domain is derived from ExoS54.

52. The method of any one of claims 44 to 51, wherein the one or more isolated cells are stem cells.

53. The method of claim 52, wherein the stem cells are human stem cells.

54. The method of claim 53, wherein the human stem cells are embryonic stem cells (hESCs) and/or induced pluripotent stem cells (hiPSCs).

55. The method of claim 44, wherein the bacterium exhibits lower cytotoxicity to human stem cells compared to cells that do not lack xcpQ, lasR-I, rhlR-I, and ndk proteins.

56. A method for inducing differentiation of a cell or cells to a cardiomyocyte, the method comprising:
(a) incubating the cell or cells with a first bacterium as described in any one of claims 34 to 43;
(b) incubating the cell or cells with a second bacterium as described in any one of claims 34 to 43; and,
(c) incubating the cell or cells with a third bacterium as described in any one of claims 34 to 43,
wherein the first bacterium encodes Gata4, the second bacterium encodes Mef2c, and the third bacterium encodes Tbx5.

57. The method of claim 56, further comprising washing the cell or the cells to remove the bacteria.

58. The method of claim 56 or 57, further comprising incubating the cell or cells with (a), (b), and (c) a second time.

59. The method of claim 58, further comprising washing the cell or the cells to remove the bacteria, and incubating the cell or cells with (a), (b), and (c) a third time.

60. The method of any one of claims 56 to 59 further comprising incubating the cell or cells with a growth factor.

61. The method of claim 60, wherein the growth factor is Activin A.

62. The method of any one of claims 56 to 61, wherein the relative multiplicity of infection (MOI) ratio of the first bacterium to the second bacterium to the third bacterium ranges from 1:1:1 to 4:1:2.5.

63. The method of any one of claims 56 to 62, wherein the Gata, the Mef2c and/or the Tbx5 is expressed by the cell or cells and has an intracellular half-life of between about 4 and about 6 hours.

64. The method of any one of claims 56 to 63, wherein incubating the cell or cells with at least one of (a), (b) and (c) results in expression of sarcomeric α-actinin, cardiac actin and/or troponin by the cell or cells.

65. The method of any one of claims 56 to 64, wherein the cell or cells are selected from the group consisting of: stem cell(s) and fibroblast(s).

66. A cardiomyocyte or cardiomyocytes produced by the method of any one of claims 56 to 65.


SEQUENCE LISTING (70 sequences)

SEQ ID NO: 1 - protein, 453 aa, Pseudomonas
MHIQSLQQSPSFAVELHQAASGRLGQIEARQVATPSEAQQLAQRQDAPKGEGLLARLGAALVRPFVAIMDWLGKLLGSHARTGPQPSQDAQPAVMSSAVVFKQMVLQQALPMTLKGLDKASELATLTPEGLAREHSRLASGDGALRSLSTALAGIRAGSQVEESRIQAGRLLERSIGGIALQQWGTTGGAASQLVLDASPELRREITDQLHQVMSEVALLRQAVESEVSRVSADKALADGLVKRFGADAEKYLGRQPGGIHSDAEVMALGLYTGIHYADLNRALRQGQELDAGQKLIDQGMSAAFEKSGQAEQVVKTFRGTRGGDAFNAVEEGKVGHDDGYLSTSLNPGVARSFGQGTISTVFGRSGIDVSGISNYKNEKEILYNKETDMRVLLSASDEQGVTRRVLEEAALGEQSGHSQGLLDALDLASKPERSGEVQEQDVRLRMRGLDLA

SEQ ID NO: 2 - protein, 457 aa, Pseudomonas
MHIQSSQQNPSFVAELSQAVAGRLGQVEARQVATPREAQQLAQRQEAPKGEGLLSRLGAALARPFVAIIEWLGKLLGSRAHAATQAPLSRQDAPPAASLSAAEIKQMMLQKALPLTLGGLGKASELATLTAERLAKDHTRLASGDGALRSLATALVGIRDGSRIEASRTQAARLLEQSVGGIALQQWGTAGGAASQHVLSASPEQLREIAVQLHAVMDKVALLRHAVESEVKGEPVDKALADGLVEHFGLEAEQYLGEHPDGPYSDAEVMALGLYTNGEYQHLNRSLRQGRELDAGQALIDRGMSAAFEKSGPAEQVVKTFRGTQGRDAFEAVKEGQVGHDAGYLSTSRDPGVARSFAGQGTITTLFGRSGIDVSEISIEGDEQEILYDKGTDMRVLLSAKDGQGVTRRVLEEATLGERSGHGEGLLDALDLATGTDRSGKPQEQDLRLRMRGLDLA

SEQ ID NO: 3 - protein, 378 aa, Pseudomonas
MRIDGHRQVVSNATAQPGPLLRPADMQARALQDLFDAQGVGVPVEHALRMQAVARQTNTVFGIRPVERIVTTLIEEGFPTKGFSVKGKSSNWGPQAGFICVDQHLSKREDRDTAEIRKLNLAVAKGMDGGAYTQTDLRISRQRLAELVRNFGLVADGVGPVRLLTAQGPSGKRYEFEARQEPDGLYRISRLGRSEAVQVLASPACGLAMTADYDLFLVAPSIEAHGSGGLDARRNTAVRYTPLGAKDPLSEDGFYGREDMARGNITPRTRQLVDALNDCLGRGEHREMFHHSDDAGNPGSHMGDNFPATFYLPRAMEHRVGEESVRFDEVCVVADRKSFSLLVECIKGNGYHFTAHPDWNVPLRPSFQEALDFFQRKV

SEQ ID NO: 4 - protein, 687 aa, Pseudomonas
MHIQSLGATASSLNQEPVETPSQAAHKSASLRQEPSGQGLGVALKSTPGILSGKLPESVSDVRFSSPQGQGESRTLTDSAGPRQITLRQFENGVTELQLSRPPLTSLVLSGGGAKGAAYPGAMLALEEKGMLDGIRSMSGSSAGGITAALLASGMSPAAFKTLSDKMDLISLLDSSNKKLKLFQHISSEIGASLKKGLGNKIGGFSELLLNVLPRIDSRAEPLERLLRDETRKAVLGQIATHPEVARQPTVAAIASRLQSGSGVTFGDLDRLSAYIPQIKTLNITGTAMFEGRPQLVVFNASHTPDLEVAQAAHISGSFPGVFQKVSLSDQPYQAGVEWTEFQDGGVMINVPVPEMIDKNFDSGPLRRNDNLILEFEGEAGEVAPDRGTRGGALKGWVVGVPALQAREMLQLEGLEELREQTVVVPLKSERGDFSGMLGGTLNFTMPDEIKAHLQERLQERVGEHLEKRLQASERHTFASLDEALLALDDSMLTSVAQQNPEITDGAVAFRQKARDAFTELTVAIVSANGLAGRLKLDEAMRSALQRLDALADTPERLAWLAAELNHADNVDHQQLLDAMRGQTVQSPVLAAALAEAQRRKVAVIAENIRKEVIFPSLYRPGQPDSNVALLRRAEEQLRHATSPAEINQALNDIVDNYSARGFLRFGKPLSSTTVEMAKAWRNKEFT

SEQ ID NO: 5 - protein, 17 aa, Artificial Sequence
  Synthetic polypeptide
MHIQSLQQSPSFAVELH

SEQ ID NO: 6 - protein, 54 aa, Artificial Sequence
  Synthetic polypeptide
MHIQSLQQSPSFAVELHQAASGRLGQIEARQVATPSEAQQLAQRQDAPKGEGLL

SEQ ID NO: 7 - protein, 96 aa, Artificial Sequence
  Synthetic polypeptide
MHIQSLQQSPSFAVELHQAASGRLGQIEARQVATPSEAQQLAQRQDAPKGEGLLARLGAALVRPFVAIMDWLGKLLGSHARTGPQPSQDAQPAVMS

SEQ ID NO: 8 - protein, 234 aa, Artificial Sequence
  Synthetic polypeptide
MHIQSLQQSPSFAVELHQAASGRLGQIEARQVATPSEAQQLAQRQDAPKGEGLLARLGAALVRPFVAIMDWLGKLLGSHARTGPQPSQDAQPAVMSSAVVFKQMVLQQALPMTLKGLDKASELATLTPEGLAREHSRLASGDGALRSLSTALAGIRAGSQVEESRIQAGRLLERSIGGIALQQWGTTGGAASQLVLDASPELRREITDQLHQVMSEVALLRQAVESEVSRVSAD

SEQ ID NO: 9 - DNA, 17 bp, Artificial Sequence
  Synthetic nucleic acid
ttcaccggggtggtgcc

SEQ ID NO: 10 - DNA, 17 bp, Artificial Sequence
  Synthetic nucleic acid
ctggacggcgacgtaaa

SEQ ID NO: 11 - DNA, 21 bp, Artificial Sequence
  Synthetic nucleic acid
gtaggactgaacgtcttgctc

SEQ ID NO: 12 - DNA, 21 bp, Artificial Sequence
  Synthetic nucleic acid
gatgggaggccatcacattgt

SEQ ID NO: 13 - DNA, 26 bp, Artificial Sequence
  Synthetic nucleic acid
cctacagctcctgggcaacgtgctgg

SEQ ID NO: 14 - DNA, 26 bp, Artificial Sequence
  Synthetic nucleic acid
ctggacgtagccttcgggcatggcgg

SEQ ID NO: 15 - DNA, 26 bp, Artificial Sequence
  Synthetic nucleic acid
ttttgagacaaggtcttgctctattg

SEQ ID NO: 16 - DNA, 26 bp, Artificial Sequence
  Synthetic nucleic acid
cagtattggctttgatgtaaagtact

SEQ ID NO: 17 - DNA, 72 bp, Artificial Sequence
  Synthetic nucleic acid
aggagctgttcaccggggtggtgcccatcctggtctagctggacggcgacgtaaacggccacaagttcagcg

SEQ ID NO: 18 - DNA, 72 bp, Artificial Sequence
  Synthetic nucleic acid
aggagctgttcaccggggtggtgcccatcctggtcgagctcgacggcgacgtaaacggccacaagttcagcg

SEQ ID NO: 19 - DNA, 72 bp, Artificial Sequence
  Synthetic nucleic acid
cctgattttatttctgtaggactgaacgtcttgcttgagatgtgatgaaggagatgggaggccatcacattg

SEQ ID NO: 20 - protein, 442 aa, Artificial Sequence
  Synthetic Polypeptide
MYQSLAMAANHGPPPGAYEAGGPGAFMHGAGAASSPVYVPTPRVPSSVLGLSYLQGGGAGSASGGASGGSSGGAASGAGPGTQQGSPGWSQAGADGAAYTPPPVSPRFSFPGTTGSLAAAAAAAAAREAAAYSSGGGAAGAGLAGREQYGRAGFAGSYSSPYPAYMADVGASWAAAAAASAGPFDSPVLHSLPGRANPAARHPNLDMFDDFSEGRECVNCGAMSTPLWRRDGTGHYLCNACGLYHKMNGINRPLIKPQRRLSASRRVGLSCANCQTTTTTLWRRNAEGEPVCNACGLYMKLHGVPRPLAMRKEGIQTRKRKPKNLNKSKTPAAPSGSESLPPASGASSNSSNATTSSSEEMRPIKTEPGLSSHYGHSSSVSQTFSVSAMSGHGPSIHPVLSALKLSPQGYASPVSQSPQTSSKQDSWNSLVLADSHGDIITA

SEQ ID NO: 21 - protein, 463 aa, Artificial Sequence
  Synthetic Polypeptide
MGRKKIQITRIMDERNRQVTFTKRKFGLMKKAYELSVLCDCEIALIIFNSTNKLFQYASTDMDKVLLKYTEYNEPHESRTNSDIVEALNKKENKGCESPDPDSSYALTPRTEEKYKKINEEFDNMIKSHKIPAVPPPNFEMPVSIPVSSHNSLVYSNPVSSLGNPNLLPLAHPSLQRNSMSPGVTHRPPSAGNTGGLMGGDLTSGAGTSAGNGYGNPRNSPGLLVSPGNLNKNMQAKSPPPMNLGMNNRKPDLRVLIPPGSKNTMPSVNQRINNSQSAQSLATPVVSVATPTLPGQGMGGYPSAISTTYGTEYSLSSADLSSLSGFNTASALHLGSVTGWQQQHLHNMPPSALSQLGACTSTHLSQSSNLSLPSTQSLNIKSEPVSPPRDRTTTPSRYPQHTRHEAGRSPVDSLSSCSSSYDGSDREDHRNEFHSPIGLTRPSPDERESPSVKRMRLSEGWAT

SEQ ID NO: 22 - protein, 518 aa, Artificial Sequence
  Synthetic Polypeptide
MADADEGFGLAHTPLEPDAKDLPCDSKPESALGAPSKSPSSPQAAFTQQGMEGIKVFLHERELWLKFHEVGTEMIITKAGRRMFPSYKVKVTGLNPKTKYILLMDIVPADDHRYKFADNKWSVTGKAEPAMPGRLYVHPDSPATGAHWMRQLVSFQKLKLTNNHLDPFGHIILNSMHKYQPRLHIVKADENNGFGSKNTAFCTHVFPETAFIAVTSYQNHKITQLKIENNPFAKGFRGSDDMELHRMSRMQSKEYPVVPRSTVRQKVASNHSPFSSESRALSTSSNLGSQYQCENGVSGPSQDLLPPPNPYPLPQEHSQIYHCTKRKEEECSTTDHPYKKPYMETSPSEEDSFYRSSYPQQQGLGASYRTESAQRQACMYASSAPPSEPVPSLEDISCNTWPSMPSYSSCTVTTVQPMDRLPYQHFSAHFTSGPLVPRLAGMANHGSPQLGEGMFQHQTSVAHQPVVRQCGPQTGLQSPGTLQPPEFLYSHGVPRTLSPHQYHSVHGVGMVPEWSDNS

SEQ ID NO: 23 - protein, 506 aa, Artificial Sequence
  Synthetic Polypeptide
MHIQSLQQSPSFAVELHQAASGRLGQIEARQVATPSEAQQLAQRQDAPKGEGLLDYKDDDDKELMYQSLAMAANHGPPPGAYEAGGPGAFMHGAGAASSPVYVPTPRVPSSVLGLSYLQGGGAGSASGGASGGSSGGAASGAGPGTQQGSPGWSQAGADGAAYTPPPVSPRFSFPGTTGSLAAAAAAAAAREAAAYSSGGGAAGAGLAGREQYGRAGFAGSYSSPYPAYMADVGASWAAAAAASAGPFDSPVLHSLPGRANPAARHPNLDMFDDFSEGRECVNCGAMSTPLWRRDGTGHYLCNACGLYHKMNGINRPLIKPQRRLSASRRVGLSCANCQTTTTTLWRRNAEGEPVCNACGLYMKLHGVPRPLAMRKEGIQTRKRKPKNLNKSKTPAAPSGSESLPPASGASSNSSNATTSSSEEMRPIKTEPGLSSHYGHSSSVSQTFSVSAMSGHGPSIHPVLSALKLSPQGYASPVSQSPQTSSKQDSWNSLVLADSHGDIITA

SEQ ID NO: 24 - protein, 527 aa, Artificial Sequence
  Synthetic Polypeptide
MHIQSLQQSPSFAVELHQAASGRLGQIEARQVATPSEAQQLAQRQDAPKGEGLLDYKDDDDKELMGRKKIQITRIMDERNRQVTFTKRKFGLMKKAYELSVLCDCEIALIIFNSTNKLFQYASTDMDKVLLKYTEYNEPHESRTNSDIVEALNKKENKGCESPDPDSSYALTPRTEEKYKKINEEFDNMIKSHKIPAVPPPNFEMPVSIPVSSHNSLVYSNPVSSLGNPNLLPLAHPSLQRNSMSPGVTHRPPSAGNTGGLMGGDLTSGAGTSAGNGYGNPRNSPGLLVSPGNLNKNMQAKSPPPMNLGMNNRKPDLRVLIPPGSKNTMPSVNQRINNSQSAQSLATPVVSVATPTLPGQGMGGYPSAISTTYGTEYSLSSADLSSLSGFNTASALHLGSVTGWQQQHLHNMPPSALSQLGACTSTHLSQSSNLSLPSTQSLNIKSEPVSPPRDRTTTPSRYPQHTRHEAGRSPVDSLSSCSSSYDGSDREDHRNEFHSPIGLTRPSPDERESPSVKRMRLSEGWAT

SEQ ID NO: 25 - protein, 582 aa, Artificial Sequence
  Synthetic Polypeptide
MHIQSLQQSPSFAVELHQAASGRLGQIEARQVATPSEAQQLAQRQDAPKGEGLLDYKDDDDKELMADADEGFGLAHTPLEPDAKDLPCDSKPESALGAPSKSPSSPQAAFTQQGMEGIKVFLHERELWLKFHEVGTEMIITKAGRRMFPSYKVKVTGLNPKTKYILLMDIVPADDHRYKFADNKWSVTGKAEPAMPGRLYVHPDSPATGAHWMRQLVSFQKLKLTNNHLDPFGHIILNSMHKYQPRLHIVKADENNGFGSKNTAFCTHVFPETAFIAVTSYQNHKITQLKIENNPFAKGFRGSDDMELHRMSRMQSKEYPVVPRSTVRQKVASNHSPFSSESRALSTSSNLGSQYQCENGVSGPSQDLLPPPNPYPLPQEHSQIYHCTKRKEEECSTTDHPYKKPYMETSPSEEDSFYRSSYPQQQGLGASYRTESAQRQACMYASSAPPSEPVPSLEDISCNTWPSMPSYSSCTVTTVQPMDRLPYQHFSAHFTSGPLVPRLAGMANHGSPQLGEGMFQHQTSVAHQPVVRQCGPQTGLQSPGTLQPPEFLYSHGVPRTLSPHQYHSVHGVGMVPEWSDNS

SEQ ID NO: 26 - protein, 1380 aa, Artificial Sequence
  Synthetic Polypeptide
MASSPPKKKRKVSWKDASGWSRMHADPIRPRRPSPARELLPGPQPDRVQPTADRGVSAPAGSPLDGLPARRTVSRTRLPSPPAPSPAFSAGSFSDLLRPFDPSLLDTSLLDSMPAVGTPHTAAAPAEWDEAQSALRAADDPPPTVRVAVTAARPPRAKPAPRRRAAQPSDASPAAQVDLRTLGYSQQQQEKIKPKVRSTVAQHHEALVGHGFTHAHIVALSQHPAALGTVAVTYQHIITALPEATHEDIVGVGKQWSGARALEALLTDAGELRGPPLQLDTGQLVKIAKRGGVTAMEAVHASRNALTGAPLETGAATGRVPFAIQAAQLLGRAIGAGLFAITPAGERGMCCKAIKLGNARVFPVTTLLTPDQVVAIASNGGGKQALETVQRLLPVLCQDHGLTPDQVVAIASNGGGKQALETVQRLLPVLCQDHGLTPDQVVAIASHDGGKQALETVQRLLPVLCQDHGLTPDQVVAIASNIGGKQALETVQRLLPVLCQDHGLTPDQVVAIASHDGGKQALETVQRLLPVLCQDHGLTPDQVVAIASHDGGKQALETVQRLLPVLCQDHGLTPDQVVAIASNNGGKQALETVQRLLPVLCQDHGLTPDQVVAIASNNGGKQALETVQRLLPVLCQDHGLTPDQVVAIASNNGGKQALETVQRLLPVLCQDHGLTPDQVVAIASNNGGKQALETVQRLLPVLCQDHGLTPDQVVAIASNGGGKQALETVQRLLPVLCQDHGLTPDQVVAIASNNGGKQALETVQRLLPVLCQDHGLTPDQVVAIASNNGGKQALETVQRLLPVLCQDHGLTPDQVVAIASNGGGKQALETVQRLLPVLCQDHGLTPDQVVAIASNNGGKQALETVQRLLPVLCQDHGLTPDQVVAIASHDGGKQALETVQRLLPVLCQDHGLTPDQVVAIASHDGGKQALETVQRLLPVLCQDHGMSELTHINCVALTARFPPVVSNDHLVALACLGGRPAMDAVKKGLPHAPELIRRVNRRIGERTSHRVADYAQVVRVLEFFQCHSHPAYAFDEAMTQFGMSRNGLVQLFRRVGVTELEARGGTLPPASQRWDRILQASGMKRAKPSPTSAQTPDQASLHAFADSLERDLDAPSPMHEGDQTRASSRKRSRSDRAVTGPSAQQAVEVRVPEQRDALHLPLSWRVKRPRTRIWGGLPDPISRSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINF

SEQ ID NO: 27 - protein, 1376 aa, Artificial Sequence
  Synthetic Polypeptide
MNNKPYSIGLDIGTNSVGWAVITDDYKVPSKKMKVLGNTDKHFIKKNLLGALLFDEGTTAEDRRLKRTARRRYTRRKNRLRYLQEIFTEEMSKVDISFFHRLDDSFLVPEDKRGSKYPIFATLEEEKEYHKNFPTIYHLRKHLADSKEKADFRLIYLALAHIIKYRGHFLYEESFDIKNNDIQKIFNEFISIYDNTFEGSSLNGQNAQVEAIFTDKISKSAKRERVLKLFPDEKSTGLFSEFLKLIVGNQADFKKHFDLEEKAPLQFSKDTYDEDLENLLVQIGDDFADLFLVAKKLYDAILLSGILTVTDPSTKAPLSASMIDRYENHQKDLAALKQFIKTNLPEKYDEVFSDQSKDGYAGYIDGKTTQEAFYKYIKNLLSKLEGADYFLDKIEREDFLRKQRTFDNGSIPHQIHLQEMNAIIRRQGEHYPFLQENKEKIEKILTFRIPYYVGPLARGNRDFAWLTRNSDQAIRPWNFEEIVDKARSAEDFINKMTNYDLYLPEEKVLPKHSLLYETFAVYNELTKVKFIAEGLRDYQFLDSGQKQQIVTQLFKEKRKVTEKDIIQYLHNVDSYDGIELKGIEKQFNASLSTYHDLLKIIKDKEFMDDSKNEAILENIVHTLTIFEDREMIKQRLAHYASIFDEKVIKALTRRHYTGWGKLSAKLINGIYDKQSKKTILDYLIDDGEINRNFMQLINDDGLSFKEIIQKAQVVGKTNDVKQVVQELPGSPAIKKGILQSIKLVDELVKVMGHAPESIVIEMARENQTTARGKKNSQQRYKRIEDALKNLAHGLDSNILKEHPTDNIQLQNDRLFLYYLQNGKDMYTGKSLDINQLSSCDIDHIIPQAFIKDDSLDNRVLTSSKDNRGKSDNVPSLEIVQKRKAFWQQLLDSKLISERKFNNLTKAERGGLDERDKVGFIKRQLVETRQITKHVAQILDARFNTEVTEKDKKDRSVKIITLKSNLVSNFRKEFRLYKVREINDYHHAHDPYLNAVVAKAILKKYPKLEPEFVYGDYQKYDLKRYISRTKDPKEVEKATEKYFFYSNLLNFFKEEVHYADGTIVKRENIEYSKDTGEIAWNKEKDFATIKKVLSLPQVNIVKKTEEQTVGQNGGLFDNNIVSKKKVVDASKLTPIKSGLSPEKYGGYARPTIAYSVLVIADIEKGKAKKLKRIKEMVGITVQDKKKFEANPIAYLEECGYKNINPNLIIKLPKYSLFEFNNGQRRLLASSIELQKGNELIVPYHFTALLYHAQRINKISEPIHKQYVETHQSEFKELLTAIISLSKKYIQKPNVESLLQQAFDQSDKDIYQLSESFISLLKLISFGAPGTFKFLGVEISQSNVRYQSVSSCFNATLIHQSITGLYETRIDLSKLGED

SEQ ID NO: 28 - protein, 1444 aa, Artificial Sequence
  Synthetic Polypeptide
MHIQSLQQSPSFAVELHQAASGRLGQIEARQVATPSEAQQLAQRQDAPKGEGLLDYKDDDDKELMASSPPKKKRKVSWKDASGWSRMHADPIRPRRPSPARELLPGPQPDRVQPTADRGVSAPAGSPLDGLPARRTVSRTRLPSPPAPSPAFSAGSFSDLLRPFDPSLLDTSLLDSMPAVGTPHTAAAPAEWDEAQSALRAADDPPPTVRVAVTAARPPRAKPAPRRRAAQPSDASPAAQVDLRTLGYSQQQQEKIKPKVRSTVAQHHEALVGHGFTHAHIVALSQHPAALGTVAVTYQHIITALPEATHEDIVGVGKQWSGARALEALLTDAGELRGPPLQLDTGQLVKIAKRGGVTAMEAVHASRNALTGAPLETGAATGRVPFAIQAAQLLGRAIGAGLFAITPAGERGMCCKAIKLGNARVFPVTTLLTPDQVVAIASNGGGKQALETVQRLLPVLCQDHGLTPDQVVAIASNGGGKQALETVQRLLPVLCQDHGLTPDQVVAIASHDGGKQALETVQRLLPVLCQDHGLTPDQVVAIASNIGGKQALETVQRLLPVLCQDHGLTPDQVVAIASHDGGKQALETVQRLLPVLCQDHGLTPDQVVAIASHDGGKQALETVQRLLPVLCQDHGLTPDQVVAIASNNGGKQALETVQRLLPVLCQDHGLTPDQVVAIASNNGGKQALETVQRLLPVLCQDHGLTPDQVVAIASNNGGKQALETVQRLLPVLCQDHGLTPDQVVAIASNNGGKQALETVQRLLPVLCQDHGLTPDQVVAIASNGGGKQALETVQRLLPVLCQDHGLTPDQVVAIASNNGGKQALETVQRLLPVLCQDHGLTPDQVVAIASNNGGKQALETVQRLLPVLCQDHGLTPDQVVAIASNGGGKQALETVQRLLPVLCQDHGLTPDQVVAIASNNGGKQALETVQRLLPVLCQDHGLTPDQVVAIASHDGGKQALETVQRLLPVLCQDHGLTPDQVVAIASHDGGKQALETVQRLLPVLCQDHGMSELTHINCVALTARFPPVVSNDHLVALACLGGRPAMDAVKKGLPHAPELIRRVNRRIGERTSHRVADYAQVVRVLEFFQCHSHPAYAFDEAMTQFGMSRNGLVQLFRRVGVTELEARGGTLPPASQRWDRILQASGMKRAKPSPTSAQTPDQASLHAFADSLERDLDAPSPMHEGDQTRASSRKRSRSDRAVTGPSAQQAVEVRVPEQRDALHLPLSWRVKRPRTRIWGGLPDPISRSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINF

SEQ ID NO: 29 - protein, 1440 aa, Artificial Sequence
  Synthetic Polypeptide
MHIQSLQQSPSFAVELHQAASGRLGQIEARQVATPSEAQQLAQRQDAPKGEGLLDYKDDDDKELMNNKPYSIGLDIGTNSVGWAVITDDYKVPSKKMKVLGNTDKHFIKKNLLGALLFDEGTTAEDRRLKRTARRRYTRRKNRLRYLQEIFTEEMSKVDISFFHRLDDSFLVPEDKRGSKYPIFATLEEEKEYHKNFPTIYHLRKHLADSKEKADFRLIYLALAHIIKYRGHFLYEESFDIKNNDIQKIFNEFISIYDNTFEGSSLNGQNAQVEAIFTDKISKSAKRERVLKLFPDEKSTGLFSEFLKLIVGNQADFKKHFDLEEKAPLQFSKDTYDEDLENLLVQIGDDFADLFLVAKKLYDAILLSGILTVTDPSTKAPLSASMIDRYENHQKDLAALKQFIKTNLPEKYDEVFSDQSKDGYAGYIDGKTTQEAFYKYIKNLLSKLEGADYFLDKIEREDFLRKQRTFDNGSIPHQIHLQEMNAIIRRQGEHYPFLQENKEKIEKILTFRIPYYVGPLARGNRDFAWLTRNSDQAIRPWNFEEIVDKARSAEDFINKMTNYDLYLPEEKVLPKHSLLYETFAVYNELTKVKFIAEGLRDYQFLDSGQKQQIVTQLFKEKRKVTEKDIIQYLHNVDSYDGIELKGIEKQFNASLSTYHDLLKIIKDKEFMDDSKNEAILENIVHTLTIFEDREMIKQRLAHYASIFDEKVIKALTRRHYTGWGKLSAKLINGIYDKQSKKTILDYLIDDGEINRNFMQLINDDGLSFKEIIQKAQVVGKTNDVKQVVQELPGSPAIKKGILQSIKLVDELVKVMGHAPESIVIEMARENQTTARGKKNSQQRYKRIEDALKNLAHGLDSNILKEHPTDNIQLQNDRLFLYYLQNGKDMYTGKSLDINQLSSCDIDHIIPQAFIKDDSLDNRVLTSSKDNRGKSDNVPSLEIVQKRKAFWQQLLDSKLISERKFNNLTKAERGGLDERDKVGFIKRQLVETRQITKHVAQILDARFNTEVTEKDKKDRSVKIITLKSNLVSNFRKEFRLYKVREINDYHHAHDPYLNAVVAKAILKKYPKLEPEFVYGDYQKYDLKRYISRTKDPKEVEKATEKYFFYSNLLNFFKEEVHYADGTIVKRENIEYSKDTGEIAWNKEKDFATIKKVLSLPQVNIVKKTEEQTVGQNGGLFDNNIVSKKKVVDASKLTPIKSGLSPEKYGGYARPTIAYSVLVIADIEKGKAKKLKRIKEMVGITVQDKKKFEANPIAYLEECGYKNINPNLIIKLPKYSLFEFNNGQRRLLASSIELQKGNELIVPYHFTALLYHAQRINKISEPIHKQYVETHQSEFKELLTAIISLSKKYIQKPNVESLLQQAFDQSDKDIYQLSESFISLLKLISFGAPGTFKFLGVEISQSNVRYQSVSSCFNATLIHQSITGLYETRIDLSKLGED

SEQ ID NO: 30 - DNA, 20 bp, Artificial Sequence
  Synthetic Polynucleotide
tctcactatgggcacagcag

SEQ ID NO: 31 - DNA, 20 bp, Artificial Sequence
  Synthetic Polynucleotide
gggacagcttcagagcagac

SEQ ID NO: 32 - DNA, 21 bp, Artificial Sequence
  Synthetic Polynucleotide
atcccgatgcagacgattcag

SEQ ID NO: 33 - DNA, 21 bp, Artificial Sequence
  Synthetic Polynucleotide
aacagcacacaatctttgcct

SEQ ID NO: 34 - DNA, 21 bp, Artificial Sequence
  Synthetic Polynucleotide
actggccttaatcccaaaacg

SEQ ID NO: 35 - DNA, 22 bp, Artificial Sequence
  Synthetic Polynucleotide
acggaccatttgttatcagcaa

SEQ ID NO: 36 - DNA, 20 bp, Artificial Sequence
  Synthetic Polynucleotide
tcccgagacccagttcatag

SEQ ID NO: 37 - DNA, 20 bp, Artificial Sequence
  Synthetic Polynucleotide
ttctttggcatcaaggaagg

SEQ ID NO: 38 - DNA, 20 bp, Artificial Sequence
  Synthetic Polynucleotide
gagaacccctacttccacgg

SEQ ID NO: 39 - DNA, 21 bp, Artificial Sequence
  Synthetic Polynucleotide
gacagggccatactgtagtcg

SEQ ID NO: 40 - DNA, 20 bp, Artificial Sequence
  Synthetic Polynucleotide
acattttacccgggagccta

SEQ ID NO: 41 - DNA, 19 bp, Artificial Sequence
  Synthetic Polynucleotide
ggctttgtccagctccact

SEQ ID NO: 42 - DNA, 20 bp, Artificial Sequence
  Synthetic Polynucleotide
ccagctaaaggctgagagga

SEQ ID NO: 43 - DNA, 19 bp, Artificial Sequence
  Synthetic Polynucleotide
aggcgtagtcgtatgggtt

SEQ ID NO: 44 - DNA, 20 bp, Artificial Sequence
  Synthetic Polynucleotide
ttgctgacaggatgcagaag

SEQ ID NO: 45 - DNA, 20 bp, Artificial Sequence
  Synthetic Polynucleotide
gtacttgcgctcaggaggag

SEQ ID NO: 46 - DNA, 53 bp, Artificial Sequence
  Synthetic Polynucleotide
ctgttcaccggggtggtgcccatcctggtcgagctggacggcgacgtaaacgg

SEQ ID NO: 47 - DNA, 53 bp, Artificial Sequence
  Synthetic Polynucleotide
ccgtttacgtcgccgtccagctcgaccaggatgggcaccaccccggtgaacag

SEQ ID NO: 48 - DNA, 60 bp, Artificial Sequence
  Synthetic Polynucleotide
ctgttcaccggggtggtgcccatcctggtcgagctggacggcgacgtaaacggccacaag

SEQ ID NO: 49 - DNA, 40 bp, Artificial Sequence
  Synthetic Polynucleotide
ctgttcaccggggtggtacggcgacgtaaacggccacaag

SEQ ID NO: 50 - DNA, 27 bp, Artificial Sequence
  Synthetic Polynucleotide
ctgttcaccggggtggtcggccacaag

SEQ ID NO: 51 - DNA, 29 bp, Artificial Sequence
  Synthetic Polynucleotide
ctgttcaccggggtggtcccggccacaag

SEQ ID NO: 52 - DNA, 61 bp, Artificial Sequence
  Synthetic Polynucleotide
ctgttcaccggggtggtgccccatcctggtcgagctggacggcgacgtaaacggccacaag

SEQ ID NO: 53 - DNA, 60 bp, Artificial Sequence
  Synthetic Polynucleotide
ctgttcaccggggtggtgcccatcctggtcgagctggacgacgacgtaaacggccacaag

SEQ ID NO: 54 - DNA, 83 bp, Artificial Sequence
  Synthetic Polynucleotide
atggtgagcaagggcgaggagctgttcaccggggtggtgcccatcctggtcgagctggacggcgacgtaaacggccacaagtt

SEQ ID NO: 55 - DNA, 84 bp, Artificial Sequence
  Synthetic Polynucleotide
atggtgagcaagggcgaggagctgttcaccggggtggtgcccatcctggtctagctggacggcgacgtaaacggccacaagttc

SEQ ID NO: 56 - DNA, 84 bp, Artificial Sequence
  Synthetic Polynucleotide
atggtgagcaagggcgaggagctgttcaccggggtggtgcccatcctggtcgagctggacggcgacgtaaacggccacaagttc

SEQ ID NO: 57 - DNA, 62 bp, Artificial Sequence
  Synthetic Polynucleotide
ctgtaggactgaacgtcttgctcgagatgtgatgaaggagatgggaggccatcacattgtag

SEQ ID NO: 58 - DNA, 62 bp, Artificial Sequence
  Synthetic Polynucleotide
ctacaatgtgatggcctcccatctccttcatcacatctcgagcaagacgttcagtcctacag

SEQ ID NO: 59 - DNA, 77 bp, Artificial Sequence
  Synthetic Polynucleotide
gattttatttctgtaggactgaacgtcttgctcgagatgtgatgaaggagatgggaggccatcacattgtagccctc

SEQ ID NO: 60 - DNA, 51 bp, Artificial Sequence
  Synthetic Polynucleotide
gattttatttctgtaggactgaacgtcttgctcgatcacattgtagccctc

SEQ ID NO: 61 - DNA, 38 bp, Artificial Sequence
  Synthetic Polynucleotide
gattttatttcttgggaggccatcacattgtagccctc

SEQ ID NO: 62 - DNA, 56 bp, Artificial Sequence
  Synthetic Polynucleotide
gattttatttctgtaggactgaacgtcttgcgggaggccatcacattgtagccctc

SEQ ID NO: 63 - DNA, 45 bp, Artificial Sequence
  Synthetic Polynucleotide
gattttatttctctgctggagggaggccatcacattgtagccctc

SEQ ID NO: 64 - DNA, 35 bp, Artificial Sequence
  Synthetic Polynucleotide
gattttatttctgtaggactgaacgtcttgctctc

SEQ ID NO: 65 - DNA, 88 bp, Artificial Sequence
  Synthetic Polynucleotide
attaaattcctgattttatttctgtaggactgaacgtcttgctcgagatgtgatgaaggagatgggaggccatcacattgtagccctc

SEQ ID NO: 66 - DNA, 88 bp, Artificial Sequence
  Synthetic Polynucleotide
attaaattcctgattttatttctgtaggactgaacgtcttgcttgagatgtgatgaaggagatgggaggccatcacattgtagccctc

SEQ ID NO: 67 - DNA, 77 bp, Artificial Sequence
  Synthetic Polynucleotide
gattttatttctgtaggactgaacgtcttgcttgagatgtgatgaaggagatgggaggccatcacattgtagccctc

SEQ ID NO: 68 - DNA, 50 bp, Artificial Sequence
  Synthetic Polynucleotide
gattaaattcctgattttatttctgtaggactgaacgtcttgctgccctc

SEQ ID NO: 69 - DNA, 79 bp, Artificial Sequence
  Synthetic Polynucleotide
gattaaattcctgattttatttctgtaggactgaacggatgtgatgaaggagatgggaggccatcacattgtagccctc

SEQ ID NO: 70 - DNA, 57 bp, Artificial Sequence
  Synthetic Polynucleotide
gattttatttctgtaggactgaacgaggagatgggaggccatcacattgtagccctc